(19) United States
(12) Patent Application Publication
(10) Pub. No.: US 2009/0163380 A1
(43) Pub. Date: Jun. 25, 2009

(54) ANALYTE FOCUSING BIOCHIPS FOR AFFINITY MASS SPECTROMETRY

(75) Inventors: Mark L. Stolowitz, Pleasanton, CA (US); Allan H. Stephan, Seattle, WA (US)

Correspondence Address: SEED INTELLECTUAL PROPERTY LAW GROUP PLLC, 701 FIFTH AVE, SUITE 5400, SEATTLE, WA, 98104, US

(73) Assignee: Stratos Biosystems, LLC, Seattle, WA (US)

(21) Appl. No.: 12/269,188

(22) Filed: Nov. 12, 2008

Related U.S. Application Data
(63) Continuation of application No. PCT/US2007/011470, filed on May 11, 2007.
(60) Provisional application No. 60/892,604, filed on Mar. 2, 2007.
(60) Provisional application No. 60/799,964, filed on May 12, 2006.

(51) Int. Cl. C40B 50/18; C40B 40/04; C07C 321/04

(52) U.S. Cl. 506/15; 506/32; 562/426

(57) ABSTRACT

A novel affinity capture surface, as well as methods of using and making the affinity capture surface and sample presentation devices or biochips comprising the affinity capture surface, are disclosed. The affinity capture surface comprising a substrate surface having adjacent first and second affinity capture zones, wherein the first affinity capture zone comprises a first binary self-assembled monolayer comprising a plurality of affinity capture monomers and hydrophilic-terminated monomers associated with the substrate surface and the second affinity capture zone comprises a second binary self-assembled monolayer comprising a plurality of affinity capture monomers and hydrophobic-terminated monomers associated with the substrate surface, and wherein the affinity capture monomers are capable of selectively retaining an analyte and are cleavable to release terminal portions of the affinity capture monomers and the analyte, thereby generating a hydrophilic surface in the first affinity capture zone and a hydrophobic surface in the second affinity capture zone.

### CROSS-REFERENCE TO RELATED APPLICATIONS

This application is a continuation of International PCT Patent Application No. PCT/US2007/011470, filed May 11, 2007, now pending, which claims the benefit of U.S. Provisional Patent Application No. 60/799,964, filed May 12, 2006, and U.S. Provisional Patent Application No. 60/892,604, filed Mar. 2, 2007. These applications are incorporated herein by reference in their entireties.

### BACKGROUND OF THE INVENTION

1. Field of the Invention

The present invention relates to biochips that enable the initial fractionation and subsequent focusing of target analytes for affinity mass spectrometry by matrix-assisted laser desorption/ionization mass spectrometry, and to novel alkanethiols and self-assembled monolayers comprised thereof.

2. Description of Related Art

Binary self-assembled monolayers (SAMs) of alkanethiols on gold, comprised of both specificity-conferring and protein adsorption resistant monomers, are now routinely utilized to prepare surfaces for a variety of bioanalytical devices including: atomic force microscopy; biosensor; matrix-assisted laser desorption/ionization time-of-flight mass spectrometry; surface plasmon resonance; and quartz crystal microbalance. Collectively, these techniques offer advantages over traditional colorimetric and fluorescence techniques in that they enable label-free detection.

To a great extent, the success attributed to the use of binary SAMs in the above and other applications has resulted from the development and recent commercial availability of protein adsorption resistant monomers that effectively minimize nonspecific adsorption of proteins. The first monolayers shown to exhibit protein adsorption resistant properties were those having pendant oligo(ethylene oxide) moieties (see, e.g, Prime, K. L. and Whitesides, G. M. Science 1991, 12, 1164-1167; and Pale-Grosdemagne, C; Simon, E. S.; Prime, K. L. and Whitesides, G. M. J. Amer. Chem. Soc. 1991, 113, 12-20). Many of the structural factors which confer protein adsorption resistance upon oligo(ethylene oxide) terminated monomers have been elucidated (see, e.g., Herrwerth, S.; Eck, W.; Reinhardt, S. and Grunze, M. 2003, 125, 9359-9366; Feldman, K.; Hahner, G.; Spencer, N. D.; Harder, P. and Grunze, M. J. Amer. Chem. Soc. 1999, 121, 10134-10141; Sigal, G. B.; Mrksich, M. and Whitesides, G. M. J. Amer. Chem. Soc. 1998, 120, 3464-3473; and Wamg, R. L. C.; Kreuzer, H. J. and Grunze, M. J. Phys. Chem. B 1997, 101, 9767-9773). Systematic studies have been undertaken to identify structural motifs other than oligo(ethylene oxide) that confer protein adsorption resistance and alternative motifs have been proposed (see, e.g., Luk, Y.-Y.; Kato, M. and Mrksich, M. Langmuir 2000, 16, 9604-9608; Chapman, R. G.; Ostuni, E.; Takayama, S.; Holmin, R. E.; Yan, L. and Whitesides, G. M. J. Amer. Chem. Soc. 2000, 122, 8303-8304; Holmin, R. E.; Chen, X.; Chapman, R. G.; Takayama, S, and Whitesides, G. M. Langmuir 2001, 17, 2841-2850; Ostuni, E.; Chapman, R. G.; Holmlin, R. E.; Takayama, S, and Whitesides, G. M. Langmuir 2001, 17, 5605-5620; and Ostuni, E.; Chapman, R. G.; Liang, M. N.; Meluleni, G.; Pier, G.; Ingber, D. E. and Whitesides, G. M. Langmuir 2001, 17, 6336-6343). Nevertheless, oligo(ethylene oxide) terminated monomers remain the reagents of choice when preparing protein adsorption resistant self-assembled monolayers.

In recent years, considerable interest has emerged in the development of dynamic SAMs with properties that are altered in response to external stimuli including applied potential, pH and temperature. Many of the dynamic monolayers reported thus far exploit electrochemical oxidation or reduction of immobilized moieties, and in particular immobilized hydroquinone, to effect an alteration of surface properties. Applications reported thus far have included: (1) electrochemical deprotection for site-selective immobilization (see, e.g., Yeo, W. S, and Mrksich, M. Advanced Materials 2004, 16(15), 1352-1356; and Kim, K.; Yang, H.; Kim, E.; Han, Y. B.; Kim, Y. T.; Kang, S. H. and Kwak, J. Langmuir 2002, 18, 1460-1462); (2) electrochemically induced covalent coupling (see, e.g., Kim, K.; Jang, M.; Yang, H.; Kim, E.; Kim, Y. T. and Kwak, J. Langmuir 2004, 20, 3821-3823; Houseman, B. T. and Mrksich, M. TRENDS in Biotechnology 2002, 20(7), 279-281; Yousaf, M. N.; Houseman, B. T. and Mrksich, M. PNAS 2001, 98, 5992-5996; and Yousaf, M. and Mrksich, M. J. Amer. Chem. Soc. 1999, 121, 4286-4287); and (3) protein patterning based on electrochemical activation (see, e.g., Kim, K.; Yang, H.; Jon, S.; Kim, E. and Kwak, J. J. Amer. Chem. Soc. 2004, 126, 15368-15369; and Dillmore, W. S.; Yousaf, M. N. and Mrksich, M. Langmuir 2004, 20, 7223-7231).

Of particular interest is the development of dynamic SAMs that alter the display of ligands, and hence, the interactions of cells and proteins with surfaces (see, e.g., Hodneland, C. D. and Mrksich, M. Langmuir 1997, 13, 6001-6003; Hodneland, C. D. and Mrksich, M. J. Amer. Chem. Soc. 2000, 122, 4235-4236; Yeo, W. S.; Hodneland, C. D. and Mrksich, M. ChemBioChem 2001, 7(8), 590-593); and Yeo, W. S.; Yousaf, M. N. and Mrksich, M. J. Amer. Chem. Soc. 2003, 125, 14994-14995).

Dynamic SAMs that selectively release immobilized ligands in response to an applied potential are disclosed in U.S. Pat. No. 6,764,768, issued Jul. 20, 2004. In this instance, electrochemically active monomers having pendant ligands that confer specificity are exploited in conjunction with oligo(ethylene oxide) terminated monomers that minimize nonspecific adsorption of protein.

Collectively, dynamic SAMs that release immobilized ligands in response to applied potentials suffer from a significant shortcoming that results from the residual presence of the electrochemically active moiety. Despite the utilization of protein absorption resistant oligo(ethylene oxide) terminated background monomers, the aromatic ring structures associated with the electrochemically active moieties which have been exploited thus far exhibit hydrophobic properties that give rise to the nonspecific adsorption of protein. This limitation was acknowledged in U.S. Pat. No. 6,764,768, as evidenced by the following passage (column 23, lines 37-44): “It is preferable that the monolayers remain inert to the non-specific adsorption of protein, both before and after release of the ligand. Accordingly, the monomers used here present the moiety of the present invention at low density (approximately 1% of total alkanethiolate) surrounded by tri(ethylene glycol) groups because the latter are highly effective at preventing non-specific adsorption of protein.” Although limiting the density of the electrochemically active moiety may reduce the problem of nonspecific adsorption of protein, it also results in a significant reduction in binding capacity with a concomitant reduction in the overall utility of the surface.

Recently, binary self-assembled monolayers of alkanethiols on gold, comprised of both specificity-conferring and protein adsorption resistant monomers, have been exploited in conjunction with matrix-assisted laser/desorption ionization time-of-flight mass spectrometry (MALDI-TOF-MS) to enable label-free detection of enzymatic activity, protein-ligand interactions and protein-protein interactions. This methodology, termed SAMDI (self-assembled monolayers for MALDI) mass spectrometry, has been described in a series of publications (see, e.g. Min, D.-H., Su, J. and Mrksich, M. Angew. Chem. Int. Ed. 2004, 43, 5973-5977; Min, D.-H., Tang, W.-J. and Mrksich, M. Nature Biotech. 2004, 22, 717-723; Min, D.-H., Yeo, W.-S, and Mrksich, M. Anal. Chem. 2004, 76(14), 3923-3929; and Yeo, W.-S., Min, D.-H., Hsieh, R. W., Greene, G. L. and Mrksich, M. Angew. Chem. Int. Ed. 2005, 44, 5480-5483).

Mass spectrometry (MS) employing matrix-assisted laser/desorption ionization has become increasingly important in life sciences research, as recognized by the 2002 Nobel Prize in chemistry. Mass spectrometry has emerged as the method of choice for the analysis of complex protein samples, and MS-based proteomics is now routinely exploited for the discovery and validation of biomarkers. A mass spectrometer consists of an ion source, mass analyzer that measures the mass-to-charge (m/z) ratio of ionized analytes, and a detector that counts the number of ions at each m/z value. MALDI and electrospray ionization (ESI) are the two techniques most often utilized to ionize peptides and proteins for mass spectrometric analysis. MALDI ionizes analytes residing in a crystalline matrix via laser desorption, whereas ESI ionizes dissolved analytes in solution and is most often coupled to a liquid separation system (such as high performance liquid chromatograph). MALDI is exploited in conjunction with time-of-flight (TOF) mass analyzers and is utilized to measure the mass of intact peptides and small proteins. To enable the fragmentation of MALDI-generated ions for the purpose of peptide-mass fingerprinting, MALDI ion sources have recently been coupled to quadrupole ion-trap, TOF-TOF and quadrupole-TOF mass spectrometers.

Historically, polished stainless steel sample supports were utilized to introduce samples into MALDI mass spectrometers. Recently, stainless steel sample supports have been modified with hydrophobic thin films to afford surfaces that limit the spreading of aqueous sample droplets. More recently, the development and commercial availability of sample supports having functionalized surfaces that enable either the fractionation or concentration of analytes have significantly increased the utility of MS-based proteomics involving MALDI-MS. Most recently, sample supports having functionalized surfaces that enable both the initial fractionation and subsequent concentration of analytes have been introduced. Collectively, functionalized sample supports reduce or eliminate losses often encountered when small volumes of biological fluids are manipulated for the purpose of fractionation or concentration prior to spotting on the surface of a MALDI sample support.

Functionalized sample supports that enable the fractionation of analytes were first described in U.S. Pat. No. 5,719,060, issued Feb. 17, 1998. This patent was the first in a series that collectively describe an approach referred to as surface-enhanced laser desorption/Ionization (SELDI), which exploits either immobilized chemical moieties or immobilized proteins including antibodies to selectively retain target analytes from a complex sample on the sample support surface. SELDI biochips are offered commercially as the ProteinChip® from Bio-Rad (Hercules, Calif.), and have been utilized primarily for protein profiling and immunoaffinity mass spectrometry. However, the SELDI approach has recently been the subject of intense criticism owing to performance issues that result from the limited sensitivity and mass resolution afforded by this general approach.

Functionalized sample supports that enable the concentration of analytes are described in U.S. Pat. Nos. 6,287,872 and 6,952,011, issued Sep. 11, 2001 and Oct. 4, 2005, respectively. These patents describe the use of either hydrophilic anchors or laser-etched regions, respectively, in conjunction with hydrophobic surface treatments to localize small analyte-containing aqueous droplets on the surface of the sample support just prior to the crystallization of the matrix (due to the attraction of the hydrophilic anchor or laser etched region for the aqueous droplet). As compared to samples applied directly to the surface of a hydrophobic sample support, this approach affords an increase in sensitivity of detection of greater than 10-fold and a concomitant reduction in sample heterogeneity. Sample supports that exploit hydrophilic anchors are offered commercially as the Anchor Chip® from Bruker Daltronik (Bremen, GmbH), and are utilized exclusively in conjunction with MALDI mass spectrometers manufactured by Bruker Daltronik. Sample supports that exploit laser etched zones were briefly offered commercially as MassPREP Targets™ from Waters (Milford, Mass.), but were removed from the market when it was demonstrated that hydrophobic peptides could not be efficiently recovered from the extremely hydrophobic surface of the sample support.

Recently, functionalized sample supports that claim to enable the initial fractionation and subsequent concentration of analytes were described in U.S. Patent Application Publications Nos. US2002/0045270 and US2004/0197921. These patent applications describe hydrophilic anchors utilized in conjunction with “areas of affinity adsorbents adjacent to the hydrophilic anchors for purifying biosubstances”. The aforementioned patent applications were submitted by the inventors of the Anchor Chip®, and are assigned to Bruker Daltronik. However, sample supports of this type have not been offered commercially and to date preliminary data discussing their utility has not been presented at scientific forums.

Very recently, additional functionalized sample supports that enable the initial fractionation and subsequent concentration of analytes were described in U.S. Patent Application Publication No. US 2005/0164402. This patent application describes the use of patterned self-assembled monolayers on the surface of the sample support to effect the manipulation of analytes. Procedurally, analytes are initially retained in an affinity adsorbent zone and then allowed to dry on the surface of the sample support. Matrix solution is next applied and dissolves the deposited analyte. Finally, as the matrix solution evaporates, a small analyte-containing aqueous droplet is localized on the surface of the sample support above a hydrophilic zone just prior to the crystallization of matrix. Sample supports of this type have very recently been offered commercially as Mass Spec Focus Chips™ from Qiagen (Hilden, GmbH), and are available in a variety of formats to enable their use in mass spectrometers manufactured by several companies. However, at present only three moderately hydrophobic surface chemistries (reverse phase, moderately hydrophobic and metal ion interaction) are available.

The U.S. patent application publications referenced hereinabove (namely, U.S. Patent Application Publications Nos. 2002/0045270, 2004/0197921 and 2005/0164402) claim utility with respect to the use of a variety of affinity interactions. However, in practice only those interactions involving surfaces that are moderately hydrophobic or hydrophobic are useful in conjunction with the disclosed inventions. This significant limitation results from the requirement that the surface energies in the affinity adsorbent and hydrophilic zones be sufficiently different so as to ensure the localization of an analyte-containing droplet over the hydrophilic zone prior to the crystallization of matrix. In particular, the use of biological affinity interactions involving immobilized antibodies or other proteins is precluded, as surfaces having immobilized proteins are moderately hydrophilic or hydrophilic and do not differ significantly with respect to surface energy from the hydrophilic zones required for localization of the analyte-containing droplet.

Accordingly, although there have been advances in the field, there remains a need for dynamic SAMs that selectively release immobilized ligands in response to applied potentials, are devoid of nonspecific adsorption resulting from the residual presence of an electrochemically active moiety, and exhibit no limitations with respect to binding capacity. In addition, there remains a need for functionalized sample supports for MALDI mass spectrometry that enable the initial fractionation and subsequent concentration of analytes, and are compatible with the use of biological affinity interactions involving immobilized antibodies or other proteins. The present invention addresses these needs and provides further related advantages.

### SUMMARY OF THE INVENTION

In brief, the present invention relates to novel biochips that enable the practice of affinity mass spectrometry by matrix-assisted laser desorption/ionization mass spectrometry. In addition, the present invention provides biochips, methods of using biochips, and methods of fabricating biochips. In addition, the present invention relates to novel alkanethiols and self-assembled monolayers comprised thereof, which release pendant affinity capture moieties in response to either an applied oxidizing potential or contact with an oxidizing solution to yield self-assembled monolayers comprised of protein adsorption resistant monomers.

In a first embodiment, the present invention provides a method for preparing an analyte-containing solution, the method comprising the steps of: (a) providing an affinity capture surface comprising a substrate surface having adjacent first and second affinity capture zones, wherein: (i) the first affinity capture zone comprises a first binary self-assembled monolayer comprising a plurality of affinity capture monomers and hydrophilic-terminated monomers associated with the substrate surface; and (ii) the second affinity capture zone comprises a second binary self-assembled monolayer comprising a plurality of affinity capture monomers and hydrophobic-terminated monomers associated with the substrate surface, wherein the affinity capture monomers are capable of selectively retaining an analyte; (b) contacting the affinity capture surface with the analyte to form analyte/affinity capture monomer complexes between the analyte and the affinity capture monomers; and (c) cleaving the affinity capture monomers to release terminal portions of the affinity capture monomers and the analyte into a solution in contact with the affinity capture surface, thereby yielding the analyte-containing solution, generating a hydrophilic surface in the first affinity capture zone and generating a hydrophobic surface in the second affinity capture zone.

In further embodiments, the analyte-containing solution is an analyte-containing liquid droplet and the method further comprises the step of evaporating the analyte-containing liquid droplet to transfer the evaporated analyte-containing liquid droplet to the hydrophilic first affinity capture zone.

In more specific embodiments, the first affinity capture zone is surrounded by the second affinity capture zone and, optionally, the substrate surface of the affinity capture surface further has a non-wettable common surrounding zone surrounding the first and second affinity capture zones. The non-wettable common surrounding zone may comprise a non-wettable self-assembled monolayer comprising a plurality of non-wettable monomers.

In various embodiments, the first binary self-assembled monolayer is comprised of at most 20% of the affinity capture monomers and at least 80% of the hydrophilic-terminated monomers, and the second binary self-assembled monolayer is comprised of at most 20% of the affinity capture monomers and at least 80% of the hydrophobic-terminated monomers. In other embodiments, the first binary self-assembled monolayer is comprised of at most 10% of the affinity capture monomers and at least 90% of the hydrophilic-terminated monomers, and the second binary self-assembled monolayer is comprised of at most 10% of the affinity capture monomers and at least 90% of the hydrophobic-terminated monomers. In other embodiments, the first binary self-assembled monolayer is comprised of 5% of the affinity capture monomers and 95% of the hydrophilic-terminated monomers, and the second binary self-assembled monolayer is comprised of 5% of the affinity capture monomers and at least 95% of the hydrophobic-terminated monomers.

In more specific embodiments, the affinity capture monomers have one of the following general formulas:

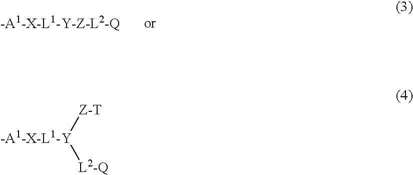

wherein:
A¹ is an anchoring moiety associated with the substrate surface;
X is alkylene;
L¹ is absent or is a protein adsorption resistant moiety;
Y is —O—Y¹, wherein Y¹ is aryl or substituted aryl, and wherein Y is bonded to L¹ and Z such that L¹ and Z are oriented in either an ortho or para relationship;
Z is a leaving group;
L² is absent or is a protein adsorption resistant moiety;
Q is an affinity capture moiety or reactive moiety; and
T is hydrogen, alkylene or aryl.

More specifically, the affinity capture monomers may have the general formula:

-A¹-X-L¹-Y-Z-L²-Q (3)

wherein:
A¹ is —S— or —SCH₂CH₂CONH—;
X is C₃-C₁₈ alkylene;
L¹ is —(OCH₂CH₂)ₘ—, wherein m is an integer from 3 to 6;
Y is —O—Y¹, wherein Y¹ is aryl or substituted aryl, and wherein Y is bonded to L¹ and Z such that L¹ and Z are oriented in either an ortho or para relationship;
Z is —OCO—, —OCOO—, —OCONH—, —OSO₂—, —OPO₃—, —OCH₂COO—, —OCH₂CONH—, —OCH₂CH₂—, —OCH₂C₆H₄—, —OC₆H₄—, —OSi(CH₃)₂—, —OSi(CH₂CH₃)₂—, —OSi[CH(CH₃)₂]₂— or —OSi(C₆H₅)₂—;
L² is —(CH₂)ₙ— or —(CH₂CH₂O)ₙₙ—, wherein n an integer from 2 to 8 and nn is an integer from 2 to 6; and
Q is an affinity capture moiety or reactive moiety.

For example, in a more specific embodiment:
A¹ is —S—;
X is —(CH₂)₁₁—;
L¹ is —(OCH₂CH₂)ₘ—, wherein m is 3 or 4;
Y is —O(C₆H₄)— and is bonded to L¹ and Z such that L¹ and Z are oriented in a para relationship;
Z is —OCH₂CONH—, —OCONH— or —OSi[CH(CH₃)₂]₂—;
L² is —(CH₂CH₂O)ₙₙ—, wherein nn is an integer from 2 to 6; and
Q is an affinity capture moiety or reactive moiety.

In other more specific embodiments, the hydrophilic-terminated monomers have the general formula:

-A¹-X-L¹-T¹

wherein T¹ is a hydrophilic terminator, and the hydrophobic-terminated monomers have the general formula:

-A¹-X-L¹-T²

wherein T² is a hydrophobic terminator.

For example, in a more specific embodiment:
A¹ is —S— or —SCH₂CH₂CONH—;
X is C₃-C₁₈ alkylene;
L¹ is —(OCH₂CH₂)ₘ—, wherein m is an integer from 3 to 6;
T¹ is —OH, —OCH₂CONH₂, —OCONH₂ and —OCOCH₂OH;
T² is —OCH₃, —OCH₂CH₃, —OCOCH₃ and —OCOCF₃;
Y is —O—Y¹, wherein Y¹ is aryl or substituted aryl, and wherein Y is bonded to L¹ and Z such that L¹ and Z are oriented in either an ortho or para relationship;
Z is —OCO—, —OCOO—, —OCONH—, —OSO₂—, —OPO₃—, —OCH₂COO—, —OCH₂CONH—, —OCH₂CH₂—, —OCH₂C₆H₄—, —OC₆H₄—, —OSi(CH₃)₂—, —OSi(CH₂CH₃)₂—, —OSi[CH(CH₃)₂]₂— or —OSi(C₆H₅)₂—;
L² is —(CH₂)ₙ— or —(CH₂CH₂O)ₙₙ—, wherein n an integer from 2 to 8 and nn is an integer from 2 to 6; and
Q is an affinity capture moiety or reactive moiety.

In various embodiments, the affinity capture monomers are cleaved by electrochemical, chemical or photochemical means.

In other various embodiments, the substrate surface comprises glass, metal, a polymeric material or silica. For example, the substrate surface may comprise gold or silver.

In a second embodiment, the present invention provides an affinity capture surface comprising a substrate surface having adjacent first and second affinity capture zones, wherein: (a) the first affinity capture zone comprises a first binary self-assembled monolayer comprising a plurality of affinity capture monomers and hydrophilic-terminated monomers associated with the substrate surface; and (b) the second affinity capture zone comprises a second binary self-assembled monolayer comprising a plurality of affinity capture monomers and hydrophobic-terminated monomers associated with the substrate surface, and wherein the affinity capture monomers are capable of selectively retaining an analyte and are cleavable to release terminal portions of the affinity capture monomers and the analyte, thereby generating a hydrophilic surface in the first affinity capture zone and a hydrophobic surface in the second affinity capture zone.

In more specific embodiments, the first affinity capture zone is surrounded by the second affinity capture zone and, optionally, the substrate surface of the affinity capture surface further has a non-wettable common surrounding zone surrounding the first and second affinity capture zones. The non-wettable common surrounding zone may comprise a non-wettable self-assembled monolayer comprising a plurality of non-wettable monomers.

In various embodiments, the first binary self-assembled monolayer is comprised of at most 20% of the affinity capture monomers and at least 80% of the hydrophilic-terminated monomers, and the second binary self-assembled monolayer is comprised of at most 20% of the affinity capture monomers and at least 80% of the hydrophobic-terminated monomers. In other embodiments, the first binary self-assembled monolayer is comprised of at most 10% of the affinity capture monomers and at least 90% of the hydrophilic-terminated monomers, and the second binary self-assembled monolayer is comprised of at most 10% of the affinity capture monomers and at least 90% of the hydrophobic-terminated monomers. In other embodiments, the first binary self-assembled monolayer is comprised of 5% of the affinity capture monomers and 95% of the hydrophilic-terminated monomers, and the second binary self-assembled monolayer is comprised of 5% of the affinity capture monomers and at least 95% of the hydrophobic-terminated monomers.

In more specific embodiments, the affinity capture monomers have one of the following general formulas:

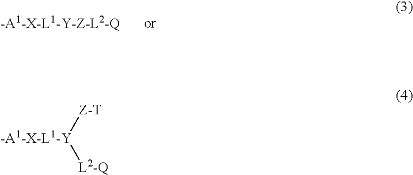

wherein:
A¹ is an anchoring moiety associated with the substrate surface;
X is alkylene;
L¹ is absent or is a protein adsorption resistant moiety;
Y is —O—Y¹, wherein Y¹ is aryl or substituted aryl, and wherein Y is bonded to L¹ and Z such that L¹ and Z are oriented in either an ortho or para relationship;
Z is a leaving group;
L² is absent or is a protein adsorption resistant moiety;
Q is an affinity capture moiety or reactive moiety; and
T is hydrogen, alkylene or aryl.

More specifically, the affinity capture monomers may have the general formula:

-A¹-X-L¹-Y-Z-L²-Q (3)

wherein:
A¹ is —S— or —SCH₂CH₂CONH—;
X is C₃-C₁₈ alkylene;
L¹ is —(OCH₂CH₂)ₘ—, wherein m is an integer from 3 to 6;
Y is —O—Y¹, wherein Y¹ is aryl or substituted aryl, and wherein Y is bonded to L¹ and Z such that L¹ and Z are oriented in either an ortho or para relationship;
Z is —OCO—, —OCOO—, —OCONH—, —OSO₂—, —OPO₃—, —OCH₂COO—, —OCH₂CONH—, —OCH₂CH₂—, —OCH₂C₆H₄—, —OC₆H₄—, —OSi(CH₃)₂—, —OSi(CH₂CH₃)₂—, —OSi[CH(CH₃)₂]₂— or —OSi(C₆H₅)₂—;
L² is —(CH₂)ₙ— or —(CH₂CH₂O)ₙₙ—, wherein n an integer from 2 to 8 and nn is an integer from 2 to 6; and
Q is an affinity capture moiety or reactive moiety.

For example, in a more specific embodiment:
A¹ is —S—,
X is —(CH₂)₁₁—;
L¹ is —(OCH₂CH₂)ₘ—, wherein m is 3 or 4;
Y is —O(C₆H₄)— and is bonded to L¹ and Z such that L¹ and Z are oriented in a para relationship;
Z is —OCH₂CONH—, —OCONH— or —OSi[CH(CH₃)₂]₂—;
L² is —(CH₂CH₂O)ₙₙ—, wherein nn is an integer from 2 to 6; and
Q is an affinity capture moiety or reactive moiety.

In other more specific embodiments, the hydrophilic-terminated monomers have the general formula:

-A¹-X-L¹-T¹

wherein T¹ is a hydrophilic terminator, and the hydrophobic-terminated monomers have the general formula:

-A¹-X-L¹-T²

wherein T² is a hydrophobic terminator.

For example, in a more specific embodiment:
A¹ is —S— or —SCH₂CH₂CONH—;
X is C₃-C₁₈ alkylene;
L¹ is —(OCH₂CH₂)ₘ—, wherein m is an integer from 3 to 6;
T¹ is —OH, —OCH₂CONH₂, —OCONH₂ and —OCOCH₂OH;
T² is —OCH₃, —OCH₂CH₃, —OCOCH₃ and —OCOCF₃;
Y is —O—Y¹, wherein Y¹ is aryl or substituted aryl, and wherein Y is bonded to L¹ and Z such that L¹ and Z are oriented in either an ortho or para relationship;
Z is —OCO—, —OCOO—, —OCONH—, —OSO₂—, —OPO₃—, —OCH₂COO—, —OCH₂CONH—, —OCH₂CH₂—, —OCH₂C₆H₄—, —OC₆H₄—, —OSi(CH₃)₂—, —OSi(CH₂CH₃)₂—, —OSi[CH(CH₃)₂]₂— or —OSi(C₆H₅)₂—;
L² is —(CH₂)ₙ— or —(CH₂CH₂O)ₙₙ—, wherein n an integer from 2 to 8 and nn is an integer from 2 to 6; and
Q is an affinity capture moiety or reactive moiety.

In various embodiments, the substrate surface comprises glass, metal, a polymeric material or silica. For example, the substrate surface may comprise gold or silver.

In a third embodiment, the present invention provides a sample presentation device or biochip comprising the foregoing affinity capture surface.

In a fourth embodiment, the present invention provides a method of making the foregoing affinity capture surface comprising the steps of: (a) providing the substrate surface; (b) associating the second binary self-assembled monolayer with the substrate surface; (c) ablating the second binary self-assembled monolayer from the substrate surface in the first affinity capture zone; and (d) associating the first binary self-assembled monolayer with the ablated surface in the first affinity capture zone.

In a fifth embodiment, the present invention provides a compound having one of the following general formulas:

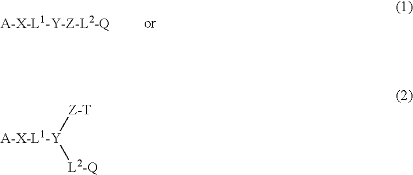

wherein:
A is an anchoring moiety;
X is alkylene;
L¹ is absent or is a protein adsorption resistant moiety;
Y is —O—Y¹, wherein Y¹ is aryl or substituted aryl, and wherein Y is bonded to L¹ and Z such that L¹ and Z are oriented in either an ortho or para relationship;
Z is a leaving group;
L² is absent or is a protein adsorption resistant moiety;
Q is an affinity capture moiety or reactive moiety; and
T is hydrogen, alkylene or aryl.

More specifically, the compound may have the general formula:

A-X-L¹-Y-Z-L²-Q (1)

wherein:
A is HS— or HSCH₂CH₂CONH—;
X is C₃-C₁₈ alkylene;
L¹ is —(OCH₂CH₂)ₘ—, wherein m is an integer from 3 to 6;
Y is —O—Y¹, wherein Y¹ is aryl or substituted aryl, and wherein Y is bonded to L¹ and Z such that L¹ and Z are oriented in either an ortho or para relationship;
Z is —OCO—, —OCOO—, —OCONH—, —OSO₂—, —OPO₃—, —OCH₂COO—, —OCH₂CONH—, —OCH₂CH₂—, —OCH₂C₆H₄—, —OC₆H₄—, —OSi(CH₃)₂—, —OSi(CH₂CH₃)₂—, —OSi[CH(CH₃)₂]₂— or —OSi(C₆H₅)₂—;
L² is —(CH₂)ₙ— or —(CH₂CH₂O)ₙₙ—, wherein n an integer from 2 to 8 and nn is an integer from 2 to 6; and
Q is an affinity capture moiety or reactive moiety.

For example, in a more specific embodiment:
A is HS—;
X is —(CH₂)₁₁—;
L¹ is —(OCH₂CH₂)ₘ—, wherein m is 3 or 4;
Y is —O(C₆H₄)— and is bonded to L¹ and Z such that L¹ and Z are oriented in a para relationship;
Z is —OCH₂CONH—, —OCONH— or —OSi[CH(CH₃)₂]₂—;
L² is —(CH₂CH₂O)ₙₙ—, wherein nn is an integer from 2 to 6; and
Q is an affinity capture moiety or reactive moiety.

In a specific embodiment, the compound may be:

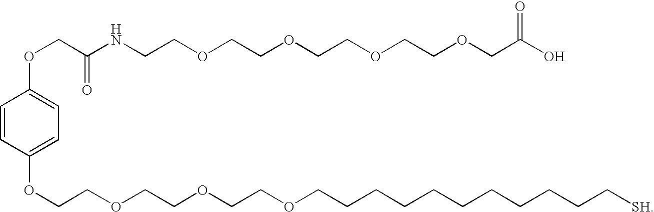

In a sixth embodiment, the present invention provides a self-assembled monolayer comprising: (a) a substrate surface; and (b) a plurality of affinity capture monomers immobilized on the substrate surface, wherein the affinity capture monomers have one of the following general formulas:

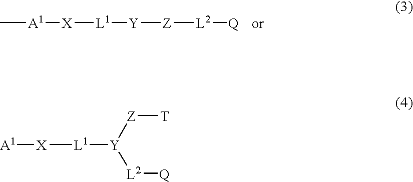

wherein:
A¹ is an anchoring moiety;
X is alkylene;
L¹ is absent or is a protein adsorption resistant moiety;
Y is —O—Y¹, wherein Y¹ is aryl or substituted aryl, and wherein Y is bonded to L¹ and Z such that L¹ and Z are oriented in either an ortho or para relationship;
Z is a leaving group;
L² is absent or is a protein adsorption resistant moiety;
Q is an affinity capture moiety or reactive moiety; and
T is hydrogen, alkylene or aryl.

More specifically, the affinity capture monomers may have the general formula:

-A¹-X-L¹-Y-Z-L²-Q (3)

wherein:
A¹ is —S— or —SCH₂CH₂CONH—;
X is C₃-C₁₈ alkylene;
L¹ is —(OCH₂CH₂)ₘ—, wherein m is an integer from 3 to 6;
Y is —O—Y¹, wherein Y¹ is aryl or substituted aryl, and wherein Y is bonded to L¹ and Z such that L¹ and Z are oriented in either an ortho or para relationship;
Z is —OCO—, —OCOO—, —OCONH—, —OSO₂—, —OPO₃—, —OCH₂COO—, —OCH₂CONH—, —OCH₂CH₂—, —OCH₂C₆H₄—, —OC₆H₄—, —OSi(CH₃)₂—, —OSi(CH₂CH₃)₂—, —OSi[CH(CH₃)₂]₂— or —OSi(C₆H₅)₂—;
L² is —(CH₂)ₙ— or —(CH₂CH₂O)ₙₙ—, wherein n an integer from 2 to 8 and nn is an integer from 2 to 6; and
Q is an affinity capture moiety or reactive moiety.

For example, in a more specific embodiment:
A¹ is —S—;
X is —(CH₂)₁₁—;
L¹ is —(OCH₂CH₂)ₘ—, wherein m is 3 or 4;
Y is —O(C₆H₄)— and is bonded to L¹ and Z such that L¹ and Z are oriented in a para relationship;
Z is —OCH₂CONH—, —OCONH— or —OSi[CH(CH₃)₂]₂—;
L² is —(CH₂CH₂O)ₙₙ—, wherein nn is an integer from 2 to 6; and
Q is an affinity capture moiety or reactive moiety.

In further embodiments, the self-assembled monolayer further comprises: (a) a plurality of hydrophilic-terminated monomers, immobilized on the substrate surface, having the structure:

-A¹-X-L¹-T¹

wherein T¹ is a hydrophilic terminator; or (b) a plurality of hydrophobic-terminated monomers, immobilized on the substrate surface, having the structure:

-A¹-X-L¹-T²

wherein T² is a hydrophobic terminator.

For example, in a more specific embodiment:
A¹ is —S— or —SCH₂CH₂CONH—;
X is C₃-C₁₈ alkylene;
L¹ is —(OCH₂CH₂)ₘ—, wherein m is an integer from 3 to 6;
T¹ is —OH, —OCH₂CONH₂, —OCONH₂ and —OCOCH₂OH;
T² is —OCH₃, —OCH₂CH₃, —OCOCH₃ and —OCOCF₃;
Y is —O—Y¹, wherein Y¹ is aryl or substituted aryl, and wherein Y is bonded to L¹ and Z such that L¹ and Z are oriented in either an ortho or para relationship;
Z is —OCO—, —OCOO—, —OCONH—, —OSO₂—, —OPO₃—, —OCH₂COO—, —OCH₂CONH—, —OCH₂CH₂—, —OCH₂C₆H₄—, —OC₆H₄—, —OSi(CH₃)₂—, —OSi(CH₂CH₃)₂—, —OSi[CH(CH₃)₂]₂— or —OSi(C₆H₅)₂—;
L² is —(CH₂)ₙ— or —(CH₂CH₂O)ₙₙ—, wherein n an integer from 2 to 8 and nn is an integer from 2 to 6; and
Q is an affinity capture moiety or reactive moiety.

In various embodiments, the substrate surface comprises glass, metal, a polymeric material or silica. For example, the substrate surface may comprise gold or silver.

These and other aspects of the invention will be apparent upon reference to the attached figures and following detailed description.

### BRIEF DESCRIPTION OF THE FIGURES

FIG. 1a depicts a representative embodiment of a biochip of the present invention, wherein the central first affinity capture zone and the second affinity capture zone are concentric with respect to one another, and wherein the second affinity capture zone is surrounded by the non-wettable common surrounding zone.

FIG. 1b depicts a cross-sectional view of the biochip depicted in FIG. 1a.

FIG. 2 depicts the surface of a representative biochip of the present invention, wherein the surface is further comprised of 24 pairs of first and second affinity capture zones, wherein the first and second affinity capture zones are concentric with respect to one another, and wherein pairs of affinity capture zones are surrounded by a non-wettable common surrounding zone.

FIG. 3 depicts the surfaces of four representative biochips of the present invention, wherein the biochips are arranged to form a regularly-spaced 8 by 12 element array of concentric first and second affinity capture zones, and wherein the elements of said array correspond to the spacing associated with the industry standard multiwell plate.

FIG. 4 depicts a cross-sectional view corresponding to one pair of affinity capture zones and the common surrounding zone of the affinity capture surface of the present invention, wherein each of the individual monomers is depicted, and wherein the binary self-assembled monolayers associated with the first and second affinity capture zones are depicted, and wherein the self-assembled monolayer associated with the non-wettable common surrounding zone is depicted.

FIG. 5 depicts the localization of a liquid droplet on the surface of the affinity capture surface of the present invention that results from the presence of the non-wettable monomers in the non-wettable common surrounding zone.

FIGS. 6a through 6c depict a series of manipulations involving: sample application, incubation and washing, which collectively enable the selective retention of a target analyte on the surface of the affinity capture surface.

FIGS. 7a and 7b depict the electrochemical cleavage of the affinity capture monomers in the first and second affinity capture zones resulting in release of the retained analyte in conjunction with the affinity capture moiety or reactive moiety (e.g., ligand or interactant) as well as the oxidized form of the cleavable moiety (i.e., the terminal portion of the affinity capture monomer).

FIG. 8 depicts a representative embodiment of the binary self-assembled monolayers associated with the first and second affinity capture zones, as well as the electrochemical cleavage of an affinity capture monomer having a pendant affinity capture moiety or reactive moiety (e.g., ligand or interactant).

FIGS. 9a through 9c depict the progressive evaporation of an analyte-containing liquid droplet with eventual localization or transfer or the evaporated analyte-containing liquid droplet to the hydrophilic first affinity capture zone.

FIGS. 10a and 10b depict the process of laser desorption which occurs in the inlet of the MALDI mass spectrometer.

FIGS. 11a through 11e depict the self-assembled monolayer deposition and UV-photopatterning steps involved in the fabrication of biochips of the present invention.

FIGS. 12a and 12b depict a self-assembled monolayer comprised of hydrophilic-terminated monomers suitable for use in conjunction with the first affinity capture zone of the biochip of the present invention, as well as an image of a water droplet applied to the surface of the depicted self-assembled monolayer.

FIGS. 13a and 13b depict a self-assembled monolayer comprised of hydrophobic-terminated monomers suitable for use in conjunction with the second affinity capture zone of the biochip of the present invention, as well as an image of a water droplet applied to the surface of the depicted self-assembled monolayer.

FIGS. 14a and 14b depict a self-assembled monolayer comprised of non-wettable monomers suitable for use in conjunction with the non-wettable common surrounding zone of the biochip of the present invention, as well as an image of a water droplet applied to the surface of the depicted self-assembled monolayer.

FIG. 15 is a representative mass spectrum of human Transthyretin obtained as set forth in Example 3.

FIGS. 16a and 16b show the deposition of sinapinic acid matrix crystals on representative patterned biochips as set forth in Example 5.

FIGS. 17a, 17b and 17c are representative mass spectra corresponding to human serum sample volumes of 10.0, 5.0 and 2.5 μL, respectively, as set forth in Example 5.

FIG. 18 (Synthetic Scheme 1) outlines the step-wise synthesis of an alkanethiol of formula (IV), a precursor to the alkanethiols of the present invention.

FIG. 19 (Synthetic Scheme 2) outlines the step-wise synthesis of two synthetic intermediates utilized to prepare alkanethiols of the present invention.

FIG. 20 (Synthetic Scheme 3) outlines the step-wise synthesis of a representative alkanethiol of formula (XII).

FIGS. 21a and 21b depict the electrochemical cleavage of a representative self-assembled monolayer and the corresponding cyclic voltammogram.

FIG. 22 (Synthetic Scheme 4) outlines the step-wise synthesis of a representative alkanethiol of formula (XVIII).

FIGS. 23a and 23b depict the electrochemical cleavage of a representative binary self-assembled monolayer and the corresponding cyclic voltammogram.

FIG. 24 (Synthetic Scheme 5) outlines the step-wise synthesis of a representative alkanethiol of formula (XXI).

FIG. 25 (Synthetic Scheme 6) outlines the step-wise synthesis of a representative alkanethiol of formula (XXVI).

FIG. 26 (Synthetic Scheme 7) outlines the step-wise synthesis of a representative alkanethiol of formula (XXXI).

FIG. 27 (Synthetic Scheme 8) outlines the step-wise synthesis of a representative alkanethiol of formula (XXXIV).

### DETAILED DESCRIPTION OF THE INVENTION

As noted above, the present invention relates to novel biochips that enable the practice of affinity mass spectrometry by matrix-assisted laser desorption/ionization mass spectrometry. In addition, the present invention provides biochips, methods of using biochips, and methods of fabricating biochips. In addition, the present invention relates to novel alkanethiols and self-assembled monolayers comprised thereof, which release pendant affinity capture moieties in response to either an applied oxidizing potential or contact with an oxidizing solution to yield self-assembled monolayers comprised of protein adsorption resistant monomers.

The following detailed description should be read with reference to the figures, in which identical reference numerals refer to like elements through out the different figures. The drawings are not necessarily to scale, depict selected embodiments and are not intended to limit the scope of the invention.

### DEFINITIONS

Unless defined otherwise, all technical and scientific terms used herein have the meaning commonly understood by a person skilled in the art to which this invention belongs. As used herein, the following terms have the meanings ascribed to them unless specified otherwise.

“Adsorption” refers to the process by which an analyte is retained on a surface as a consequence of interactions, such as chemical bonding (covalent or non-covalent), between the analyte and the surface.

“Analyte” and “target analyte” refer to a component of a multi-component sample that is desirably detected.

“Substrate” refers to a preferably flat material capable of supporting a surface.

“Surface” refers to the exterior or upper boundary of a body or a substrate.

“Wettability” refers to the degree to which a solid surface is wetted by an aqueous liquid droplet.

“Matrix” refers to materials used in mass spectrometry techniques, such as MADDI-MS and SELDI-MS, for adsorbing the energy of a laser and transferring that energy to analyte molecules, thereby enabling ionization of labile macromolecules. Compounds frequently used as matrices for the detection of biological analytes include, but are not limited to, 3,5-dimethoxy-4-hydroxycinnanamic acid (sinapinic acid, SA), α-cyano-4-hydroxycinnamic acid (HCCA) and 2,5-dihydroxybenzoic acid (DHBA). Many other suitable matrices are known to those skilled in the art.

“Self-assembled monolayer” refers molecular assemblies (Unimolecular thin films) that are formed spontaneously by the immersion of an appropriate substrate into a solution of active monomers in an appropriate solvent.

“Alkanethiol” means a compound containing an alkyl group bonded to a thiol (—SH) group.

“Alkylene” refers to a substituted or unsubstituted, straight, branched or cyclic hydrocarbon chain, preferably containing from 1 to 20 carbon atoms (C₁-C₂₀). Representative lower alkylene groups typically contain from 1 to 6 carbon atoms (C₁-C₆). Representative cycloalkylenes typically have from 3 to 10, preferably 3 to 6, carbon atoms in their ring structure. Suitable examples of unsubstituted alkylene groups include methylene (—CH₂—), 2-propylene (—CH₂—CH(CH₃H), and cyclohexylene (—C₆H₁₀—) wherein the carbon atoms form a six-membered ring, and the like.

“Aryl” refers to any aromatic carbocyclic or heteroaromatic group, preferably of 3 to 10 carbon atoms. The aryl group can be monocyclic (i.e. phenyl) or polycyclic (i.e., naphthyl) and can be unsubstituted or substituted. Preferred aryl groups include phenyl, naphthyl, furyl, thienyl, pyridyl, indolyl, quinolinyl or isoquinolinyl.

“Substituted” means that the moiety contains at least one, preferably 1 to 3 substituent(s). Suitable substituents include hydrogen (—H), hydroxyl (—OH), amino (—NH₂), oxy (—O—), carbonyl (—CO—), thiol, alkyl, alkenyl, alkynyl, alkoxy, halo, nitrile, nitro, aryl, and heterocyclic groups. These substituents can optionally be further substituted with 1 to 3 substituents. Examples of substituted substituents include carboxamide, alkylamino, dialkylamino, carboxylate, alkoxycarbonyl, alkylaryl, arylalkyl, alkylheterocyclic, and the like.

### Overview

The present invention provides novel biochips having an affinity capture surface comprising one or more pairs of concentric affinity capture zones (first and second affinity capture zones) and a common surrounding zone, wherein the first affinity capture zone is comprised of a first binary self-assembled monolayer comprising a plurality of affinity capture monomers and hydrophilic-terminated monomers, wherein the second affinity capture zone is comprised of a second binary self-assembled monolayer comprising a plurality of affinity capture monomers and hydrophobic-terminated monomers, wherein the non-wettable common surrounding zone is comprised of a non-wettable self-assembled monolayer comprising a plurality of non-wettable monomers, wherein the first affinity capture zone is surrounded by the second affinity capture zone, and wherein the second affinity capture zone is surrounded by the non-wettable common surrounding zone.

The affinity capture monomers common to both first and second affinity capture zones enable the selective retention of a target analyte from an aqueous sample and include a electrochemically, chemically or photochemically cleavable linker. The hydrophilic-terminated and hydrophobic-terminated monomers associated with the first and second affinity capture zones, respectively, render the surface of the biochip wettable and resistant to non-specific adsorption of protein. Neither aqueous nor organic liquid droplets wet the non-wettable monomers associated with the common surrounding zone.

Cleavage of the affinity capture monomers affords two concentric zones that differ significantly with respect to surface energy, wherein said first zone (namely, the first affinity capture zone) is hydrophilic, wherein said second zone (namely, the second affinity capture zone) is moderately hydrophobic, and wherein the difference in surface energy between the first and second zones is sufficient to enable the localization of an evaporating analyte-containing liquid droplet on the surface of the biochip in the area corresponding to the first affinity capture zone.

Protein adsorption resistant self-assembled monolayers having traceless cleavable linkers suitable for use in conjunction with the biochips of the present invention are described in detail herein and were described in detail in U.S. Patent Application No. 60/799,963, filed May 12, 2006, and U.S. Patent Application No. 60/892,604, filed Mar. 2, 2007, which applications are assigned to the assignee of the present application and are incorporated herein by reference in its entirety.

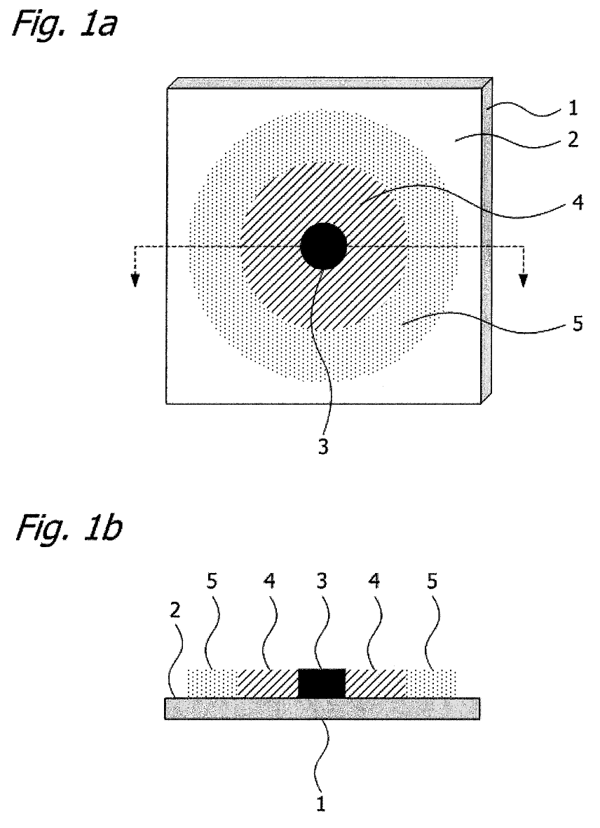

With reference to FIGS. 1a and 1b, a representative biochip of the present invention is depicted, being comprised of a substrate 1 having a conducting metal surface 2, wherein the surface is further comprised of contiguous zones being concentric with respect to one another, wherein the central zone 3 is the first affinity capture zone, wherein the second affinity capture zone 4 surrounds the first affinity capture zone, and wherein the common surrounding zone 5 surrounds the second affinity capture zone. In more specific embodiments, the first affinity capture zone 3 has a diameter of from about 0.4 to 0.8 mm, and the second affinity capture zone 4 has a diameter of from about 2.0 to 4.0 mm. In yet further more specific embodiments, the first affinity capture zone 3 has a diameter of 0.6 mm, and the second affinity capture zone 4 has a diameter of 3.0 mm.

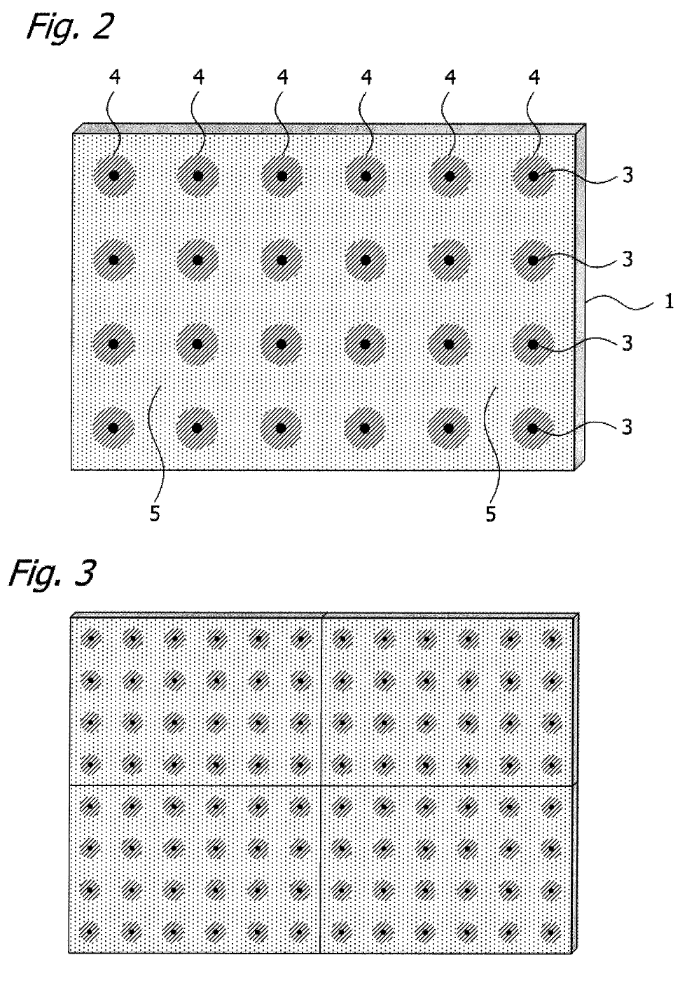

With reference to FIG. 2, a representative biochip of the present invention is shown comprising a substrate 1 having a conducting metal surface 2, wherein the surface is further comprised of 24 concentric pairs of first affinity capture zones 3 and second affinity capture zones 4, all of which are surrounded by a common surrounding zone 5. In certain embodiments, concentric pairs of affinity capture zones are arrayed on one of either 2.25 mm, 4.5 mm and 9.0 mm centers, which correspond to the spacing associated with industry standard multiwell plates.

With reference to FIG. 3, biochips of the present invention may be grouped such that concentric pairs of first and second affinity capture zones collectively provide one of either 96, 384 and 1536 concentric pairs of affinity capture zones, which correspond to industry standard multiwell plates, thereby enabling robotic sample processing on industry standard liquid-handling robots.

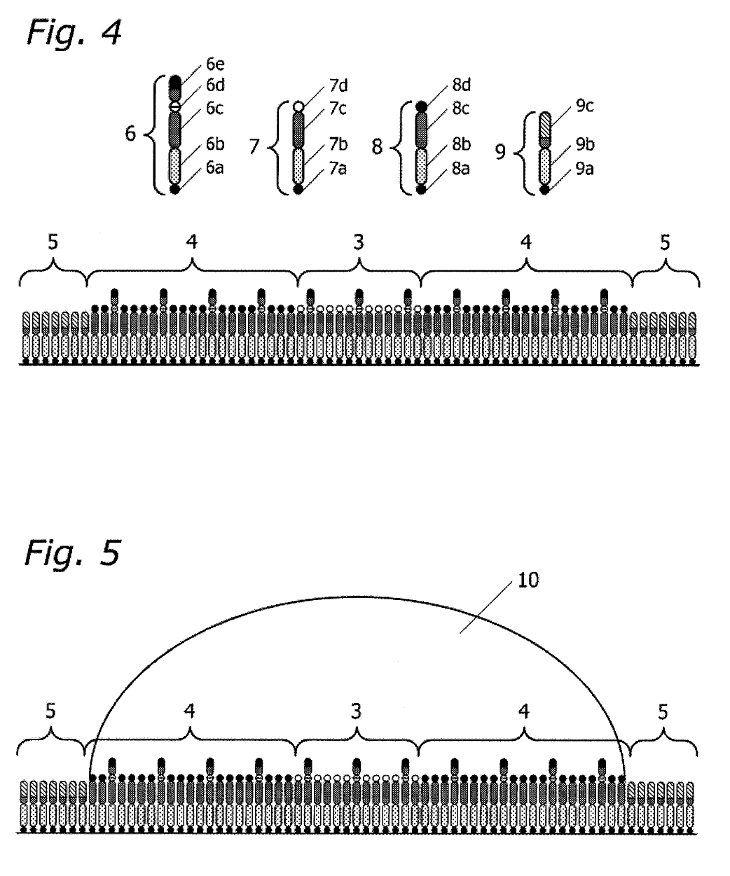

With reference to FIG. 4, a cross-sectional view of the affinity capture surface of a biochip of the present invention is provided, wherein the affinity capture monomers 6 associated with the first and second affinity capture zones 3 and 4 are further comprised of anchoring 6a, alkylene 6b, protein adsorption resistant 6c, cleavable 6d and affinity capture or (e.g., ligand or interactant) moieties 6e, wherein the hydrophilic-terminated monomers 7 associated with the first affinity capture zone 3 are further comprised of anchoring 7a, alkylene 7b, protein adsorption resistant 7c and hydrophilic moieties 7d, wherein the hydrophobic-terminated monomers 8 associated with the second affinity capture zone 4 are further comprised of anchoring 8a, alkylene 8b, protein adsorption resistant 8c and hydrophobic moieties 8d, and wherein the non-wettable monomers 9 associated with the common surrounding zone 5 are further comprised of anchoring 9a, alkylene 9b and perfluoroalkyl or perfluoroaryl moieties 9c.

With reference to FIG. 5, a cross-sectional view of the affinity capture surface of a biochip of the present invention is provided, wherein an aqueous liquid droplet 10 is localized on the surface of the biochip due to the presence of the non-wettable monomers associated with the common surrounding zone 5.

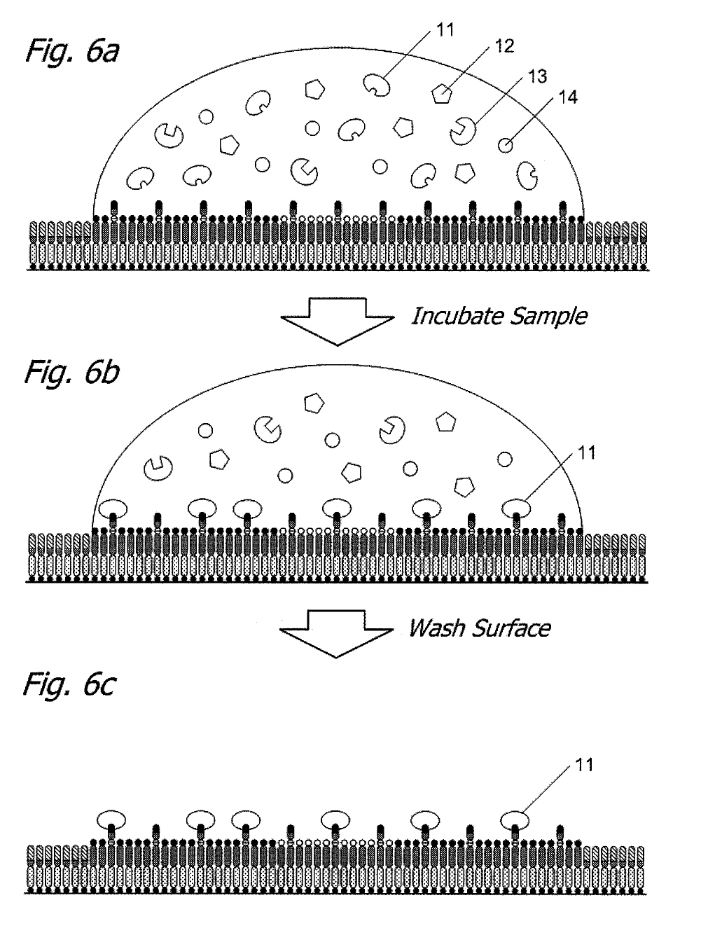

With reference to FIGS. 6a through 6c, the stepwise process for selective retention of a target analyte on the affinity capture surface of a biochip of the present invention is depicted. A liquid droplet containing the target analyte 11 as well as additional analytes 12, 13 and 14 is applied to the surface of the biochip and allowed to incubate for an appropriate period as determined by the nature of the interaction between the immobilized affinity capture or reactive moiety 6e (FIG. 6a). The surface of the biochip is next subjected to one or more washes to remove analytes exhibiting no affinity for the immobilized affinity capture or reactive moiety 6e (FIG. 6b). Finally, the wash solution is either removed or allowed to evaporate and the retained analyte remains of the surface of the biochip (FIG. 6e).

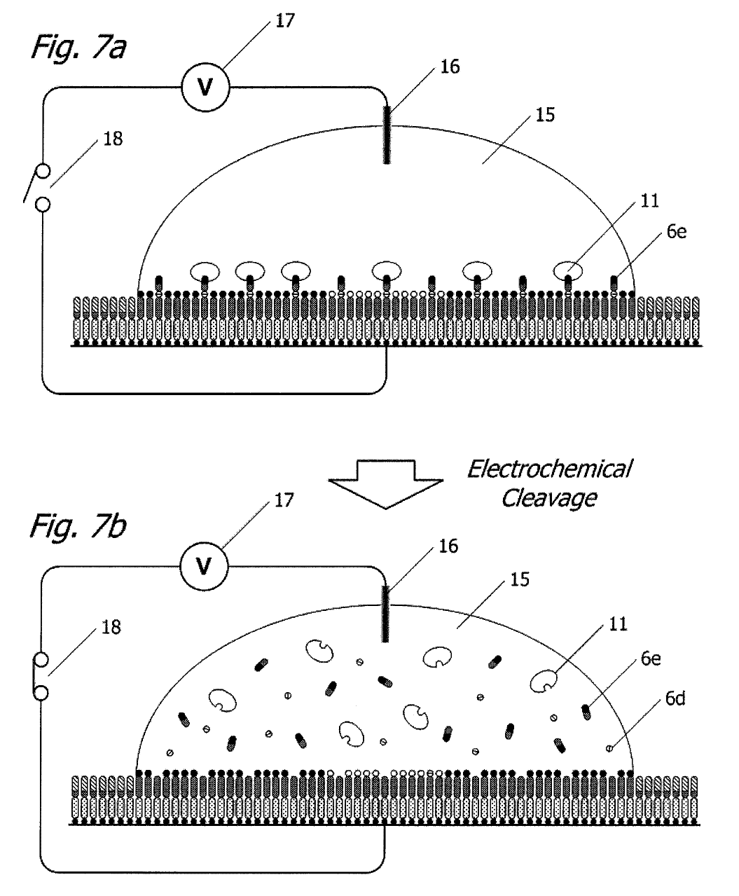

With reference to FIGS. 7a and 7b, in preparation for electrochemical cleavage of the affinity capture monomers associated with the first and second affinity capture zones, an anhydrous or aqueous electrolyte solution 15 is applied to the affinity capture surface of the biochip and a circuit comprised of, for example, a platinum counter-electrode 16, power supply 17 and a timer-controlled switch 18 is connected (FIG. 7a). A positive potential is applied to the affinity capture surface of the biochip resulting in electrochemical cleavage of the cleavable moiety 6d (FIG. 7b).

Representative anhydrous electrolytes include, but are not limited to, tetraethylammonium perchlorate, tetrabutylammonium perchlorate, tetraethylammonium tetrafluoroborate, tetrabutyl-ammonium tetrafluoroborate and triethylammonium acetate. Representative anhydrous solvents suitable for use in conjunction with anhydrous electrolytes include, but are not limited to, acetonitrile, dimethylformamide, ethanol, methanol and N-methylpyrrolidone. Representative aqueous electrolytes include, but are not limited to, ammonium perchlorate, perchloric acid, triethylammonium acetate and phosphate buffered saline.

In certain embodiments, electrochemical oxidation of the cleavable moieties on the affinity capture surface of the biochip is achieved by applying an potential of at most +1.6 V, for a period of from about 90 to 300 sec, when anhydrous electrolyte solutions are employed, and of at most +1.1 V, for a period of from about 90 to 300 sec, when aqueous electrolyte solutions are employed. All potentials are relative to the Ag/AgCl/KCl reference electrode. Oxidizing potentials may be applied either by application of a continuous fixed potential or by ramping from one potential to another at a fixed rate as in Cyclic Voltammetry.

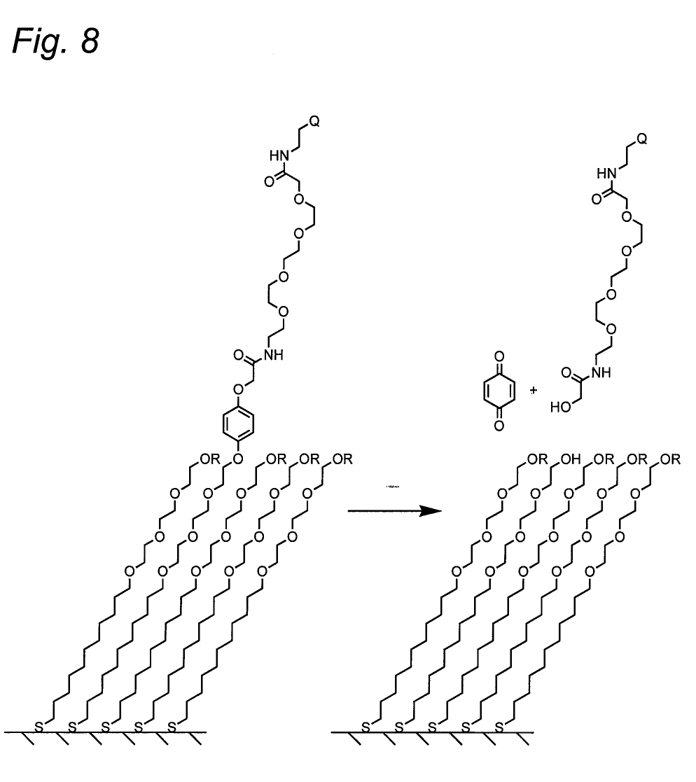

With reference to FIG. 8, a representative embodiment of the binary self-assembled monolayers associated with the first and second affinity capture zones is depicted, wherein “R” may be selected from, for example, one of H, CH₂CONH₂ and COCH₂OH when associated with the first affinity capture zone, and one of CH₃, COCH₃ and CH₂CH₃ when associated with the second affinity capture zone.

With reference to FIG. 8, cleavage of the quinone moiety under anhydrous conditions liberates the oxidized cleavable moiety as well as the pendant affinity capture or reactive moiety “Q” to yield a hydrophilic or hydrophobic surface depending upon the selection of “R”. In that the specific monomers from which the active moiety was cleaved can no longer be differentiated from the surrounding monomers, the cleavable linkage is said to be “traceless”. If a target analyte is retained on the surface of the biochip by interaction with the affinity capture or reactive moiety, it is concomitantly liberated at the time of electrochemical cleavage.

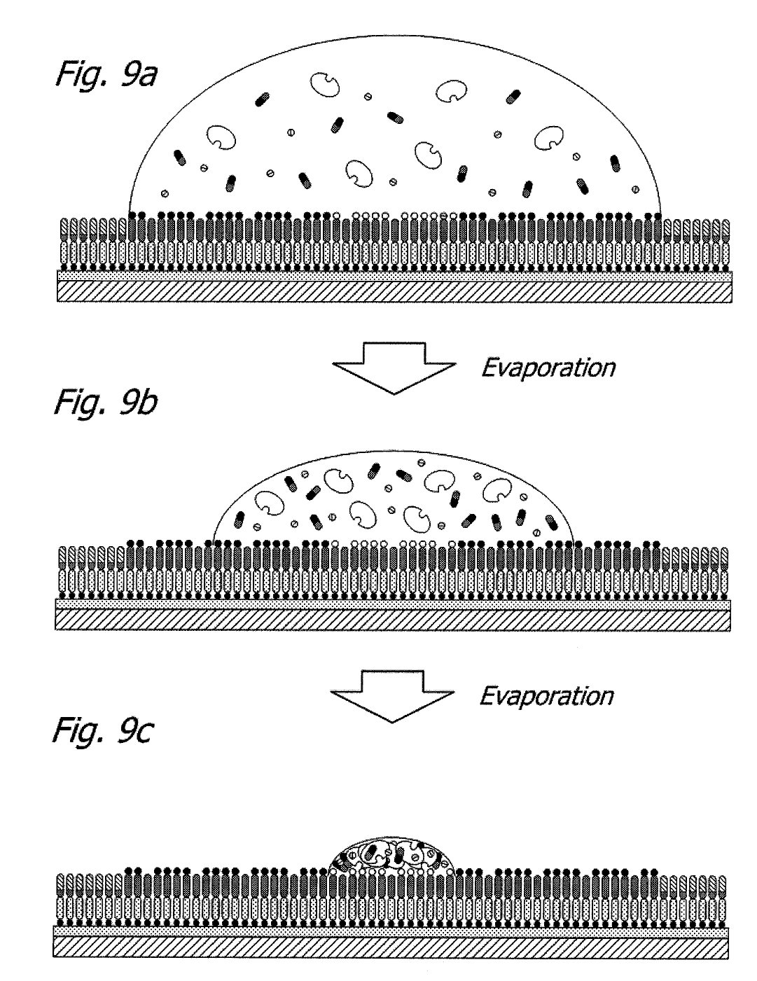

With reference to FIGS. 9a through 9c, the progressive evaporation of an analyte-containing liquid droplet resulting in localization of the analyte above the area corresponding to the first affinity capture zone is depicted. A volatile organic solvent containing less that 10% water is applied to the surface of the biochip resulting is dissolution of target analyte, affinity capture or reactive moiety, and cleavable moiety (FIG. 9a). Progressive evaporation of the volatile organic solvent results in a reduction in the volume of the droplet with a concomitant increase in the water-content of the droplet (FIG. 9b). Finally, the high-water-content evaporated liquid droplet interacts with the hydrophilic-terminated monomers associated with the first affinity capture zone resulting in localization of the target analyte above the area corresponding to the first affinity capture zone (FIG. 9c).

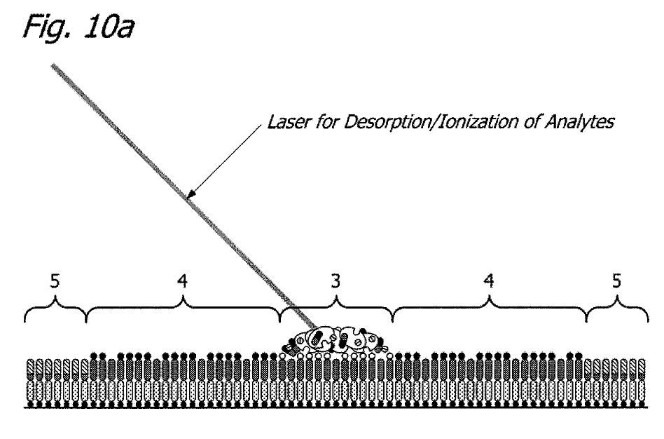

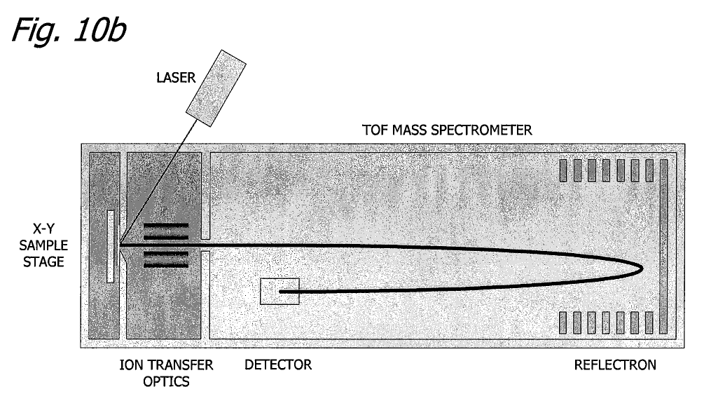

With reference to FIGS. 10a and 10b, the focused analyte on the surface of the biochip co-crystallized with an appropriate matrix compound as determined by the chemical classification of the analyte to be ionized to produce vaporous ions by matrix-assisted laser desorption/ionization in the inlet of the mass spectrometer is depicted.

### Affinity Capture Surface

In certain embodiments, the affinity capture monomers common to both the first and second affinity capture zones have one of the following general formulas:

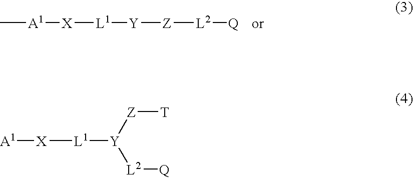

wherein:
A¹ is an anchoring moiety associated with the substrate surface;
X is alkylene;
L¹ is absent or is a protein adsorption resistant moiety;
Y is —O—Y¹, wherein Y¹ is aryl or substituted aryl, and wherein Y is bonded to L¹ and Z such that L¹ and Z are oriented in either an ortho or para relationship;
Z is a leaving group;
L² is absent or is a protein adsorption resistant moiety;
Q is an affinity capture moiety or reactive moiety; and
T is hydrogen, alkylene or aryl.

In more specific embodiments, the affinity capture monomers have the general formula:

-A¹-X-L¹-Y-Z-L²-Q (3)

wherein:
A¹ is —S— or —SCH₂CH₂CONH—;
X is C₃-C₁₈ alkylene;
L¹ is —(OCH₂CH₂)ₘ—, wherein m is an integer from 3 to 6;
Y is —O—Y¹, wherein Y¹ is aryl or substituted aryl, and wherein Y is bonded to L¹ and Z such that L¹ and Z are oriented in either an ortho or para relationship;
Z is —OCO—, —OCOO—, —OCONH—, —OSO₂—, —OPO₃—, —OCH₂COO—, —OCH₂CONH—, —OCH₂CH₂—, —OCH₂C₆H₄—, —OC₆H₄—, —OSi(CH₃)₂—, —OSi(CH₂CH₃)₂—, —OSi[CH(CH₃)₂]₂— or —OSi(C₆H₅)₂—;
L² is —(CH₂)ₙ— or —(CH₂CH₂O)ₙₙ—, wherein n an integer from 2 to 8 and nn is an integer from 2 to 6; and
Q is an affinity capture moiety or reactive moiety.

In yet more specific embodiments, the affinity capture monomers have the foregoing general formula (3), wherein:
A¹ is —S—;
X is —(CH₂)₁₁—;
L¹ is —(OCH₂CH₂)ₘ—, wherein m is 3 or 4;
Y is —O(C₆H₄)— and is bonded to L¹ and Z such that L¹ and Z are oriented in a para relationship;
Z is —OCH₂CONH—, —OCONH— or —OSi[CH(CH₃)₂]₂—;
L² is —(CH₂CH₂O)ₙₙ—, wherein nn is an integer from 2 to 6; and
Q is an affinity capture moiety or reactive moiety.

In certain embodiments, the hydrophilic-terminated monomers associated with the first affinity capture zone have the general formula:

-A¹-X-L¹-T¹

wherein:
A¹ is an anchoring moiety associated with the substrate surface;
X is alkylene;
L¹ is absent or is a protein adsorption resistant moiety; and
T¹ is a hydrophilic terminator.

In more specific embodiments, the hydrophilic-terminated monomers have the foregoing general formula, wherein:
A¹ is —S— or —SCH₂CH₂CONH—;
X is C₃-C₁₈ alkylene;
L¹ is —O(CH₂CH₂)ₘ—, wherein m is an integer from 3 to 6; and
T¹ is —OH and —OCH₂CONH₂, —OCONH₂ or —OCOCH₂OH.

In certain embodiments, the hydrophobic-terminated monomers associated with the second affinity capture zone have the general formula:

-A¹-X-L₁-T²

wherein:
A¹ is an anchoring moiety associated with the substrate surface;
X is alkylene;
L¹ is absent or is a protein adsorption resistant moiety; and
T² is a hydrophobic terminator.

In more specific embodiments, the hydrophobic-terminated monomers have the foregoing general formula, wherein:
A¹ is —S— or —SCH₂CH₂CONH—;
X is C₃-C₁₈ alkylene;
L¹ is —O(CH₂CH₂)ₘ—, wherein m is an integer from 3 to 6; and
T¹ is —OCH₃, —OCH₂CH₃, —OCOCH₃ and —OCOCF₃.

In certain embodiments, the non-wettable monomers associated with the common surrounding zone have the general formula:

-A¹-X-R

wherein:
A¹ is an anchoring moiety associated with the substrate surface;
X is alkylene; and
R contains a terminal perfluoroalkyl or perfluoroaryl moiety.

In more specific embodiments, the non-wettable monomers have the foregoing general formula, wherein:
A¹ is —S— or —SCH₂CH₂CONH—;
X is C₃-C₁₈ alkylene; and
R is —(CF₂)ᵣCF₃, —OCH₂CH₂(CF₂)ᵣCF₃ and —OCH₂C₆F₅, wherein r is an integer from 3 to 7.

The affinity capture moieties of group Q provide (1) sites for biological binding onto self-assembled monolayers prepared from the disclosed alkanethiols or (2) sites for physiochemical adsorption onto self-assembled monolayers prepared from the disclosed alkanethiols. In the first aspect (i.e., sites for biological binding), representative affinity capture moieties include, but are not limited to: any haptenic or antigenic compound in combination with the corresponding antibody or binding portion or fragment thereof (e.g., digoxigenin and anti-digoxigenin; fluorescein and anti-fluorescein; dinitrophenol and anti-dinitrophenol; bromodeoxyuridine and anti-bromodeoxyuridine); mouse immunoglobulin and goat anti-mouse immunoglobulin; nonimmunological binding pairs (e.g., biotin and avidin; biotin and streptavidin); hormone (e.g., thyroxine or cortisol) and hormone binding protein; receptor and receptor agonist or antagonist; immunoglobulin G and protein A; lectin and carbohydrate; enzyme and enzyme inhibitor; complementary polynucleotide pairs capable or forming nucleic acid duplexes or triplexes; and the like. Those of skill in the art will know of other biological binding pairs suitable for use in conjunction with the disclosed self-assembled monolayers. In the second aspect (i.e., sites for physiochemical adsorption), representative affinity capture moieties include, but are not limited to: trimethylammonium; diethylammonium; carboxylic acid; sulfonic acid; iminodiacetic acid; nitrilotriactetic acid; cyano; butyl; octyl; octadecyl; phenyl; and the like. Those of skill in the art of ion-exchange chromatography, metal ion affinity chromatography and hydrophobic interaction chromatography will know of the procedures utilized for selective adsorption of target molecules onto the disclosed self-assembled monolayers.

In certain embodiments, Q is a reactive moiety to which an affinity capture moiety is subsequently appended. Namely, the reactive moieties of group Q provide sites for the covalent immobilization of affinity capture moieties onto self-assembled monolayers prepared from the disclosed alkanethiols. Representative reactive moieties include, but are not limited to the following moieties: aldehyde; amino; bromoacetamido; carboxylic acid; chloroacetamido; dithiopyridyl; N-hydroxysuccinimidyl ester; imidazoyl; iodoacetamido; maleimide; pentafluororphenyl ester; pyridyldisulfide; thiol; and the like. Those of skill in the art of affinity chromatography will know of the procedures utilized to immobilize molecules onto the disclosed self-assembled monolayers.

In various embodiment, the substrate surface may comprise glass, metal, a polymeric material or silica an/or the surface may comprise gold or silver.

### Novel Alkanthiols and Self-Assembled Monolayers

The present invention also provides novel alkanethiols having one of the following general formulas:

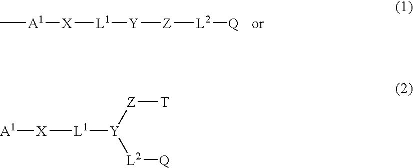

wherein:
A is an anchoring moiety;
X is alkylene;
L¹ is absent or is a protein adsorption resistant moiety;
Y is —O—Y¹, wherein Y¹ is aryl or substituted aryl, and wherein Y is bonded to L¹ and Z such that L¹ and Z are oriented in either an ortho or para relationship;
Z is a leaving group;
L² is absent or is a protein adsorption resistant moiety;
Q is an affinity capture moiety or reactive moiety; and
T is hydrogen, alkylene or aryl.

In more specific embodiments, the compound has the following general formula:

A-X-L¹-Y-Z-L²-Q (1)

wherein:
A is HS— or HSCH₂CH₂CONH—;
X is C₃-C₁₈ alkylene;
L¹ is —(OCH₂CH₂)ₘ—, wherein m is an integer from 3 to 6;
Y is —O—Y¹, wherein Y¹ is aryl or substituted aryl, and wherein Y is bonded to L¹ and Z such that L¹ and Z are oriented in either an ortho or para relationship;
Z is —OCO—, —OCOO—, —OCONH—, —OSO₂—, —OPO₃—, —OCH₂COO—, —OCH₂CONH—, —OCH₂CH₂—, —OCH₂C₆H₄—, —OC₆H₄—, —OSi(CH₃)₂—, —OSi(CH₂CH₃)₂—, —OSi[CH(CH₃)₂]₂— or —OSi(C₆H₅)₂—;
L² is —(CH₂)ₙ— or —(CH₂CH₂O)ₙₙ—, wherein n an integer from 2 to 8 and nn is an integer from 2 to 6; and
Q is an affinity capture moiety or reactive moiety.

In yet further more specific embodiments of the foregoing:
A is HS—;
X is —(CH₂)₁₁—;
L¹ is —(OCH₂CH₂)ₘ—, wherein m is 3 or 4;
Y is —O(C₆H₄)— and is bonded to L¹ and Z such that L¹ and Z are oriented in a para relationship;
Z is —OCH₂CONH—, —OCONH— or —OSi[CH(CH₃)₂]₂—;
L² is —(CH₂CH₂O)ₙₙ—, wherein nn is an integer from 2 to 6; and
Q is an affinity capture moiety or reactive moiety.

For example, the compound may have the following structure:

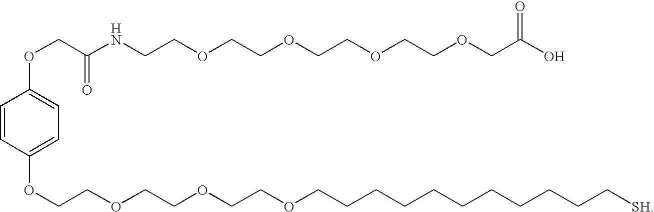

As one of skill in the art will appreciate, when contacted with a surface comprised of, e.g., gold, alkanethiols of foregoing general formula form self-assembled monolayers comprising surface coordinated moieties. More specifically, when A is a thiol moiety, during the course of the reaction, the hydrogen atom associated with the thiol moiety is lost, and the remaining —S— moiety is coordinated to the surface of the gold substrate. In this way, affinity capture surfaces (or self-assembled monolayers) may be prepared entirely from compounds and monomers having the foregoing general formulas, or may be prepared from binary mixtures of such compounds/monomers and additional hydrophobic-terminated and hydrophilic terminated compounds/monomers to yield the first and second binary self-assembled monolayers of the first and second affinity capture zones described above.

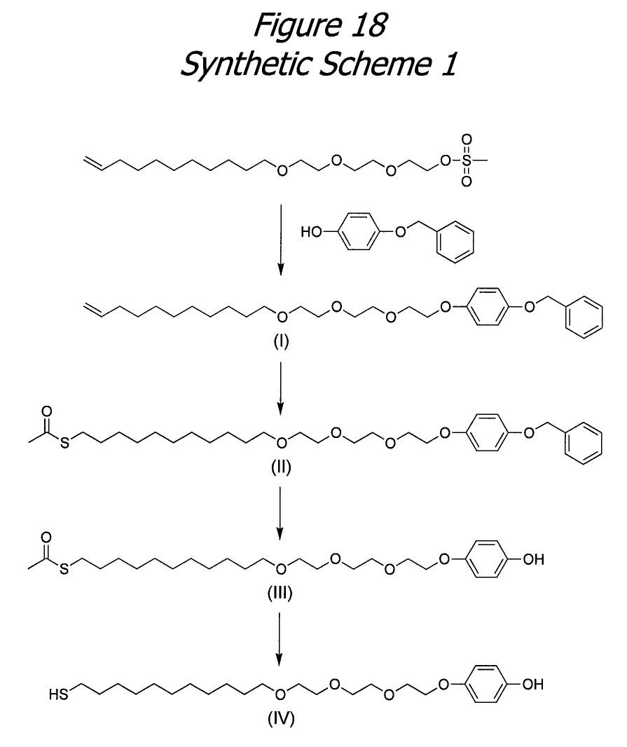

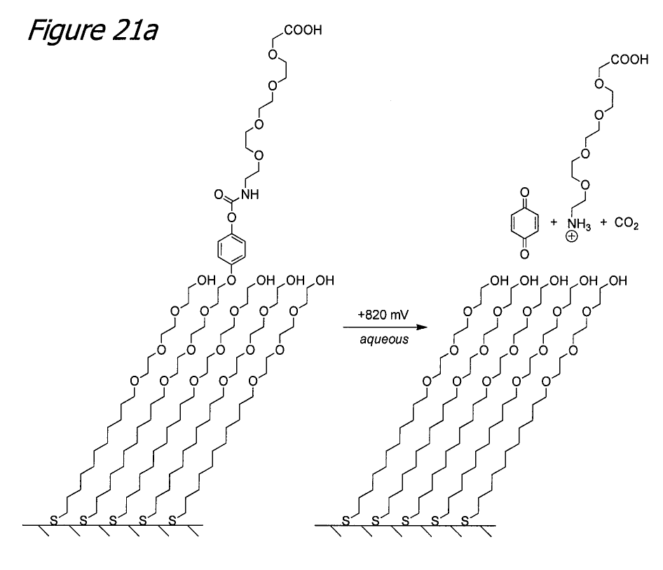

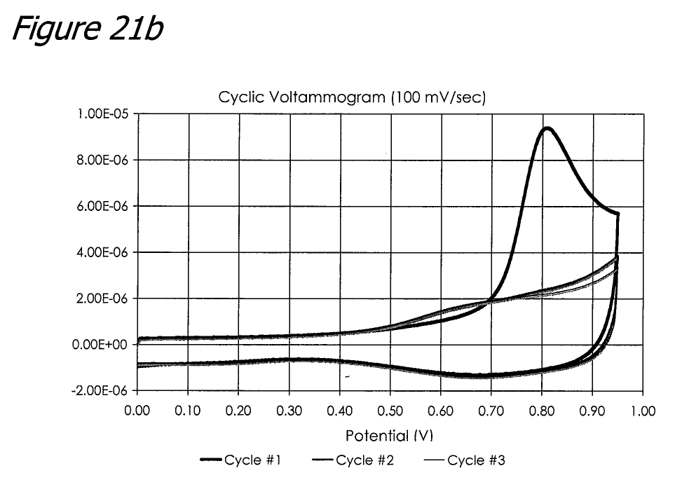

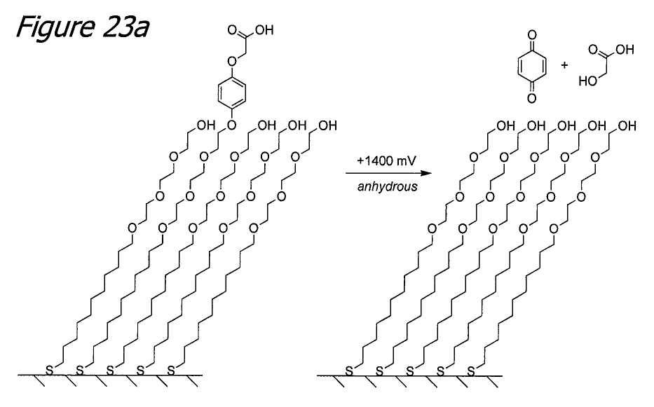

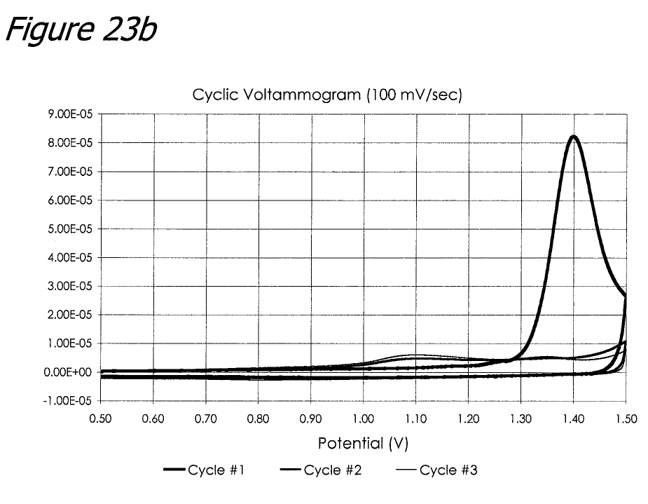

The disclosed alkanethiols may be synthesized using reagents and reactions well known to those of ordinary skill in the art, such as those described in “Advanced Organic Chemistry” J. March (Wiley & Sons, 1994); and “Organic Chemistry” Morrison and Boyd (Allyn and Bacon, 6th ed., 1992). In addition, for purposes of illustration, synthetic schemes related to various representative alkanethiols are depicted in, for example, FIGS. 18, 19, 20, 22, 24, 25, 26 and 27. For example, with reference to FIGS. 21a and 21b, the cleavage of a representative self-assembled monolayer, comprised of monomers of formula (XII) and 2-(2-(2-(11-mercaptoundecyloxy)ethoxy)ethoxy)ethanol is depicted in conjunction with the corresponding cyclic voltammogram. In this instance, the electrochemistry was undertaken under aqueous conditions. With reference to FIGS. 23a and 23b, the cleavage of a representative self-assembled monolayer, comprised of monomers of formula (XVIII) and 2-(2-(2-(11-mercaptoundecyloxy)ethoxy)ethoxy)ethanol is depicted in conjunction with the corresponding cyclic voltammogram. In this instance, the electrochemistry was undertaken under anhydrous conditions that enables the use of potentials that would be incompatible with self-assembled monolayers under aqueous conditions.

### Analyte-Containing Solution

The analyte-containing solution prepared according to the method of the present invention is comprised of target analyte(s) and terminal portions of the affinity capture monomers. Within the analyte-containing solution, the target analyte(s) and terminal portions of the affinity capture monomers may be either associated or disassociated. The stability of such complexes is influenced by the composition of the solution into which the complexes are released, including considerations such as pH, ionic strength, the presence of detergents and the presence of organic solvents.

Representative analytes include, but are not limited to: biological macromolecules such as peptides, proteins, enzymes, enzyme substrates, enzyme substrate analogs, enzyme inhibitors, polynucleotides, oligonucleotides, nucleic acids, carbohydrates, oligosaccharides, polysaccharides, avidin, streptavidin, lectins, pepstatin, protease inhibitors, protein A, agglutinin, heparin, protein G and concanavalin; fragments of biological macromolecules set forth above, such as nucleic acid fragments, peptide fragments and protein fragments; complexes of biological macromolecules set forth above, such as nucleic acid complexes, protein-DNA complexes, gene transcription complexes, gene translation complexes, membrane liposomes, membrane receptors, receptor ligand complexes, signaling pathway complexes, enzyme-substrate, enzyme inhibitors, peptide complexes, protein complexes, carbohydrate complexes and polysaccharide complexes; small biological molecules, such as amino acids, nucleotides, nucleosides, sugars, steroids, lipids, metal ions, drugs, hormones, amides, amines, carboxylic acids, vitamins and coenzymes, alcohols, aldehydes, ketones, fatty acids, porphyrins, carotenoids, plant growth regulators, phosphate esters and nucleoside diphosphosugars; synthetic small molecules, such as pharmaceutically or therapeutically effective agents, monomers, peptide analogs, steroid analogs, inhibitors, mutagens, carcinogens, antimitotic drugs, antibiotics, ionophores, antimetabolites, amino acid analogs, antibacterial agents, transport inhibitors, surface-active agents (surfactants), aminecontaining combinatorial libraries, dyes, toxins, biotin, biotinylated compounds, DNA, RNA, lysine, acetylglucosamine, procion red, glutathione, adenosine monophosphate, mitochondrial and chloroplast function inhibitors, electron donors, carriers and acceptors, synthetic substrates and analogs for proteases, substrates and analogs for phosphatascs, substrates and analogs for esterases and lipases and protein modification reagents; and synthetic polymers, oligomers, and copolymers, such as polyalkylenes, polyamides, poly(meth)acrylates, polysulfones, polystyrenes, polyethers, polyvinyl ethers, polyvinyl esters, polycarbonates, polyvinyl halides, polysiloxanes, and copolymers of any two or more of the above.

### Fabrication of Biochips

As noted above, the present invention provides a method of making the disclosed affinity capture surfaces, and biochips comprising such affinity capture surfaces, comprising the steps of: (a) providing a substrate surface (such as a conducting metal surface); (b) associating the second binary self-assembled monolayer (namely, the affinity capture monomers and the hydrophobic-terminated monomers) with the substrate surface; (c) ablating the second binary self-assembled monolayer from the substrate surface in the first affinity capture zone; and (d) associating the first binary self-assembled monolayer (namely, the affinity capture monomers and the hydrophilic-terminated monomers) with the ablated surface in the first affinity capture zone. In embodiments wherein the affinity capture surface further comprises a common surrounding zone, the method further comprises the steps (prior to associating the second binary self-assembled monolayer with the substrate surface): (a) associating the non-wettable monomers with the substrate surface; and (b) ablating the non-wettable self-assembled monolayer from the substrate surface in the first and second affinity capture zones.

In certain embodiments, the surface of a biochip of the present invention may be patterned by UV-photopatterning of self-assembled monolayers prepared from alkanethiols on gold or silver as described in U.S. Pat. No. 5,541,501, issued May 7, 1996, which is incorporated herein by reference in its entirety.

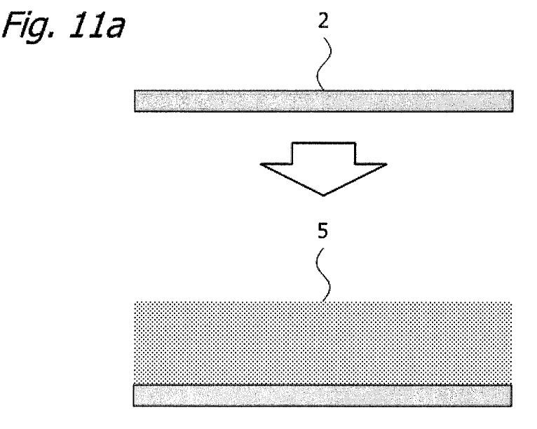

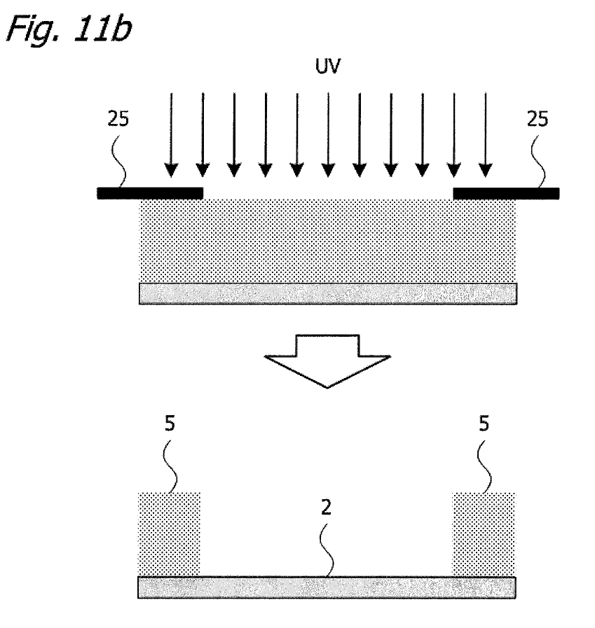

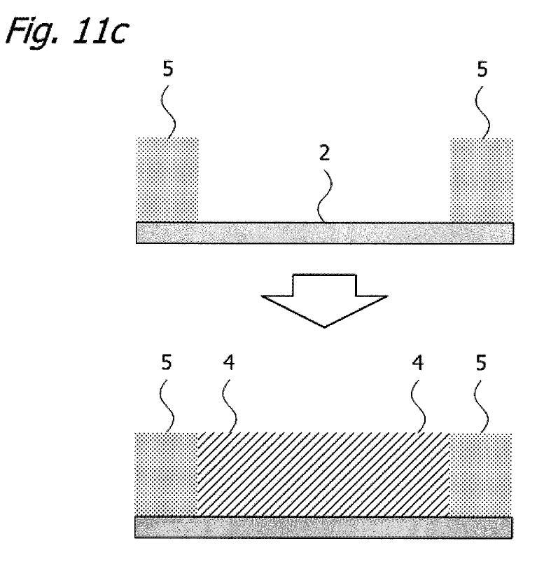

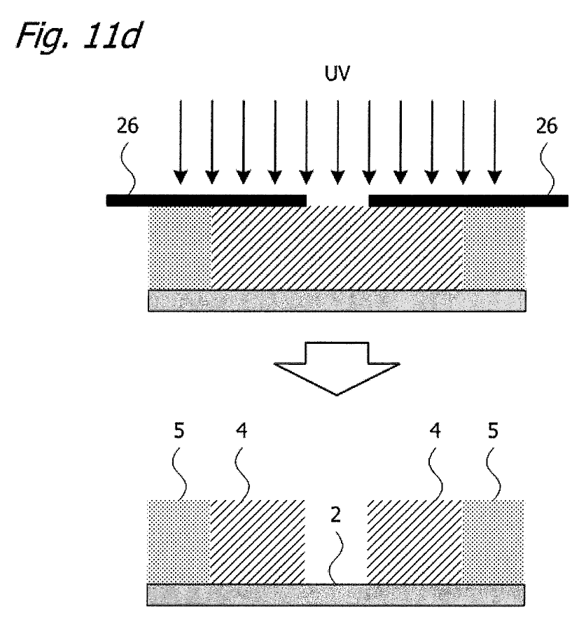

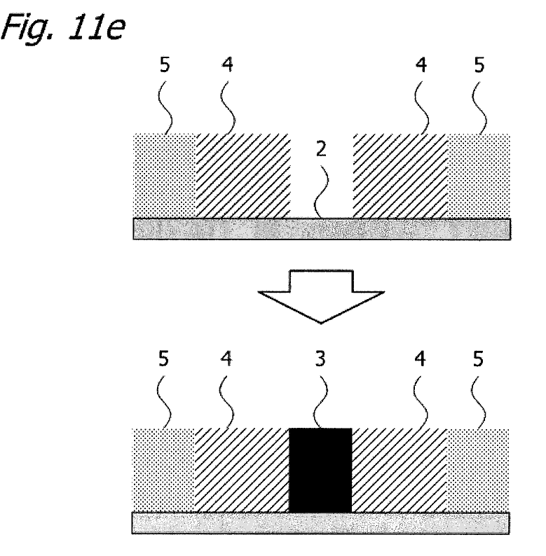

With reference to FIGS. 11a through 11e, a representative stepwise process for UV-photopatterning of self-assembled monolayers of alkanethiols on gold is depicted. Initially, a suitable biochip substrate such as a stainless steel plate is cleaned by wet chemical and plasma processes to remove surface contamination. An adhesion layer of titanium and tungsten (9:1) having a thickness of from about 200 to 500 angstroms is first applied to the surface followed by a layer of gold having a thickness of from about 1000 to 3000 angstroms. Metal deposition is accomplished by sputtering (vapor deposition) of gold by a calibrated process that provides a know thickness of metal per unit time.

With reference to FIG. 11a, the non-wettable common surrounding zone self-assembled monolayer 5 is assembled on the gold surface 2 by passive adsorption of monomer from ethanol solution for a period of from about 1 to 2 hours. The surface modified substrate is washed with ethanol to remove excess alkanethiol and dried under a stream of nitrogen.

With reference to FIG. 11b, the surface modified substrate is photopatterned by exposure to an intense UV light source through a first etched stainless steel shadow mask 25 having a thickness of from about 0.02 to 0.03 inch. Preferably, the intensity of the UV light source is in the range of from about 0.5 to 3 watts/cm². Total exposure time of from about 30 to 60 min. in the presence of oxygen is required to ablate the self-assembled monomer in the exposed area by oxidation of surface-coordinated monomer gold-thiolates to the corresponding sulfonates that exhibit limited affinity for the gold surface. The opening in the shadow mask 25 defines the diameter of the second affinity capture zone.

With reference to FIG. 11c, subsequent washing of the gold surface with ethanol removes monomer sulfonates and affords an unmodified region of gold 2. The second binary self-assembled monolayer 4, comprised of affinity capture monomers and hydrophobic-terminated monomers and corresponding to the second affinity capture zone, is assembled on the exposed gold surface by passive adsorption of monomers from ethanol solution for a period of from about 8 to 24 hours. The surface modified substrate is washed with ethanol to remove excess alkanethiol and dried under a stream of nitrogen.

With reference of FIG. 1d the once-patterned substrate is further photo-patterned by exposure to an intense UV light source through a second etched stainless steel shadow mask 26. Total exposure time of from about 30 to 60 min. in the presence of oxygen is required to ablate the self-assembled monomer in the exposed area by oxidation of surface-coordinated monomer gold-thiolates to the corresponding sulfonates that exhibit limited affinity for the gold surface. The opening in the shadow mask 26 defines the diameter of the first affinity capture zone.

With reference to FIG. 1e, subsequent washing of the gold surface with ethanol removes monomer sulfonates and affords an unmodified region of gold 2. The first binary self-assembled monolayer 3 comprised of affinity capture monomers and hydrophilic-terminated monomers and corresponding to the first affinity capture zone, is assembled on the exposed gold surface by passive adsorption of monomers from ethanol solution for a period of from about 8 to 24 hours. The surface modified substrate is washed with ethanol to remove excess alkanethiol and dried under a stream of nitrogen.

In this manner, the step-wise process for the UV-photopatterning of self-assembled monolayers prepared from alkanethiols on gold may be exploited to prepare biochips of the present invention.

The first binary self-assembled monolayer associated with the first affinity capture zone may prepared from the affinity capture monomers of general formulas (3) and (4) and the hydrophilic-terminated monomers described above. Similarly, the second binary self-assembled monolayer associated with the second affinity capture zone may be prepared from the affinity capture monomers of general formulas (3) and (4) and the hydrophobic-terminated monomers described above. The non-wettable self-assembled monolayer associated with the non-wettable common surrounding zone may be prepared from non-wettable monomers described above.

The first binary self-assembled monolayer associated with the first affinity capture zone may be prepared by passive adsorption from ethanol solution containing from about 0.01 mM to 1 mM total monomer (affinity capture monomers+hydrophilic-terminated monomers) for a period of from about 8 to 24 hours. Similarly, the second binary self-assembled monolayer associated with the second affinity capture zone may be prepared by passive adsorption from ethanol solution containing from about 0.01 mM to 1 mM total monomer (affinity capture monomers+hydrophobic-terminated monomers) for a period of from about 8 to 24 hours. The non-wettable self-assembled monolayer associated with the common surrounding zone may be prepared by passive adsorption from ethanol containing from about 0.01 to 1 mM monomer (non-wettable monomers) for a period of from about 1 to 2 hours.

In certain embodiments, the binary self-assembled monolayers associated with both the first and second affinity capture zones are prepared from at most 20% of the affinity capture monomers and 80% of the hydrophilic-terminated or hydrophobic-terminated monomers, respectively. In other embodiments, the binary self-assembled monolayers associated with both the first and second affinity capture zones are prepared from at most 10% of the affinity capture monomers and 90% of the hydrophilic-terminated or hydrophobic-terminated monomers, respectively. In yet other embodiments, the binary self-assembled monolayers associated with both the first and second affinity capture zones are prepared from 5% of the affinity capture monomers and 95% of the hydrophilic-terminated or hydrophobic-terminated monomers, respectively.

Alternatively, the binary self-assembled monolayers associated with the affinity capture zones of the present invention, as well as the self-assembled monolayer associated with the common surrounding zone of the present invention, may be prepared by potential-assisted deposition from ethanol containing from about 0.001 mM to 0.1 mM total monomer in conjunction with an applied potential of from about +200 to +400 mV on the gold surface of the biochip for a period of from about 5 to 15 min.

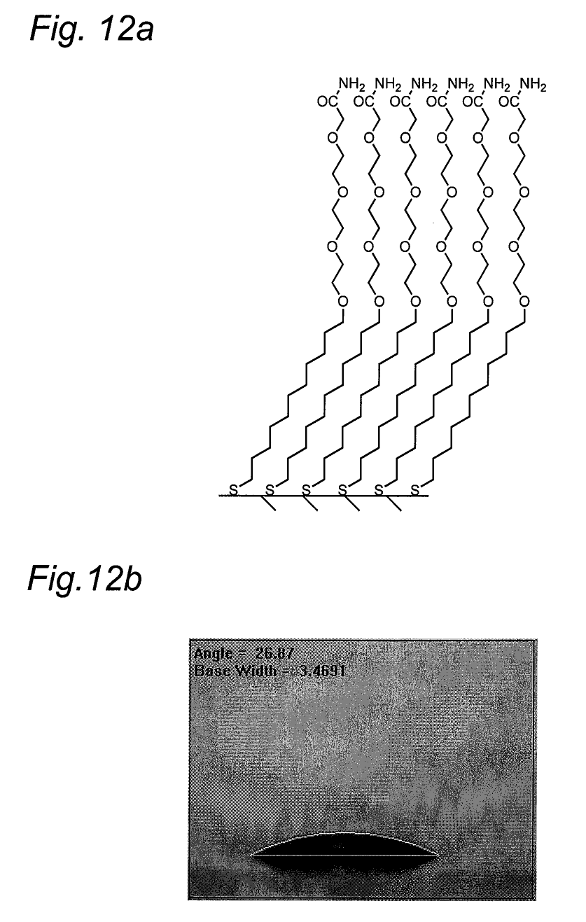

With reference to FIGS. 12a and 12b, a self-assembled monolayer comprised of hydrophilic-terminated monomers suitable for use in conjunction with the first affinity capture zone of a biochip of the present invention is depicted (FIG. 12a). Also depicted is an image of a water droplet (2.0 microliters) applied to the surface of the aforementioned self-assembled monolayer prepared on gold (FIG. 12b). Note that the surface is effectively wetted (hydrophilic) due to the presence of the hydrophilic-terminated monomers.

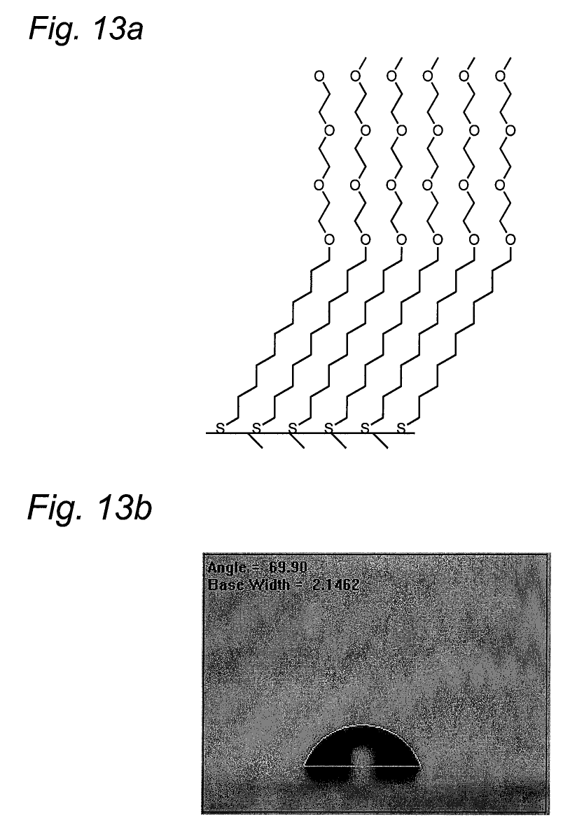

With reference to FIGS. 13a and 13b, a self-assembled monolayer comprised of hydrophobic-terminated monomers suitable for use in conjunction with the second affinity capture zone of a biochip of the present invention is depicted (FIG. 13a). Also depicted is an image of a water droplet (2.0 microliters) applied to the surface of the aforementioned self-assembled monolayer prepared on gold (FIG. 13b). Note that the surface is not effectively wetted (moderately hydrophobic) due to the presence of the hydrophobic-terminated monomers.

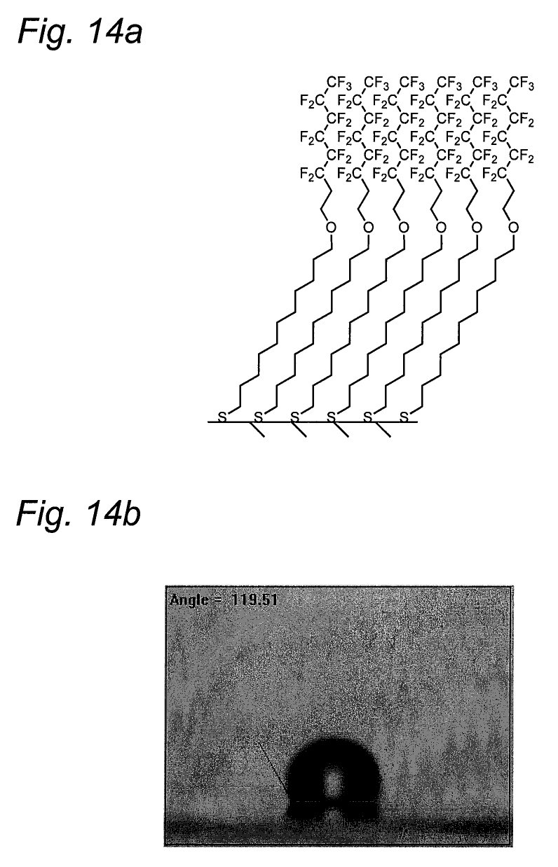

With reference to FIGS. 14a and 14b, a self-assembled monolayer comprised of non-wettable monomers suitable for use in conjunction with the common surrounding zone of a biochip of the present invention is depicted (FIG. 14a). Also depicted is an image of a water droplet (2.0 microliters) applied to the surface of the aforementioned self-assembled monolayer prepared on gold (FIG. 14b). Note that the surface is not wetted (extremely hydrophobic) due to the presence of the perfluoroalkyl-terminated monomers.

The following examples have been included to illustrate certain embodiments and aspects of the present invention, and should not be construed as limiting in any way.

### EXAMPLES

### Example 1

### UV-Photopatterning of Antibody Immobilization Biochips

Gold-on-glass substrates were placed in the chamber of the UV/Ozone cleaning apparatus, irradiated for 15 min and allowed to incubate in the presence of ozone for a further 15 min. The substrates were again irradiated for 15 min and allowed to incubate in the presence of ozone for a further 15 min. The substrates were placed in a Teflon® carrier, submerged into a 250 mL beaker containing 150 mL of ethanol, washed with continuous stirring for 30 min.

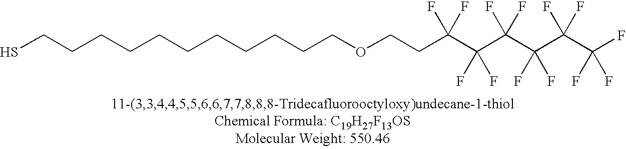

The substrates in a Teflon® carrier were next submerged into a 250 mL beaker containing 150 mL of 0.1 mM 11-(3,3,4,4,5,5,6,6,7,7,8,8,8-tridecafluorooctyloxy)-undecane-1-thiol in ethanol. The beaker was covered with plastic film that was secured with a rubber band and stirred continuously for 2 hours. The surface-modified substrates in the Teflon® carrier were removed from the reaction beaker and transferred into a 250 mL beaker containing 150 mL of ethanol. The substrates were washed with continuous stirring for 30 min, and then individually transferred to centrifugal washer, rinsed with ethanol for 1 min at 2500 rpm and then allowed to dry for 30 sec at 2500 rpm.

Surface-modified substrates prepared as described above were fitted with etched stainless steel shadow masks (0.02 inch thickness) having an array (8×8) of 3.0 mm diameter openings on 5.080 mm centers. The masks were held in place by pairs of ⅜″ Kapton® dots. The substrates were placed in the chamber of the UV/Ozone cleaning apparatus and the illumination chamber of the apparatus placed on vertical offsets (1.0 cm) to prevent the accumulation of ozone in the chamber. Surface-modified substrates were irradiated through shadow masks four times for 15 min each. Upon completion of each 15 min period, the chamber was allowed to cool for 15 min (to room temperature) before the next period of irradiation was commenced. After 30 min of irradiation, the substrates located in other than the central position were rotated 180° to ensure even illumination. After 60 min of irradiation, the chamber was allowed to cool and the substrates removed. The shadow masks were removed and the substrates placed in a Teflon® carrier, submerged into a 250 mL beaker containing 150 mL of ethanol and washed with continuous stirring for 30 min.

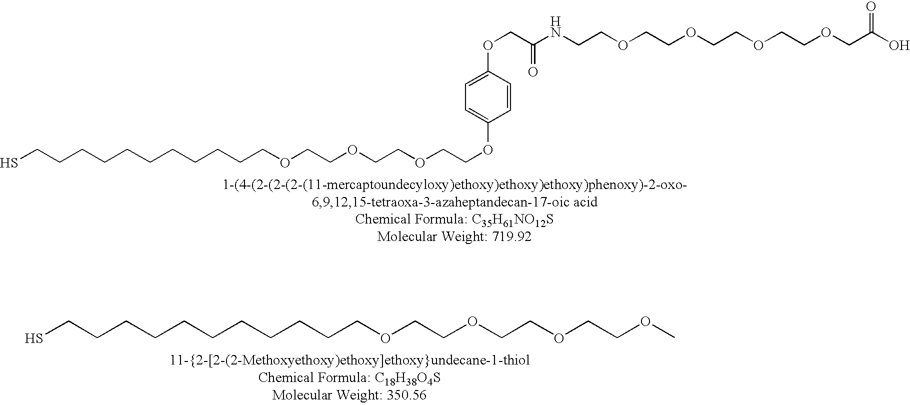

UV-photopatterned substrates were placed in a Teflon® carrier and submerged into a 250 mL beaker containing 150 mL of 0.005 mM 1-(4-(2-(2-(2-(11-mercaptoundecyloxy)ethoxy)ethoxy)ethoxy)phenoxy)-2-oxo-6,9,12,15-tetraoxa-3-aza-heptadecan-17-oic acid and 0.095 mM 11-{2-[2-(2-mercaptoethoxy)ethoxy]ethoxy}-undecane-1-thiol in ethanol. The beaker was covered with plastic film that was secured with a rubber band and stirred continuously overnight. Surface-modified substrates in the Teflon® carrier were removed from the reaction beaker and transferred into a 250 mL beaker containing 150 mL of ethanol. The substrates were washed with continuous stirring for 30 min, and then individually transferred to centrifugal washer, rinsed with ethanol for 1 min at 2500 rpm and then allowed to dry for 30 sec at 2500 rpm.

Surface-modified substrates (2×) prepared as described above were fitted with etched stainless steel shadow masks (0.02 inch thickness) having an array (8×8) of 0.6 mm diameter openings on 5.080 mm centers. The masks were held in place by pairs of ⅜″ Kapton® dots. The substrates were placed in the chamber of the UV/Ozone cleaning apparatus and the illumination chamber of the apparatus placed on vertical offsets (1.0 cm) to prevent the accumulation of ozone in the chamber. Surface-modified substrates were irradiated through shadow masks four times for 15 min each. Upon completion of each 15 min period, the chamber was allowed to cool for 15 min (to room temperature) before the next period of irradiation was commenced. After 30 min of irradiation, the substrates located in other than the central position were rotated 180° to ensure even illumination. After 60 min of irradiation, the chamber was allowed to cool and the substrates removed. The shadow masks were removed and the substrates placed in a Teflon® carrier, submerged into a 250 mL beaker containing 150 mL of ethanol and washed with continuous stirring for 30 min.

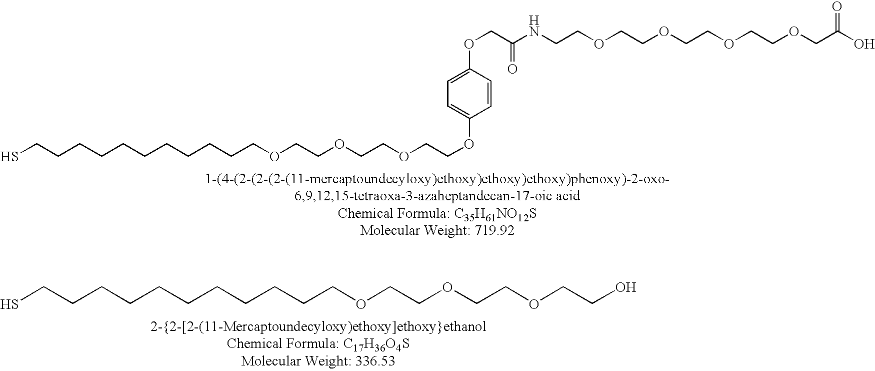

UV-photopatterned substrates (2×) were placed in a Teflon® carrier and submerged into a 250 mL beaker containing 150 mL of 0.005 mM 1-(4-(2-(2-(2-(11-mercaptoundecyloxy)-ethoxy)ethoxy)ethoxy)phenoxy)-2-oxo-6,9,12,15-tetraoxa-3-aza-heptadecan-17-oic acid and 0.095 mM 2-{2-[2-(11-mercaptoundecyloxy)ethoxy]ethoxy}ethoxyethanol in ethanol. The beaker was covered with plastic film that was secured with a rubber band and stirred continuously overnight. The surface-modified substrates (3×) in the Teflon® carrier were removed from the reaction beaker and transferred into a 250 mL beaker containing 150 mL of ethanol. The substrates were washed with continuous stirring for 30 min, and then individually transferred to centrifugal washer, rinsed with ethanol for 1 min at 2500 rpm and then allowed to dry for 30 sec at 2500 rpm. The resulting biochips were individually stored in polypropylene cases in a vacuum desiccator.

### Example 2

### UV-Photopatterning of Biotin Biochips with Negative Photomasks

Gold-on-glass substrates were placed in the chamber of the UV/Ozone cleaning apparatus, irradiated for 15 min and allowed to incubate in the presence of ozone for a further 15 min. The substrates were again irradiated for 15 min and allowed to incubate in the presence of ozone for a further 15 min. The substrates were placed in a Teflon® carrier, submerged into a 250 mL beaker containing 150 mL of ethanol, washed with continuous stirring for 30 min.

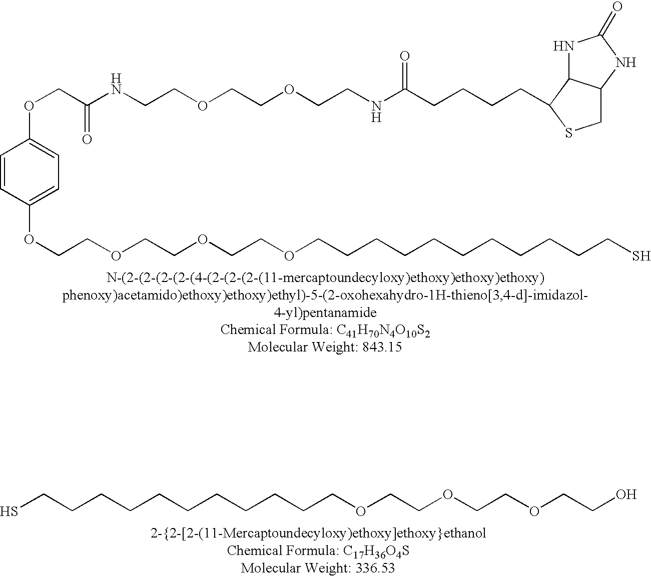

The substrates in the Teflon® carrier were next submerged into a 250 mL beaker containing 150 mL of 0.01 mM N-(2-(2-(2-(2-(4-(2-(2-(2-(11-mercaptoundecyloxy)ethoxy)-ethoxy)ethoxy)phenoxy)acetamido)ethoxy)ethoxy)ethyl)-5-(2-oxo-hexahydro-1H-thieno[3,4-d]-imidazol-4-yl)pentanamide and 0.09 mM 2-{2-[2-(11-mercapto-undecyloxy)ethoxy]ethoxy}-ethanol in ethanol. The beaker was covered with plastic film that was secured with a rubber band and stirred continuously overnight. The surface-modified substrates in the Teflon® carrier were removed from the reaction beaker and transferred into a 250 mL beaker containing 150 mL of ethanol. The substrates were washed with continuous stirring for 30 min, and then individually transferred to centrifugal washer, rinsed with ethanol for 1 min at 2500 rpm and then allowed to dry for 30 sec at 2500 rpm.

Surface-modified substrates prepared as described above were individually fitted with quartz photomasks (1.5 mm thickness) having arrays (8×8) of 0.6 mm diameter chrome oxide spots on 5.080 mm centers. The masks were affixed to the substrates with pairs of ⅜″ Kapton® dots with the metal oxide surface of the mask in proximity to the gold surface of the substrate. The masks were held in place by pairs of ⅜″ Kapton® dots. The substrates were placed in the chamber of the UV/Ozone cleaning apparatus and the illumination chamber of the apparatus placed on vertical offsets (1.0 cm) to prevent the accumulation of ozone in the chamber. Surface-modified substrates were irradiated through shadow masks four times for 15 min each. Upon completion of each 15 min period, the chamber was allowed to cool for 15 min (to room temperature) before the next period of irradiation was commenced. After 30 min of irradiation, the substrates located in other than the central position were rotated 180° to ensure even illumination. After 60 min of irradiation, the chamber was allowed to cool and the substrates removed. The photomasks were removed and the substrates placed in a Teflon® carrier, submerged into a 250 mL beaker containing 150 mL of ethanol and washed with continuous stirring for 30 min.

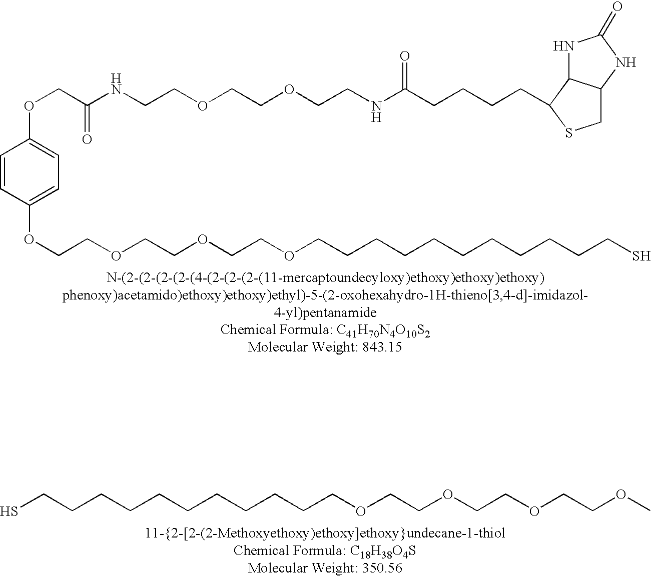

UV-photopatterned substrates were placed in a Teflon® carrier and submerged into a 250 mL beaker containing 150 mL of 0.01 mM N-(2-(2-(2-(2-(4-(2-(2-(2-(11-mercaptoundecyl-oxy)ethoxy)ethoxy)ethoxy)phenoxy)acetamido)ethoxy)ethoxy)ethyl)-5-(2-oxohexahydro-1H-thieno[3,4-d]-imidazol-4-yl)pentanamide and 0.09 mM 11-{2-[2-(2-mercaptoethoxy)ethoxy]-ethoxy}undecane-1-thiol in ethanol. The beaker was covered with plastic film that was secured with a rubber band and stirred continuously overnight. Surface-modified substrates in the Teflon® carrier were removed from the reaction beaker and transferred into a 250 mL beaker containing 150 mL of ethanol. The substrates were washed with continuous stirring for 30 min, and then individually transferred to centrifugal washer, rinsed with ethanol for 1 min at 2500 rpm and then allowed to dry for 30 sec at 2500 rpm.

Surface-modified substrates (2×) prepared as described above were individually fitted with quartz photomasks (1.5 mm thickness) having arrays (8×8) of 3.3 mm diameter chrome oxide spots on 5.080 mm centers. The masks were affixed to the substrates with pairs of ⅜″ Kapton® dots with the metal oxide surface of the mask in proximity to the gold surface of the substrate. The masks were held in place by pairs of ⅜″ Kapton® dots. The substrates were placed in the chamber of the UV/Ozone cleaning apparatus and the illumination chamber of the apparatus placed on vertical offsets (1.0 cm) to prevent the accumulation of ozone in the chamber. Surface-modified substrates were irradiated through shadow masks four times for 15 min each. Upon completion of each 15 min period, the chamber was allowed to cool for 15 min (to room temperature) before the next period of irradiation was commenced. After 30 min of irradiation, the substrates located in other than the central position were rotated 180° to ensure even illumination. After 60 min of irradiation, the chamber was allowed to cool and the substrates removed. The photomasks were removed and the substrates placed in a Teflon® carrier, submerged into a 250 mL beaker containing 150 mL of ethanol and washed with continuous stirring for 30 min.

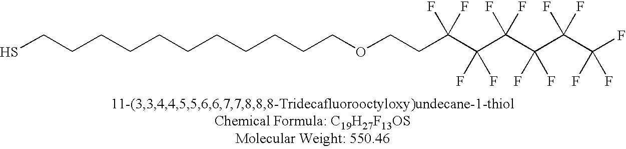

UV-photopatterned substrates (2×) were placed in a Teflon® carrier and submerged into a 250 mL beaker containing 150 mL of 0.1 mM 11-(3,3,4,4,5,5,6,6,7,7,8,8,8-tridecafluorooctyl-oxy)undecane-1-thiol in ethanol. The beaker was covered with plastic film that was secured with a rubber band and stirred continuously overnight. The surface-modified substrates (3×) in the Teflon® carrier were removed from the reaction beaker and transferred into a 250 mL beaker containing 150 mL of ethanol. The substrates were washed with continuous stirring for 30 min, and then individually transferred to centrifugal washer, rinsed with ethanol for 1 min at 2500 rpm and then allowed to dry for 30 sec at 2500 rpm. The resulting biochips were individually stored in polypropylene cases in a vacuum desiccator.

### Example 3

### Immobilization of Antibodies on Biochips

Three approaches to antibody immobilization were compared to determine the optimum methodology with respect to immobilization of anti-Human Transthyretin. Six biochips prepared as described in Example 1, comprised of 5% carboxylic acid-terminated monomer in both the first and second affinity capture zones, were individually conditioned with 25 mL of 25 mM Sodium Phosphate Buffer, pH 8.0 on an end-over-end shaker for 30 min. Droplets were shaken from the surfaces of the biochips and the surfaces blotted dry with Kimwipes®.

### Surface Activation

Surface activation solution (3.0 mL) comprised of 0.2 M N-(3-dimethylaminopropyl)-N′-ethylcarbodiimide hydrochloride and 0.05 M N-hydroxysuccinimide was freshly prepared with 18 MΩ water in a 15 mL conical test tube. Three pairs of conditioned biochips were placed in 500 mL polycarbonate Nalgene® jars on top of damp sponges and 10.0 μL aliquots of activation solution added to each of 48 sites per biochip. Tops were placed on the jars and the biochips incubated for 90 min. Finally, biochips were washed with 18 MΩ water from a wash bottle, the surfaces rinsed with acetone and dried under a stream of nitrogen.

### Immobilization of Protein A, Protein G and Anti-Human Transthyretin

Three pairs of carbodiimide-activated biochips were placed in 500 mL polycarbonate Nalgene® jars on top of damp sponges. Aliquots of protein A (5.0 μL at 0.5 μg/mL) in PBS Buffer, pH 7.2 were added to each of 16 sites per biochip (A5-D8), aliquots of protein G (5.0 μL at 0.5 μg/mL) in PBS Buffer, pH 7.2 were added to each of 16 sites per biochip (E1-H4), and aliquots of rabbit polyclonal anti-Human Transthyretin (5.0 μL at 1.0 μg/mL) in PBS Buffer, pH 7.2 were added to each of 16 sites per biochip (E5-H8). Finally, tops were placed on the jars and the biochips incubated overnight at room temperature. Aliquots were shaken from the surfaces and the biochips individually washed once with 25 mL of PBS Buffer, pH 7.2 with 0.5% Triton X-100 for 30 min and twice with 25 mL of PBS Buffer, pH 7.2 for 15 min on an end-over-end shaker. Finally, the biochip surfaces were blotted dry with Kimwipes®.

Biochips prepared as described immediately above were placed as pairs in 500 mL polycarbonate Nalgene® jars on top of damp sponges and 5.0 μL aliquots of 50 mM ethanolamine-0.1% Tween-20, pH 8.0 was applied to each of 48 sites per biochip to block any residual carbodiimide-activated sites. Tops were placed on the jars and the biochips incubated for 30 min at room temperature. Aliquots were shaken from the surfaces and the biochips individually washed once with 25 mL of PBS Buffer, pH 7.2 with 0.5% Triton X-100 for 30 min and twice with 25 mL of PBS Buffer, pH 7.2 for 15 min on an end-over-end shaker. Finally, the biochip surfaces were blotted dry with Kimwipes®.

### Antibody Immobilization on Protein A and Protein G

A 5.0 μL aliquot of anti-Human Transthyretin at 2.0 μg/mL in PBS buffer, pH 7.2 was added to each of 32 sites per biochip corresponding to immobilized protein A and protein G. Top were placed on the jars and the biochips incubated for 90 min at room temperature. Aliquots were shaken from the surfaces of the biochips and the biochips individually washed once with 25 mL of PBS Buffer, pH 7.2 with 0.5% Triton X-100 for 30 min and twice with 25 mL of PBS Buffer, pH 7.2 for 15 min on an end-over-end shaker. Finally, the biochip surfaces were blotted dry with Kimwipes®.

### Immobilized Antibody Capture of Transthyretin

Fifty microliter aliquots of four human serum samples were added to four 1.5 mL centrifuge tubes, diluted 1:1 with PBS Buffer, mixed by vortexing and centrifuged at 6000 rpm for 3 min to remove particulates. Two biochips, prepared as described above, were placed as a pair in a 500 mL polycarbonate Nalgene® jar on top of damp sponge. Ten microliter aliquots of each of the human serum sample supernatants were applied to each of 12 sites per biochip corresponding to: protein A/anti-Human Transthyretin (4 sites); protein G/anti-Human Transthyretin (4 sites); and Transthyretin (4 sites). The top was placed on the jar and the biochips incubated for 90 min at room temperature. Aliquots were shaken from the surfaces and the biochips individually washed once with 25 mL of PBS Buffer, pH 7.2 with 0.5% Triton X-100 for 30 min and twice with 25 mL of PBS Buffer, pH 7.2 for 15 min on an end-over-end shaker. Finally, the biochip surfaces were rinsed with 18 MΩ water from a wash bottle and blotted dry with Kimwipes®.

### MALDI-Mass Spectrometry

A matrix stock solution was prepared by dissolving 2.0 mg of sinapinic acid in 1.0 mL of acetonitrile and 0.1% trifluoroacetic acid (6:4) in a 1.5 mL microcentrifuge tube by vortexing. The solution was centrifuged at 1500 rpm for 1.5 min and the supernatant transferred to a labeled 1.5 mL microcentrifuge tube. An aliquot of matrix solution (2.0 μL) was applied to each of 12 sites per biochip corresponding to the serum sample sites, and an aliquot (2.0 μL) of matrix solution containing Cytochrome c (50 fmol/μL) was applied to each of 4 sites per biochip (molecular weight calibration standard). Matrix was allowed to crystallize for 1 hr at room temperature as the process was monitored under a stereomicroscope.

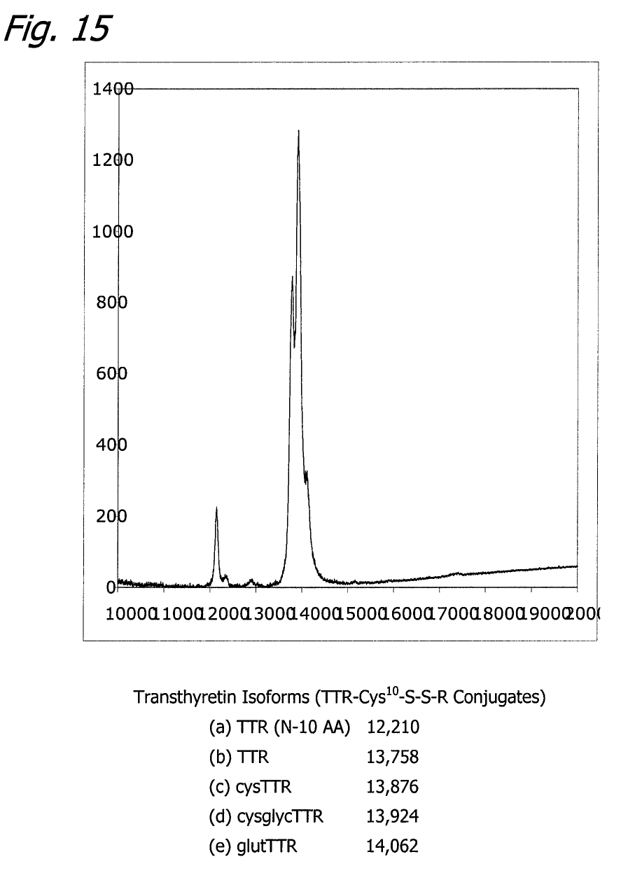

Each biochip was placed in stainless steel carrier, inserted into a Voyager DE MALDI-TOF-MS, and the instrument calibrated with respect to Cytochome c (external standard) in the positive ion-linear mode. Each sample site was subsequently analyzed by averaging the data corresponding to 20 laser shots at locations determined to be suitable for data acquisition. Analysis of the data revealed the following the order with respect to recovery of native Transthyretin from human serum: protein G/anti-Human Transthyretin>protein A/anti-Human Transthyretin>anti-Human Transthyretin. With reference to FIG. 15, a representative mass spectrum of human Transthyretin obtained as outlined above is illustrated. Note the ability of the mass spectrometer to differentiate the truncated and isoforms of the protein.

### Example 4

### Analyte Capture, Electrochemical Cleavage and Analyte Focusing

Aliquots of sixteen human serum samples (25 μL) were added to 1.5 mL microcentrifuge tubes, diluted 1:1 with PBS Buffer, mixed by vortexing and centrifuged at 6000 rpm for 3 min to remove particulates. Two protein G/anti-Human Transthyretin biochips, prepared as described above, were placed as a pair in a 500 mL polycarbonate Nalgen® jar on top of damp sponge. Aliquots of supernatant (10 μL) were applied to each of 4 sites per biochip (64 sites total). The top was placed on the jar and the biochips incubated for 90 min at room temperature. Aliquots were shaken from the surfaces and the biochips individually washed once with 25 mL of PBS Buffer, pH 7.2 with 0.5% Triton X-100 for 30 min and twice with 25 mL of PBS Buffer, pH 7.2 for 15 min on an end-over-end shaker. Finally, the biochip surfaces were rinsed with 18 MΩ water from a wash bottle and blotted dry with Kimwipes®.

Ten microliter (10 μL) aliquots of 0.1 M perchloric acid in N,N-dimethylformamide electrolyte solution were added to each of 64 sites per biochip. A counter-electrode comprised of an array (8×8) of 64 platinum-electroplated pins arranged on 5.080 mm centers corresponding to the sites patterned on the surface of each biochip was positioned such that each pin contacted one aliquot of electrolyte solution on the surface of the biochip. The surface of the biochip and counter-electrode were connected to a BAS Epsilon potentiostat and a potential of +1450 mV applied to the surface of the biochip for 120 sec. Finally, the counter-electrode was removed and the aliquots of electrolyte solution evaporated.

Sinapinic acid matrix stock solution comprised of 1.0 mg/ml sinapinic acid in acetonitrile, ethanol and 5 mM ammonium citrate in 0.1% TFA (84:13:3) was prepared as follows: (1) 50 mM ammonium citrate in 0.1% TFA was prepared by dissolving 11.3 mg of ammonium citrate (dibasic) in 1.0 mL of 0.1% TFA in a 1.5 mL microcentrifuge tube by vortexing; (2) 5 mM ammonium citrate was prepared by adding 100 μL of 50 mM ammonium citrate in 0.1% TFA (step #1) to 900 μL of 0.1% TFA in a 1.5 ml micro-centrifuge tube and vortexing; (3) Solvent stock solution was prepared by adding 1000 μL of acetonitrile, 155 μL of ethanol and 36 μL of 5 mM ammonium citrate in 0.1% TFA (step #2) to a 1.5 ml microcentrifuge tube and vortexing; Sinapinic acid (10 mg/mL) in solvent stock solution was prepared by dissolving 5.0 mg of sinapinic acid in 500 μL of solvent stock solution (step #3) in a 1.5 mL microcentrifuge tube by vortexing; and (4) Sinapinic acid matrix stock solution was prepared by adding 35 μL of sinapinic acid in solvent stock solution (step #4) to 315 μL of solvent stock solution (step #3) in a 1.5 mL micro-centrifuge tube and vortexing.

Two microliter (2.0 μL) aliquots of sinapinic acid matrix stock solution prepared as described immediately above were added to each of 64 sites per biochip. As the aliquots evaporated a small droplet was localized above the area corresponding to the first affinity capture zone. Finally, sinapinic acid matrix crystals (white needles) were deposited above the area corresponding to the first affinity capture zone.

Each biochip was placed in stainless steel carrier, inserted into a Voyager DE MALDI-TOF-MS, and the instrument calibrated with respect to Cytochome c (external standard) in the positive ion-linear mode. Each sample site was subsequently analyzed by averaging the mass spectra corresponding to 20 laser shots at each of 20 locations (400 total mass spectra) associated with a spiral pattern emanating from the center of the sample site.

### Example 5

### MALDI-TOF-MS with Analyte Focusing

A series of protein G/anti-Human Transthyretin biochips were fabricated as described in Example 1 above, however, the final UV-photopatterning step was omitted during fabrication of half of the biochips. The resulting biochips, having either both first and second affinity capture zones or only second affinity capture zones, designated as focusing and non-focusing, respectively, were further modified as described in Example 3 above.

Aliquots of three human serum samples (25 μL) were added to 1.5 mL microcentrifuge tubes, diluted 1:1 with PBS Buffer, mixed by vortexing and centrifuged at 6000 rpm for 3 min to remove particulates. Two protein G/anti-Human Transthyretin biochips, focusing and non-focusing, were placed as a pair in a 500 mL polycarbonate Nalgene jar on top of damp sponge. Aliquots of different volumes of supernatant (10.0 μL, 5.0 μL and 2.5 μL) were applied to each of 4 sites per biochip (12 sites per biochip total). The top was placed on the jar and the biochips incubated for 90 min at room temperature. Aliquots were shaken from the surfaces and the biochips individually washed once with 25 mL of PBS Buffer, pH 7.2 with 0.5% Triton X-100 for 30 min and twice with 25 mL of PBS Buffer, pH 7.2 for 15 min on an end-over-end shaker. Finally, the biochip surfaces were rinsed with 18 MΩ water from a wash bottle and blotted dry with Kimwipes®.

Ten microliter (10 μL) aliquots of 0.1 M perchloric acid in N,N-dimethylformamide electrolyte solution were added to each of 12 sites per biochip. A counter-electrode comprised of an array (8×8) of 64 platinum-electroplated pins arranged on 5.080 mm centers corresponding to the sites patterned on the surface of each biochip was positioned such that each pin contacted one aliquot of electrolyte solution on the surface of the biochip. The surface of the biochip and counter-electrode were connected to a BAS Epsilon potentiostat and a potential of +1450 mV applied to the surface of the biochip for 120 sec. Finally, the counter-electrode was removed and the aliquots of electrolyte solution evaporated.

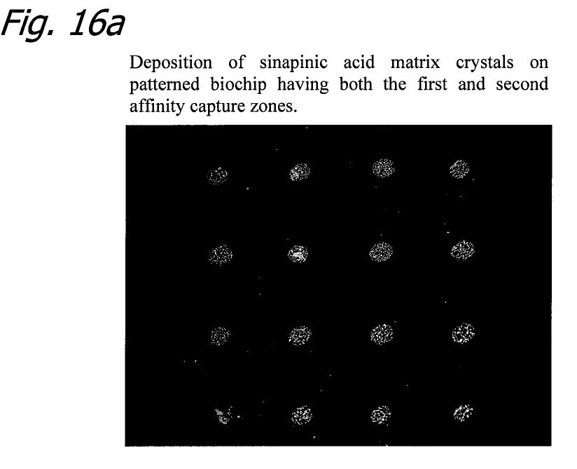

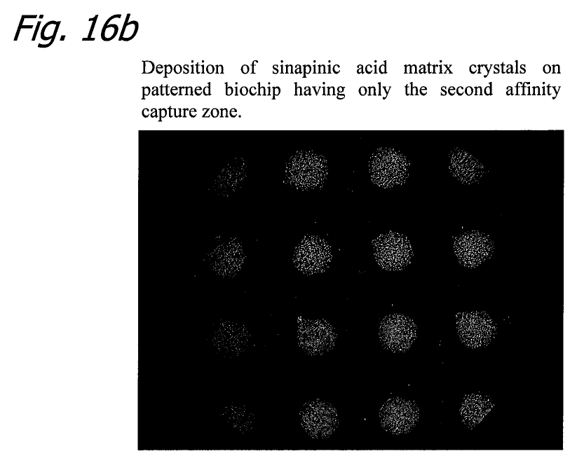

Two microliter (2.0 μL) aliquots of sinapinic acid matrix stock solution, prepared as described in Example 4 above, were added to each of 12 sites per biochip. As the aliquots evaporated a small droplet was localized above the area corresponding to the first affinity capture zone on the focusing biochip, but no localization of the evaporating droplet was observed for the non-focusing biochip. Finally, sinapinic acid matrix crystals were deposited above the area corresponding to the first affinity capture zone on the focusing biochip (see FIG. 16a) and above the area corresponding to the second affinity capture zone on the non-focusing biochip (see FIG. 16b).

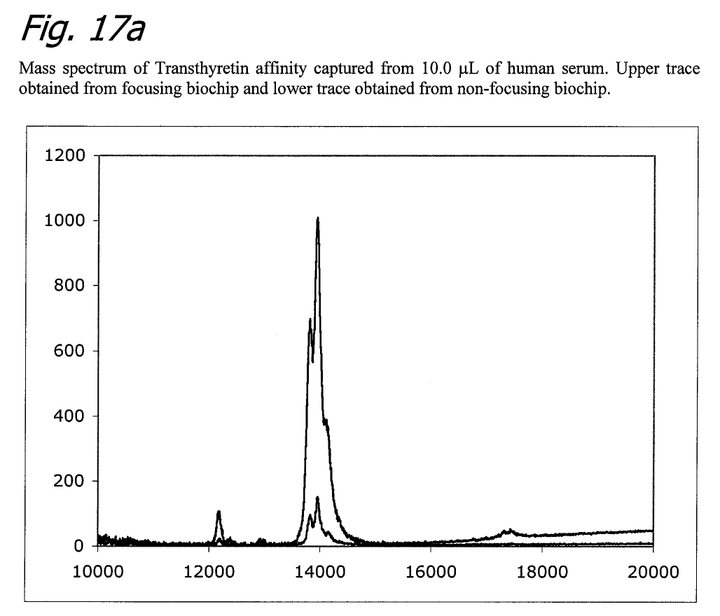

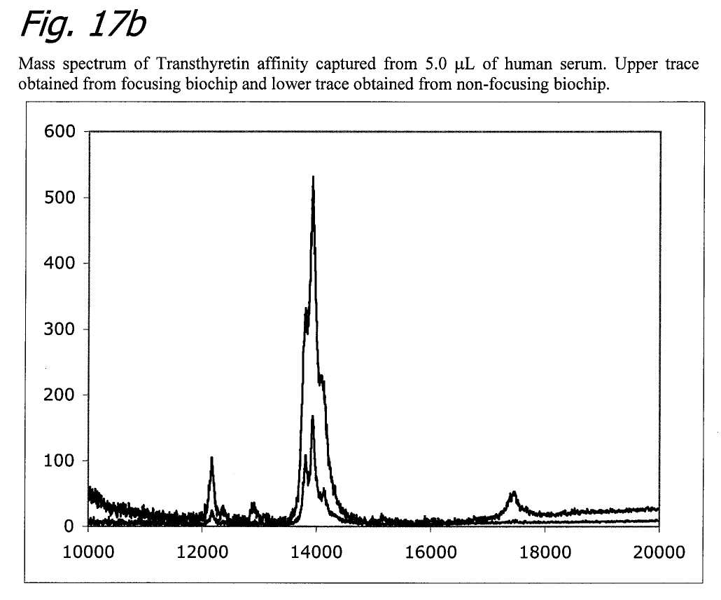

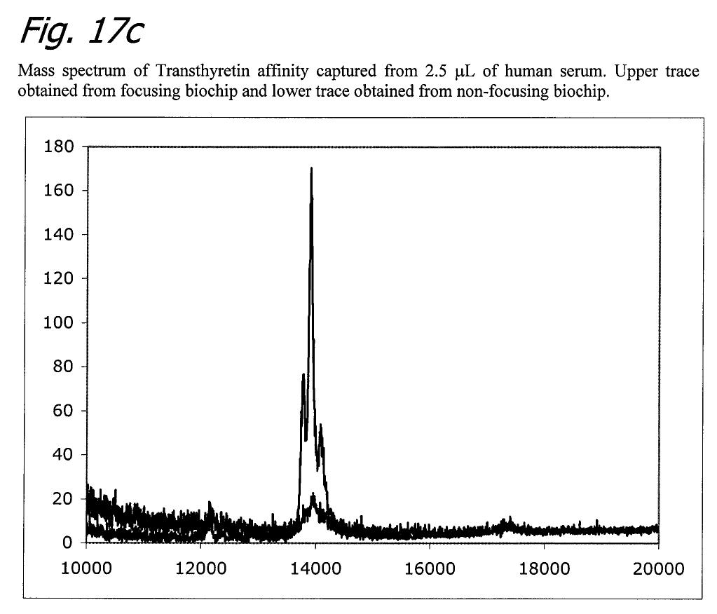

Each biochip was placed in stainless steel carrier, placed in the inlet of a Voyager DE MALDI-TOF-MS, the instrument evacuated and then calibrated with respect to Cytochome c (external standard) in the positive ion-linear mode. Each sample site was subsequently analyzed by averaging the mass spectra corresponding to 20 laser shots at each of 20 locations (400 total mass spectra) associated with a spiral pattern emanating from the center of the sample site. For focusing biochips, the spiral pattern had a diameter of 0.6 mm, and for non-focusing biochips the spiral pattern had a diameter of 3.0 mm. With reference to FIGS. 17a through 17c, the mass spectra corresponding to human serum sample volumes of 10.0, 5.0 and 2.5 μL, respectively, are illustrated.

### Example 6

A precursor to the alkanethiols of the present invention having a pendant electrochemically cleavable moiety, 4-(2-(2-(2-(11-mercaptoundecyloxy)-ethoxy)ethoxy)ethoxy)phenol (IV), was prepared as outlined in FIG. 18 (Synthetic Scheme 1).

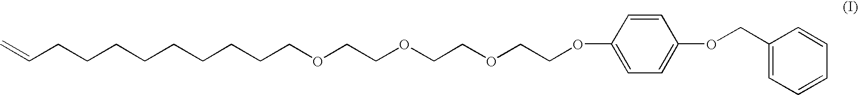

Methanesulfonic acid (2.55 g), 2-{2-[2-(undec-10-enyloxy)ethoxy]ethoxy}ethyl ester (6.7 mmol), 4-benzyloxyphenol (10.0 mmol, 2.0 g) and anhydrous potassium carbonate (20.0 mmol, 2.78 g) in dry acetonitrile (40 mL) were heated under reflux under an atmosphere of nitrogen gas for 20 hours. The product was cooled to room temperature, filtered and the solvent evaporated under vacuum. Finally, the residue was purified by silica gel column chromatography (petroleum ether/acetone, 8:1; then 6:1) to afford 2.9 g (89% yield) of 1-(benzyloxy)-4-(2-(2-(2-(undec-10-enyloxy)ethoxy)-ethoxy)ethoxy)benzene (I) as colorless oil. ¹H NMR (200 MHz, CDCl₃): δ 1.20-1.50 (m, CH₂, 12H), 1.50-1.70 (m, CH₂, 2H), 1.95-2.15 (m, CH₂—CH═CH₂, 2H), 3.46 (t, J=6.8 Hz, —CH₂—O, 2H), 3.52-3.78 (m, O—CH₂—CH₂—O, 8H), 3.80-3.90 (m, O—CH₂, 2H), 4.04-4.14 (m, O—CH₂, 2H), 4.86-5.08 (m, CH₂═CH—, 2H), 5.02 (s, CH₂-Ph, 2H), 5.70-5.64 (m, CH₂═CH—, 1H), 6.80-6.95 (m, O—C₆H₄—O, 4H), 7.28-7.48 (m, -Ph, 5H).

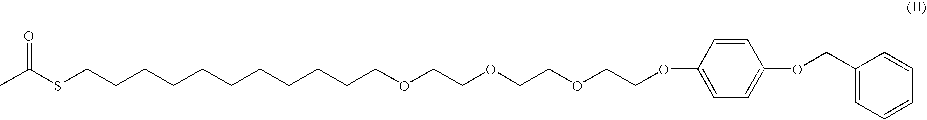

A solution of compound (I) (5.98 mmol, 2.9 g), thiolacetic acid (23.9 mmol, 1.8 g) and AIBN (0.3 mmol, 51 mg) in dry dichloromethane (50 mL) was irradiated in a photo-chemical reactor for 12 hours under an atmosphere of nitrogen gas. The solvent was evaporated under vacuum and the product purified by silica gel column chromatography (petroleum ether/acetone 6:1) to afford 2.29 g (70& yield) of S-11-(2-(2-(2-(4-(benzyl-oxy)phenoxy)ethoxy)ethoxy)ethoxy)undecyl ethanethioate (II) as a white solid (m.p. 55-57° C.). ¹H NMR (200 MHz, CDCl₃): δ 1.20-1.45 (m, CH₂, 14H), 1.45-1.68 (m, CH₂, 4H), 2.32 (s, CH₃COS, 3H), 2.87 (t, J=7.2 Hz, AcS—CH₂—, 2H), 3.45 (t, J=6.8 Hz, —CH₂—O, 2H), 3.52-3.78 (m, O—CH₂—CH₂—O, 8H), 3.80-3.88 (m, O—CH₂, 2H), 4.04-4.13 (m, O—CH₂, 2H), 5.02 (s, CH₂-Ph, 2H), 6.80-6.95 (m, O—C₆H₄—O, 4H), 7.28-7.48 (m, -Ph, 5H).

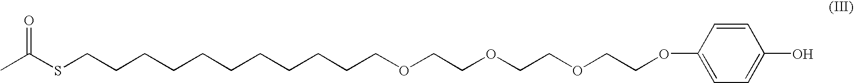

A solution of compound (II) (4.19 mmol, 2.29 g), 1,3-propanedithiol (16.8 mmol, 1.8 mL) and borontrifluoride etherate (16.8 mmol, 2.1 mL) in dry dichloromethane (50 mL) was stirred under an atmosphere of nitrogen gas for 4 hours. The solution was concentrated, dissolved in ethyl ether (50 mL), washed with water (3×20 mL), dried over anhydrous MgSO₄ and the solvent evaporated under vacuum. Finally, the residue was purified by silica gel column chromatography (petroleum ether/acetone 6:1; then 5:1) to afford 1.72 g (90% yield) of S-11-(2-(2-(2-(4-hydroxyphenoxy)ethoxy)ethoxy)-ethoxy)undecyl ethanethioate (III) as pale yellow oil. ¹H NMR (500 MHz, CDCl₃): δ 1.20-1.40 (m, CH₂, 14H), 1.51-1.62 (m, CH₂, 4H), 2.34 (s, CH₃COS, 3H), 2.87 (t, J=7.4 Hz, AcS—CH₂—, 2H), 3.45 (t, J=6.8 Hz, —CH₂—O, 2H), 3.55-3.78 (m, O—CH₂—CH₂—O, 8H), 3.80-3.88 (m, O—CH₂, 2H), 4.02-4.10 (m, O—CH₂, 2H), 6.72-6.82 (m, O—C₆H₄—O, 4H).

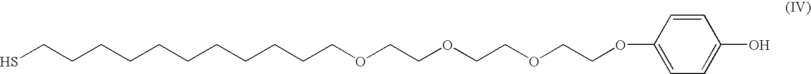

To a stirred solution of compound (III) (3.77 mmol, 1.72 g) in methanol (40 mL) was added hydrazine monoacetate (26.4 mmol, 2.43 g). After 18 hours, the solvent was evaporated, the residue dissolved in chloroform (50 mL) and washed with water (2×25 mL). The organic phase was dried over anhydrous MgSO₄ and the solvent evaporated under vacuum. Finally, the product was purified by silica gel column chromatography (petroleum ether/acetone: 6:1; and 5:1) to afford 1.09 g (70% yield) of 4-(2-(2-(2-(11-mercaptoundecyloxy)ethoxy)ethoxy)ethoxy)phenol (IV). ¹H NMR (500 MHz, CDCl₃): δ 1.20-1.40 (m, CH₂, 14H), 1.51-1.62 (m, CH₂, 4H), 2.54 (q, J=7.4 Hz, S—CH₂—, 2H), 3.45 (t, J=6.8 Hz, —CH₂—O, 2H), 3.55-3.78 (m, O—CH₂—CH₂—O, 8H), 3.80-3.90 (m, O—CH₂, 2H), 4.00-4.10 (m, O—CH₂, 2H), 6.70-6.85 (m, O—C₆H₄—O, 4H).

### Example 7

Eight gold-on-glass substrates measuring 44 mm² sputter coated with 250 Å of Inconel 600 and 5000 Å of gold (Erie Scientific, Portsmouth, N.H.) were placed in a Teflon carrier and submerged into a 250 mL beaker containing 150 mL of freshly prepared Piranha solution for 5 min with continuous stirring. The Teflon carrier was immediately transferred into a 250 mL beaker containing 150 mL of ethanol and stirred continuously for 15 min. The Teflon carrier was next transferred into a 250 mL beaker containing 150 mL of 18 MΩ water and stirred continuously for 15 min. Finally, the Teflon carrier was transferred into a 250 mL beaker containing 150 mL of ethanol and stirred continuously for a further 15 min.

Eight activated substrates in the Teflon carrier were submerged into a 250 mL beaker containing 150 mL of 0.01 mM 4-(2-(2-(2-(11-mercaptoundecyloxy)ethoxy)-ethoxy)ethoxy)phenol (IV) and 0.04 mM 2-(2-(2-(11-mercaptoundecyloxy)ethoxy)-ethoxy)ethanol in ethanol for 18 hours with continuous stirring. The resulting biochips in the Teflon carrier were transferred into a 250 mL beaker containing 150 mL of ethanol and stirred continuously for 15 min, and then individually transferred to a Spin Processor (Laurell Technologies, North Wales, Pa.) and washed with a stream of ethanol for 1 min at 2500 rpm. Finally, the biochips were allowed to dry on the Spin Processor for 30 sec at 2500 rpm.

One of the eight biochips prepared as described above was placed in a flat electrochemical cell with a window measuring 1 cm². The cell was filled with 250 mL of 0.1 M perchloric acid/acetonitrile 4:1 and fitted with a platinum screen counterelectrode and Ag/AgCl/KCl reference electrode. Dry nitrogen gas was bubbled through the buffer for 10 min to remove dissolved oxygen. The cell was connected to an Epsilon potentiostat (BASi, West Lafayette, Ind.) and three cyclic voltammograms recorded at 100 mV/sec between 0 mV and +1000 mV. A peak corresponding to the oxidation of the terminal phenol moiety to a para quinone was detected at +940 mV. The corresponding reduction peak was not detected, presumably due to diffusion of the liberated quinone moiety away from the surface of the working electrode.

### Example 8

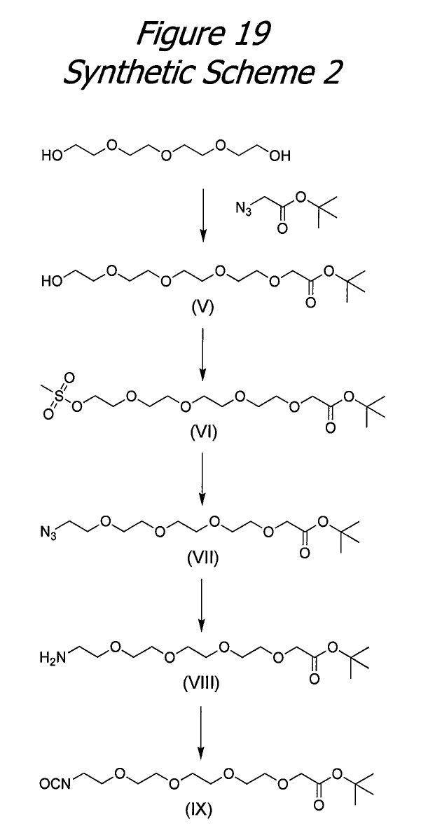

Two key synthetic intermediates required for subsequent syntheses, (2-{2-[2-(2-aminoethoxy)ethoxy]ethoxy}ethoxy)acetic acid tert-butyl ester (VIII) and (2-{2-[2-(2-isocyanatoethoxy)ethoxy]ethoxy}ethoxy)acetic acid tert-butyl ester (IX), were prepared as outlined in FIG. 19 (Synthetic Scheme 2).

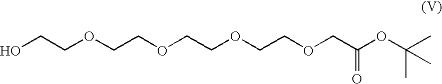

Three drops of freshly distilled borontrifluoride etherate were added to a stirred mixture of tetraethylene glycol (28 mmol, 5.44 g) and tert-butyl diazoacetate (14 mmol, 2.0 g) in dry dichloromethane (35 mL) under nitrogen gas. After 15 min, 3 further drops of borontrifluoride etherate were added and the resulting mixture was stirred for 1 hour. The solvent was removed under vacuum and the product purified by silica gel column chromatography (two columns: chloroform/methanol 2:1; then neat ethyl acetate) to afford 1.5 g (35% yield) of (2-{2-[2-(2-hydroxyethoxy)ethoxy]ethoxy}ethoxy)acetic acid tert-butyl ester (V) as colorless oil. ¹H NMR (200 MHz, CDCl₃): δ 1.46 (s, tBu, 9H), 2.86 (brs, OH, 1H), 3.55-3.80 (m, O—CH₂—CH₂—O, 16H), 4.01 (s, —O—CH₂COOtBu, 2H).

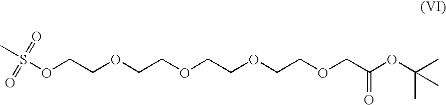

A stirred solution of compound (V) (4.86 mmol, 1.5 g) and triethylamine (12.2 mmol, 1.23 g) in dry dichloromethane (40 mL) was cooled to 0° C. under nitrogen gas and methanesulfonyl chloride (9.72 mmol, 1.11 g) was added. The reaction mixture was stirred for 16 hours, washed with water (20 mL), dried over anhydrous magnesium sulfate and the solvent evaporated under vacuum. Finally, the residue was purified by silica gel column chromatography (two columns: chloroform/methanol 40:1; then petroleum ether/ethyl acetate 1:1) to afford 1.2 g (64% yield) of (2-{2-[2-(2-mesyloxyethoxy)ethoxy]ethoxy}ethoxy)acetic acid tert-butyl ester (VI) as colorless oil. ¹H NMR (200 MHz, CDCl₃): δ 1.45 (s, tBu, 9H), 3.06 (s, CH₃SO₃—, 3H), 3.50-3.80 (m, O—CH₂—CH₂—O, 14H), 3.99 (s, —O—CH₂COOtBu, 2H), 4.30-4.40 (m, CH₃SO₂—O—CH₂—CH₂—O, 2H).

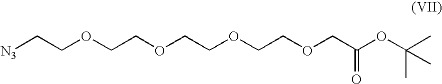

A solution of compound (VI) (3.1 mmol, 1.2 g) and sodium azide (6.2 mmol, 400 mg) in dry dimethylformamide (30 mL) under nitrogen gas was stirred for 19 hours. The reaction mixture was concentrated under vacuum, dissolved in dichloromethane (50 mL), washed with water (20 mL) and dried over anhydrous magnesium sulfate. The solvent was evaporated under vacuum and the residue purified by silica gel column chromatography (chloroform/methanol 40:1) to afford 0.83 g (80% yield) of (2-{2-[2-(2-azidoethoxy)ethoxy]ethoxy}ethoxy)acetic acid tert-butyl ester (VII) as colorless oil. ¹H NMR (200 MHz, CDCl₃): δ 1.46 (s, tBu, 9H), 3.40 (t, J=5.3 Hz, N₃—CH₂—CH₂—O—, 2H), 3.60-3.70 (m, O—CH₂—CH₂—O, 14H), 4.00 (s, —O—CH₂COOtBu, 2H).

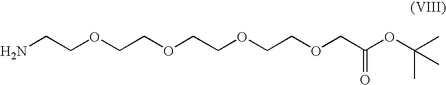

A solution of compound (VII) (1.5 mmol, 0.5 g) and 400 mg of 10% Pd/C catalyst in ethanol/chloroform 10:1 (25 mL) was stirred for 18 hours under a hydrogen atmosphere. The catalyst was removed by filtration over Celite and the filtrate concentrated under reduced pressure. The crude product was co-evaporated with dry chloroform (2×50 mL) to afford 0.46 g (100% yield) of (2-{2-[2-(2-aminoethoxy)-ethoxy]ethoxy}ethoxy)acetic acid tert-butyl ester (VIII) as colorless oil.

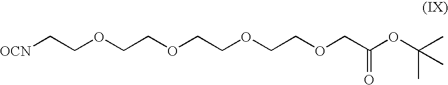

A solution of compound (VIII) (0.325 mmol, 100 mg) and phosgene (0.65 mmol, 20% in toluene) in dry dichloromethane (5 mL) was stirred at 0° C. and triethylamine (33 mg) was added. The reaction mixture was stirred under an atmosphere of nitrogen gas for 20 hours. Then excess of phosgene and toluene was evaporated under reduced pressure to afford (2-{2-[2-(2-isocyanatoethoxy)ethoxy]ethoxy}ethoxy)acetic acid tert-butyl ester (IX).

### Example 9

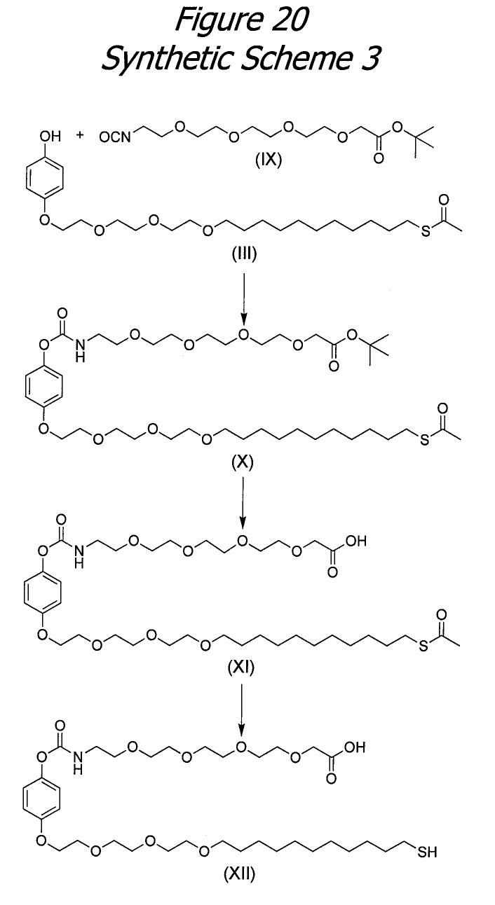

An alkanethiol of the present invention having a pendant reactive moiety useful for immobilization of an affinity capture agent, 1-(4-(2-(2-(2-(11-mercaptoundecyl-oxy)-ethoxy)ethoxy)ethoxy)phenoxy)-1-oxo-5,8,11,14-tetraoxa-2-azahexadecan-16-oic acid (XII), was prepared in three steps as outlined in FIG. 20 (Synthetic Scheme 3).

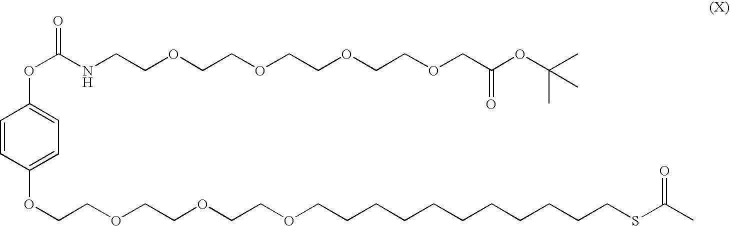

A solution of compound (III) (0.328 mmol, 150 mg) and diisopropylethylamine (0.325 mmol, 42 mg) in dry dichloromethane (1 mL) was added to the solution of compound (IX) (0.325 mmol, 108 mg) in dry dichloromethane (10 mL). The reaction mixture was stirred under an atmosphere of nitrogen gas for 20 hours. The solvent was evaporated under vacuum and the product purified by silica gel column chromatography (petroleum ether/acetone: 6:1 and 2:1) to afford 45 mg (17% yield) of tert-butyl 1-oxo-1-(4-(22-oxo-3,6,9-trioxa-21-thiatricosyloxy)phenoxy)-5,8,11,14-tetraoxa-2-azahexadecan-16-oate (X). ¹H NMR (500 MHz, CDCl₃): δ 1.20-1.40 (m, CH₂, 14H), 1.48 (s, t-Bu, 9H), 1.51-1.62 (m, CH₂, 4H), 2.33 (s, CH₃COS, 3H), 2.87 (t, J=7.3 Hz, AcS—CH₂—, 2H), 3.42-3.51 (m, —CH₂—O, CONH—CH₂—, 4H), 3.55-3.78 (m, O—CH₂—CH₂—O, 22H), 3.82-3.90 (m, O—CH₂, 2H), 4.03 (s, —O—CH₂COOtBu, 2H), 4.10-4.18 (m, O—CH₂, 2H), 5.76 (brs, NH, 1H), 6.88 (d, J=9.0 Hz, O—C₆H₄—O, 2H), 7.03 (d, J=9.0 Hz, O—C₆H₄—O, 2H).

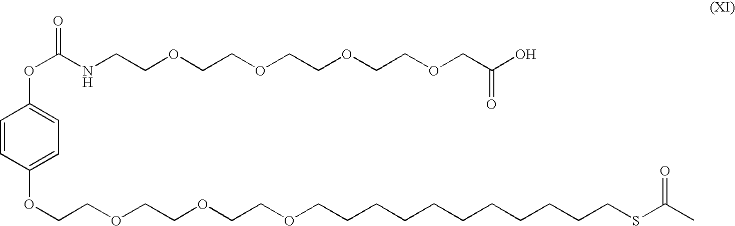

To a cooled solution on ice of compound (X) (0.056 mmol, 45 mg) in dichloromethane (1.5 mL), trifluoroacetic acid (0.5 mL) was added and reaction mixture was stirred for 4.5 hours at 0° C. under an atmosphere of nitrogen gas. The solvent was evaporated under vacuum and the residue dissolved in dry toluene (10 mL). The toluene was evaporated under vacuum and the residue again dissolved in toluene (10 mL). Finally, the toluene was evaporated under vacuum to afford 40 mg (95% yield) of 1-oxo-1-(4-(22-oxo-3,6,9-trioxa-21-thiatricosyloxy)phenoxy)-5,8,11,14-tetra-oxa-2-azahexadecan-16-oic acid (XX) as yellowish oil. ¹H NMR (200 MHz, CDCl₃): δ 1.10-1.45 (m, CH₂, 14H), 1.45-1.70 (m, CH₂, 4H), 2.33 (s, CH₃COS, 3H), 2.87 (t, J=7.3 Hz, AcS—CH₂—, 2H), 3.20-3.95 (m, —CH₂—O, CONH—CH₂—, O—CH₂—CH₂—O, 28H), 4.00-4.20 (m, O—CH₂, 2H), 4.03 (s, —O—CH₂COOtBu, 2H), 5.80 (brs, NH, 1H), 6.88 (d, J=9.0 Hz, O—C₆H₄—O, 2H), 7.03 (d, J=9.0 Hz, O—C₆H₄—O, 2H).

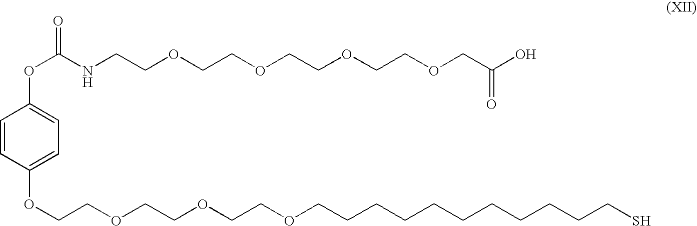

To a solution of compound (XI) (0.027 mmol, 20 mg) in dry tetrahydrofuran (4 mL), hydrazine monoacetate (0.111 mmol, 10 mg) in dry dimethylformamide (0.1 mL) was added. After 20 hours, the solvent was evaporated under vacuum. The residue was dissolved in chloroform (50 mL), washed with water (3×20 mL), dried over anhydrous MgSO₄ and evaporated under vacuum to afford 1-(4-(2-(2-(2-(11-mercaptoundecyl-oxy)ethoxy)ethoxy)ethoxy)phenoxy)-1-oxo-5,8,11,14-tetraoxa-2-azahexadecan-16-oic acid (XII). ¹H NMR spectrum of crude product revealed the presence of disulfide and thiol (major product). ¹H NMR (500 MHz, CDCl₃): δ 1.20-1.40 (m, CH₂, 14H), 1.52-1.65 (m, CH₂, 4H), 2.52 (q, J=7.3 Hz, HS—CH₂—, 2H), 3.42-3.51 (m, —CH₂—O, CONH—CH₂—, 4H), 3.55-3.78 (m, O—CH₂—CH₂−O, 22H), 3.82-3.90 (m, O—CH₂, 2H), 4.10-4.18 (m, O—CH₂, 2H), 4.14 (s, —O—CH₂COOH, 2H), 5.86 (brs, NH, 1H), 6.88 (d, J=9.0 Hz, O—C₆H₄—O, 2H), 7.03 (d, J=9.0 Hz, O—C₆H₄—O, 2H).

### Example 10

A self-assembled monolayer of the present invention (shown in FIG. 21a), comprised of approx. 10% 1-(4-(2-(2-(2-(11-mercaptoundecyloxy)ethoxy)ethoxy)ethoxy)-phenoxy)-1-oxo-5,8,11,14-tetraoxa-2-azahexadecan-16-oic acid (XII) and approx. 90% 2-(2-(2-(11-mercaptoundecyloxy)ethoxy)ethoxy)ethanol, was prepared as described below.

Eight gold-on-glass substrates measuring 44 mm×44 mm, sputter coated with Inconel® (50 Å) and gold (5000 Å) and silk-screened with hydrophobic ink to provide an array of 3.0 mm O.D. exposed gold sites on 5.080 mm centers (Erie Scientific, Portsmouth, N.H.) were placed in the chamber of a PSD-UV UV/ozone cleaning system (Novascan, Ames, Iowa) irradiated for 15 min and then allowed to incubate in the presence of ozone for a further 15 min. The substrates were transferred to a Teflon carrier and washed with ethanol for 15 min with continuous stirring, and then removed from the carrier and dried under a stream of dry nitrogen gas. The substrates were again placed in the chamber of the PSD-UV cleaning system and irradiated for 15 min and then allowed to incubate in the presence of ozone for a further 15 min. Finally, the substrates were again transferred to a Teflon carrier and washed with ethanol with continuous stirring until modified as described below.

UV/Ozone activated substrates in a Teflon carrier were submerged into a 250 mL beaker containing 150 mL of 0.1 mM (XII) in ethanol for 60 min with continuous stirring. After 60 min, the carrier was removed and 250 μL of a stock solution of 2-(2-(2-(11-mercaptoundecyloxy)ethoxy)ethoxy)ethanol at 0.2 mg/μL was added and the solution stirred for 3 min. Finally, the Teflon carrier was returned to the solution and the surface modification reaction was stirred continuously for approximately 18 hours. The resulting biochips in the Teflon carrier were transferred into a 250 mL beaker containing 150 mL of ethanol and stirred continuously for 15 min and then individually transferred to a Spin Processor (Laurell Technologies, North Wales, Pa.) and washed with a stream of ethanol for 1 min at 2500 rpm. Finally, the biochips were allowed to dry on the Spin Processor for 30 sec at 2500 rpm.

One biochip prepared as described above was placed in a flat electrochemical cell with window measuring 1 cm². The cell was filled with 250 mL of 0.1 M perchloric acid and fitted with a platinum screen counterelectrode and Ag/AgCl/KCl reference electrode. Dry nitrogen gas was bubbled through the buffer for 10 min to remove dissolved oxygen. The cell was connected to a BAS Epsilon potentiostat and three cyclic voltammograms recorded at 100 mV/sec between 0 mV and +950 mV. A peak corresponding to the oxidation of the terminal electrochemically active moiety to a para quinone was detected at +810 mV (see FIG. 21b, Cycle #1). The corresponding reduction peak was not detected, due to diffusion of the liberated quinone moiety away from the surface of the working electrode. Cyclic voltammograms corresponding to Cycles #2 and #3 indicate the absence of an electrochemically active moiety and demonstrate the stability of the residual self-assembled monolayer.

### Example 11

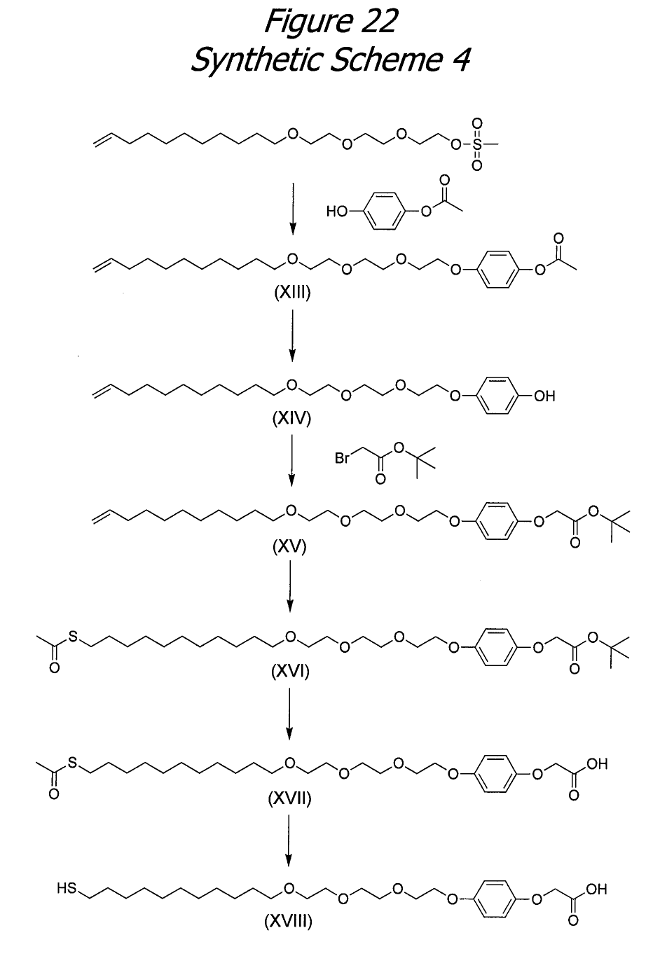

An alkanethiol having a pendant reactive moiety useful as an intermediary for the preparation of self-assembled monolayers of the present invention, 2-(4-(2-(2-(2-(11-mercaptoundecyloxy)ethoxy)ethoxy)ethoxy)phenoxy)acetic acid (XVIII), was prepared in six steps as outlined in FIG. 22 (Synthetic Scheme 4).

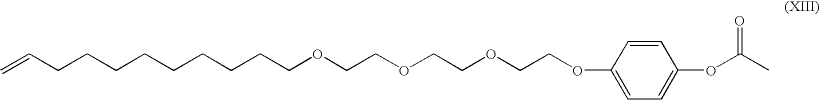

A mixture of methanesulfonic acid (26.5 mmol, 2.55 g) and 2-{2-[2-(undec-10-enyloxy)ethoxy]ethoxy}ethyl ester (6.7 mmol), 4-acetyloxyphenol (10.0 mmol, 1.52 g) and anhydrous K₂CO₃ (20.0 mmol, 2.78 g) in dry acetonitrile (40 mL) was heated under reflux under an atmosphere of nitrogen for 20 hours. The reaction mixture was cooled to room temperature, filtered, and concentrated under vacuum. The residue was purified by silica gel column chromatography (petroleum ether/acetone: 8:1 and 6:1) to afford 1.76 g (60% yield) of 4-(2-(2-(2-(undec-10-enyloxy)ethoxy)ethoxy)ethoxy)-phenyl acetate (XIII) as colorless oil. ¹H NMR (200 MHz, CDCl₃): δ 1.20-1.50 (m, CH₂, 12H), 1.50-1.70 (m, CH₂, 2H), 1.95-2.15 (m, CH₂—CH═CH₂, 2H), 2.35 (s, CH₃CO, 3H), 3.47 (t, J=6.8 Hz, —CH₂—O, 2H), 3.50-3.76 (m, O—CH₂—CH₂—O, 8H), 3.80-3.90 (m, O—CH₂, 2H), 4.04-4.14 (m, O—CH₂, 2H), 4.86-5.08 (m, CH₂═CH—, 2H), 5.70-5.64 (m, CH₂═CH—, 1H), 6.80-6.95 (m, O—C₆H₄—O, 4H).

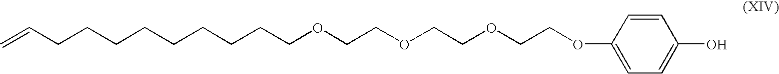

To a stirred solution of compound (XIII) (4.58 mmol, 2.00 g) in methanol (40 mL), hydrazine monoacetate (32 mmol, 2.94 g) is added. After 18 hours, the solvent was evaporated under vacuum, the residue dissolved in chloroform (50 mL) and washed with water (25 mL) and dried over anhydrous MgSO₄. The solvent was evaporated under vacuum and the product purified by silica gel column chromatography (petroleum ether/acetone 1:8) to afford 1.50 g (83% yield) of 4-(2-(2-(2-(undec-10-enyloxy)ethoxy)ethoxy)ethoxy)phenol (XIV). ¹H NMR (200 MHz, CDCl₃) δ=1.20-1.50 (m, CH₂, 12H), 1.50-1.70 (m, CH₂, 2H), 1.95-2.15 (m, CH₂—CH═CH₂, 2H), 3.45 (t, J=6.8 Hz, —CH₂—O, 2H), 3.52-3.78 (m, O—CH₂—CH₂—O, 8H), 3.75-3.85 (m, O—CH₂, 2H), 3.95-4.10 (m, O—CH₂, 2H), 4.86-5.08 (m, CH₂═CH—, 2H), 5.70-5.95 (m, CH₂═CH—, 1H), 6.74 (s, O—C₆H₄—O, 4H).

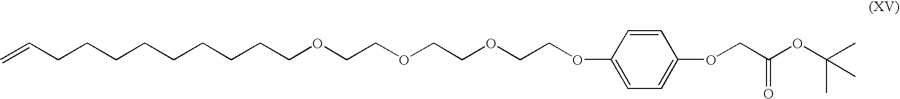

To a solution of compound (XIV) (5.50 mmol, 2.1 g) in dry tetrahydrofuran (50 mL), sodium hydride (6.60 mmol, 226 mg of 60% in mineral oil) was added followed by tert-butyl bromoacetate (5.51 mmol, 1.07 g), and the mixture was stirred under reflux for 20 hours under an atmosphere of nitrogen. The reaction mixture was evaporated under vacuum, the residue dissolved in ethyl acetate (100 mL) and washed with saturated ammonium chloride solution (50 mL). The aqueous phase was extracted with ethyl acetate (30 mL). The organic phases were dried over anhydrous MgSO₄ and evaporated under vacuum. Finally, the product was purified by silica gel column chromatography (dichloromethane; and dichloromethane/diethyl ether 20:1) to afford 2.20 g (79% yield) of tert-butyl 2-(4-(2-(2-(2-(undec-10-enyloxy)ethoxy)ethoxy)ethoxy)-phenoxy)acetate (XV). ¹H NMR (200 MHz, CDCl₃): δ 1.20-1.45 (m, CH₂, 12H), 1.49 (s, t-Bu, 9H) 1.50-1.70 (m, CH₂, 2H), 1.95-2.15 (m, CH₂—CH═CH₂, 2H), 3.45 (t, J=6.8 Hz, —CH₂—O, 2H), 3.52-3.78 (m, O—CH₂—CH₂—O, 8H), 3.80-3.88 (m, O—CH₂, 2H), 4.04-4.14 (m, O—CH₂, 2H), 4.46 (s, O—CH₂COOtBu, 2H), 4.80-5.05 (m, CH₂═CH—, 2H), 5.70-5.94 (m, CH₂═CH—, 1H), 6.84 (s, O—C₆H₄—O, 4H).

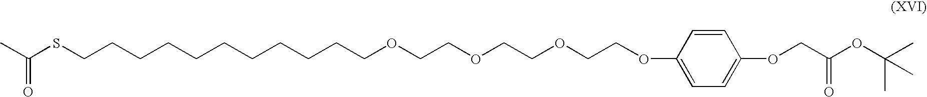

A solution of compound (XV) (5.41 mmol, 2.75 g), thioacetic acid (28.0 mmol) and AIBN (0.3 mmol, 51 mg) in methanol (75 mL) was irradiated in a photo-chemical reactor for 3 hours under an atmosphere of nitrogen. Then the solvent was evaporated under vacuum and the product purified by silica gel column chromatography (dichloromethane; dichloromethane/diethyl ether 40:1) to afford 2.24 g (71% yield) of tert-butyl 2-(4-(22-oxo-3,6,9-trioxa-21-thiatricosyloxy)phenoxy)acetate (XVI). ¹H NMR (200 MHz, CDCl₃): δ 1.20-1.45 (m, CH₂, 14H), 1.49 (s, t-Bu, 9H), 1.51-1.70 (m, CH₂, 4H), 2.33 (s, CH₃COS, 3H), 2.85 (t, J=7.3 Hz, AcS—CH₂—, 2H), 3.44 (t, J=6.8 Hz, —CH₂—O, 2H), 3.55-3.76 (m, O—CH₂—CH₂—O, 8H), 3.80-3.88 (m, O—CH₂, 2H), 4.05-4.15 (m, O—CH₂, 4H), 4.45 (s, —O—CH₂COOtBu, 2H), 6.83 (s, O—C₆H₄—O, 4H).

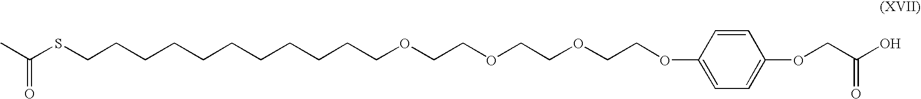

A solution of compound (XVI) (0.855 mmol, 500 mg) in dry dichloromethane (1 mL) and trifluoroacetic acid (4 mL) was stirred for 3 hours. The solvent was evaporated under vacuum and the residue dissolved in dry toluene (10 mL). The toluene was evaporated under vacuum and the residue again dissolved in toluene (10 mL). The toluene was evaporated under vacuum and the product purified by silica gel flash chromatography (dichloromethane/methanol: 20:1 and 10:1) to afford 400 mg (88% yield) of 2-(4-(22-oxo-3,6,9-trioxa-21-thiatricosyloxy)phenoxy)acetic acid (XVII). ¹H NMR (500 MHz, CDCl₃): δ 1.20-1.45 (m, CH₂, 14H), 1.52-1.65 (m, CH₂, 4H), 2.34 (s, CH₃COS, 3H), 2.87 (t, J=7.3 Hz, AcS—CH₂—, 2H), 3.46 (t, J=6.8 Hz, —CH₂—O, 2H), 3.58-3.78 (m, O—CH₂—CH₂—O, 8H), 3.82-3.90 (m, O—CH₂—CH₂—O, 2H), 4.08-4.15 (m, O—CH₂, 2H), 4.61 (s, —O—CH₂COO, 2H), 6.87 (s, O—C₆H₄—O, 4H).

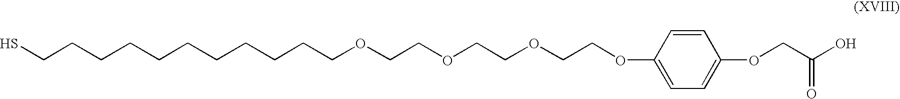

A solution of compound (XVII) (0.719 mmol, 380 mg) and hydrazine mono-acetate (7.19 mmol, 660 mg) in methanol (30 mL) was stirred for 18 hours. The solvent was evaporated under vacuum, the residue dissolved in chloroform (50 mL) and washed with water (25 mL). The organic phase was dried over anhydrous MgSO₄ and the solvent evaporated under vacuum. Finally, the product was purified by silica gel column chromatography (dichloromethane/methanol: 50:1; 20:1; and 10:1) to afford 280 mg (80% yield) of 2-(4-(2-(2-(2-(11-mercaptoundecyloxy)ethoxy)ethoxy)ethoxy)-phenoxy)acetic acid (XVIII). ¹H NMR (500 MHz, CDCl₃): δ 1.20-1.46 (m, CH₂, 14H), 1.51-1.65 (m, CH₂, 4H), 2.53 (q, J=7.3 Hz, HS—CH₂—, 2H), 3.46 (t, J=6.8 Hz, —CH₂—O, 2H), 3.58-3.78 (m, O—CH₂—CH₂—O, 8H), 3.82-3.90 (m, O—CH₂—CH₂—O, 2H), 4.08-4.15 (m, O—CH₂, 2H), 4.45 (s, —O—CH₂COO, 2H), 6.82 (s, O—C₆H₄—O, 4H).

### Example 12

A self-assembled monolayer of the present invention (shown in FIG. 23a), comprised of approx. 10% 1-(4-(2-(2-(2-(11-mercaptoundecyloxy)ethoxy)ethoxy)ethoxy)phenoxy)-2-oxo-6,9,12,15-tetraoxa-3-azaheptadecan-17-oic acid (XVIII) and approx. 90% 2-(2-(2-(11-mercapto-undecyloxy)ethoxy)ethoxy)ethanol, was prepared as described below.

Eight gold-on-glass substrates measuring 44 mm×44 mm, sputter coated with Inconel® (50 Å) and gold (5000 Å) and silk-screened with hydrophobic ink to provide an array of 3.0 mm O.D. exposed gold sites on 5.080 mm centers (Erie Scientific, Portsmouth, N.H.) were placed in the chamber of a PSD-UV UV/ozone cleaning system (Novascan, Ames, Iowa) irradiated for 15 min and then allowed to incubate in the presence of ozone for a further 15 min. The substrates were transferred to a Teflon carrier and washed with ethanol for 15 min with continuous stirring, and then removed from the carrier and dried under a stream of dry nitrogen gas. The substrates were again placed in the chamber of the PSD-UV cleaning system and irradiated for 15 min and then allowed to incubate in the presence of ozone for a further 15 min. Finally, the substrates were again transferred to a Teflon carrier and washed with ethanol with continuous stirring until modified as described below.

Eight UV/Ozone activated gold-on-glass substrates were prepared as described above and the gold surface modified by potential-assisted deposition as follows: Individual substrates were placed in a custom-fabricated Teflon electrochemical cell insert designed to position counter and reference electrodes in proximity to the substrate surface while minimizing the volume of solution required. The insert was fitted with a platinum wire counter electrode, Ag/AgCl/KCl reference electrode and a gold-plated alligator clip contacting the surface of the substrate and enabling the working electrode to be attached above the solution contained in the cell. The substrate and electrodes were connected to a BAS Epsilon potentiostat. The cell insert was immersed into a 100 mL beaker containing 40 mL of 0.05 mM Reagent (XVIII) and 0.45 mM 2-(2-(2-(11-mercaptoundecyloxy)ethoxy)ethoxy)ethanol in ethanol. A potential of +300 mV was applied to the surface of the substrate for 10 min. Over the time course of the deposition, the change in current (μA) vs. time (min) was recorded. The resulting biochip was then transferred to the Spin Processor (Laurell Technologies, North Wales, Pa.) and washed with a stream of ethanol for 1 min at 2500 rpm and then allowed to dry for 30 sec at 2500 rpm. Three additional substrates were modified as described above utilizing the same thiol solution and the remaining four substrates modified after preparing fresh thiol solution.

One biochip prepared by potential-assisted deposition as described above was placed in a flat electrochemical cell with a window measuring 1 cm². The cell was filled with 250 mL of 0.1 M perchloric acid in dry acetonitrile and fitted with a platinum screen counterelectrode and Ag/AgCl/KCl reference electrode. The cell was connected to a BAS Epsilon potentiostat and three cyclic voltammograms recorded at 100 mV/sec between +450 mV and +1450 mV. A peak corresponding to the oxidation of the terminal electrochemically active moiety to a para quinone was detected at approx. +1400 mV (see FIG. 23b, Cycle #1). The corresponding reduction peak was not detected, due to diffusion of the liberated quinone moiety away from the surface of the working electrode. Cyclic voltammograms corresponding to Cycles #2 and #3 indicate the absence of an electrochemically active moiety and demonstrate the stability of the residual self-assembled monolayer.

### Example 13

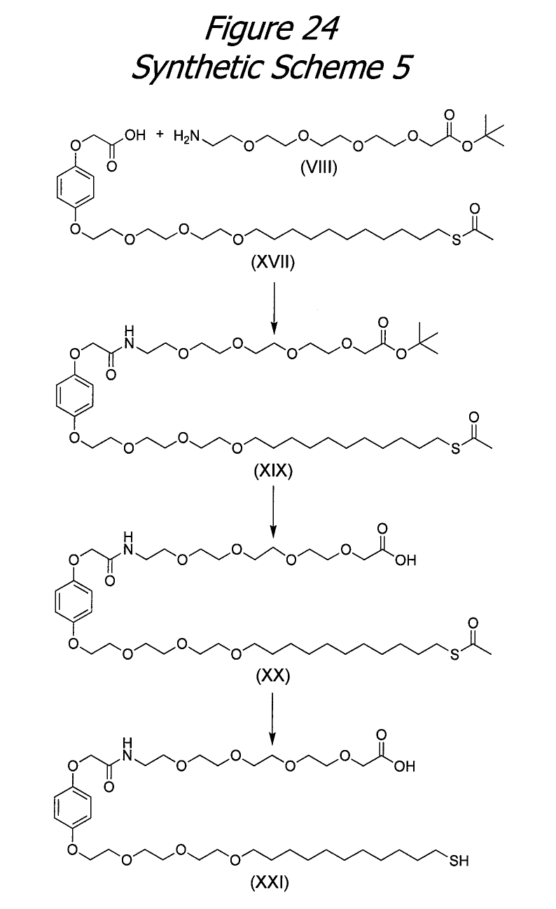

An alkanethiol of the present invention having a pendant reactive moiety useful for immobilization of an affinity capture agent, 1-(4-(2-(2-(2-(11-mercaptoundecyl-oxy)ethoxy)ethoxy)ethoxy)phenoxy)-2-oxo-6,9,12,15-tetraoxa-3-azaheptadecan-17-oic acid (XXI), was prepared in three steps as outlined in FIG. 24 (Synthetic Scheme 5).

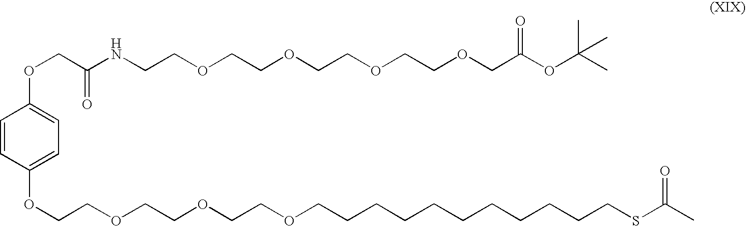

A solution of compound (XVII) (2.6 mmol, 1.4 g) and N-hydroxysuccinimide (3.9 mmol, 452 mg) in dry tetrahydrofuran (15 mL) was cooled on ice and dicyclo-hexylcarbodiimide (3.9 mmol, 804 mg) was added. The suspension was stirred at 0° C. under a nitrogen atmosphere for 3 hours. The precipitate was removed by filtration, and compound (VIII) (3.2 mmol, 1.0 g) and triethylamine (2.6 mmol, 0.36 mL) were added to the filtrate. The reaction was stirred at for 16 hours at room temperature. The solvent was evaporated under vacuum and the product purified by silica gel column chromatography (diethyl ether/acetone: 20:1; 10:1; and 5:1) to afford 1.3 g (61% yield) of tert-butyl 2-oxo-1-(4-(22-oxo-3,6,9-trioxa-21-thiatricosyloxy)phenoxy)-6,9,12,15-tetra-oxa-3-azaheptadecan-17-oate (XIX). ¹H NMR (200 MHz, CDCl₃): δ 1.20-1.40 (m, CH₂, 14H), 1.48 (s, t-Bu, 9H), 1.50-1.65 (m, CH₂, 4H), 2.33 (s, CH₃COS, 3H), 2.85 (t, J=7.3 Hz, AcS—CH₂—, 2H), 3.44 (t, J=6.7 Hz, O—CH₂—, 2H), 3.50-3.78 (m, O—CH₂—CH₂—O, NH—CH₂—CH₂—O, 24H), 3.80-3.90 (m, O—CH₂, 2H), 4.00 (s, —O—CH₂COOtBu, 2H), 4.05-4.15 (m, O—CH₂, 2H), 4.43 (s, Ar—O—CH₂CONH, 2H), 6.85 (s, O—C₆H₄—O, 4H), 7.05 (brs, NH, 1H).

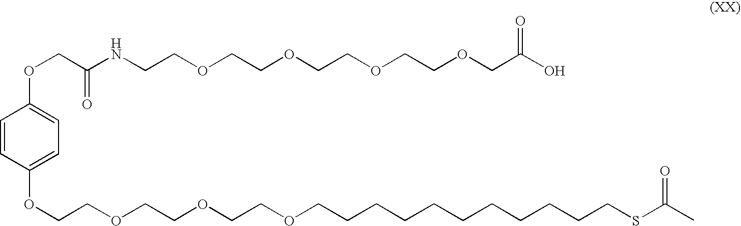

To a solution of compound (XIX) (0.1 mmol, 82 mg) in dichloromethane (1.5 mL), trifluoroacetic acid (0.5 mL) was added and reaction was stirred for 4.5 hours at 0° C. under an atmosphere of nitrogen gas. Then solvent was evaporated under vacuum and the residue dissolved in dry toluene (10 mL). The toluene was evaporated under vacuum and the product purified by silica gel column chromatography (chloroform/methanol: 30:1; 25:1; 20:1; and 10:1) to afford 68 mg (89% yield) of tert-butyl 2-oxo-1-(4-(22-oxo-3,6,9-trioxa-21-thiatricosyloxy)phenoxy)-6,9,12,15-tetraoxa-3-azaheptadecan-17-oate (XX) as yellowish oil. ¹H NMR (500 MHz, CDCl₃): δ 1.20-1.40 (m, CH₂, 14H), 1.52-1.62 (m, CH₂, 4H), 2.33 (s, CH₃COS, 3H), 2.87 (t, J=7.3 Hz, AcS—CH₂—, 2H), 3.46 (t, J=6.7 Hz, O—CH₂—, 2H), 3.54-3.77 (m, O—CH₂—CH₂—O, NH—CH₂—CH₂—O, 24H), 3.83-3.88 (m, O—CH₂, 2H), 4.07-4.12 (m, O—CH₂, 2H), 4.14 (s, —O—CH₂COOH, 2H), 4.47 (s, Ar—O—CH₂CONH, 2H), 6.87 (s, O—C₆H₄—O, 4H), 7.18 (brs, NH, 1H).

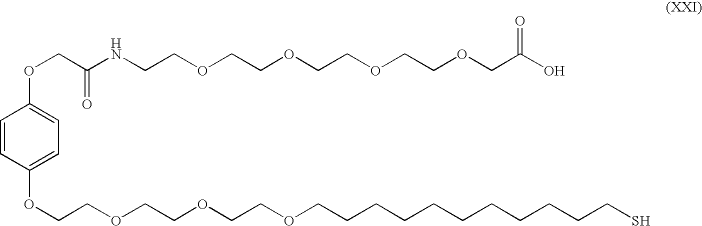

To a solution of compound (XX) (0.095 mmol, 72 mg) in dry tetrahydrofuran (4 mL), hydrazine monoacetate (0.39 mmol, 35 mg) in dry dimethylformamide (0.1 mL) was added. After 20 hours at room temperature, the solvent was evaporated under vacuum. The residue was dissolved in chloroform (50 mL), washed with water (3×20 mL), dried over anhydrous MgSO₄ and the solvent evaporated under vacuum. Finally, the product was purified by silica gel column chromatography (dichloromethane/methanol 20:1; then 10:1) to afford 55 mg (80% yield) of 1-(4-(2-(2-(2-(11-mercapto-undecyloxy)ethoxy)ethoxy)ethoxy)phenoxy)-2-oxo-6,9,12,15-tetraoxa-3-azahepta-decan-17-oic acid (XXI). ¹H NMR (200 MHz, CDCl₃): δ 1.15-1.45 (m, CH₂, 14H), 1.50-1.70 (m, CH₂, 4H), 2.52 (q, J=7.3 Hz, HS—CH₂—, 2H), 3.45 (t, J=6.6 Hz, O—CH₂—2H) 3.50-3.80 (m, —CH₂—O, CONH—CH₂—, 24H), 3.80-3.90 (m, O—CH₂—, 2H), 4.04-4.14 (m, O—CH₂—, 2H), 4.13 (s, O—CH₂COOH, 2H), 4.46 (s, Ar—O—CH₂CONH, 2H), 6.86 (s, O—C₆H₄—O, 4H), 7.14 (brs CONH, 1H).

### Example 14

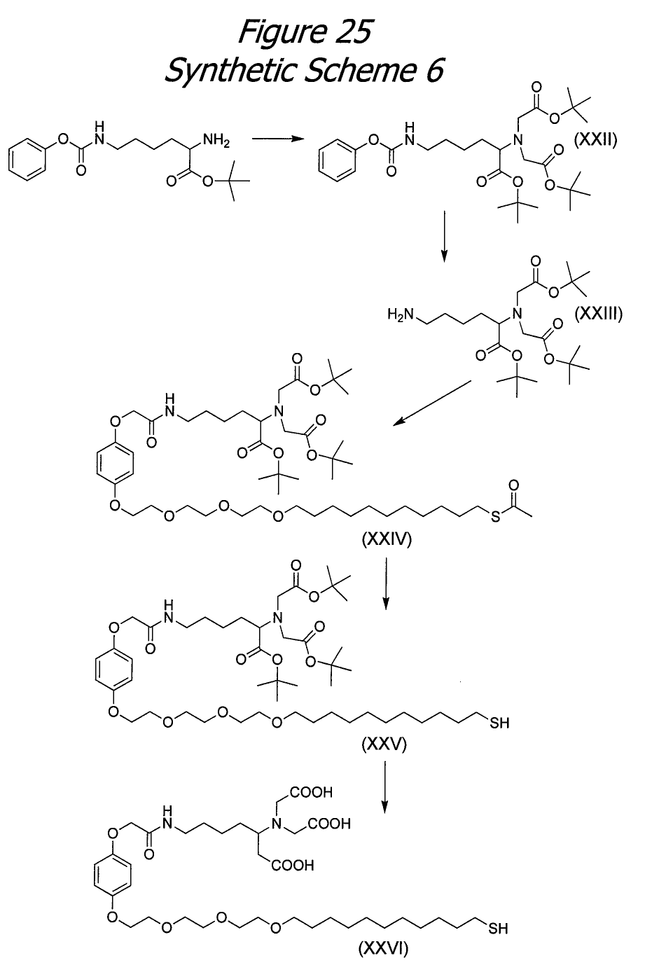

An alkanethiol of the present invention having a pendant metal ion chelating moiety useful for metal ion affinity chromatography, 2,2′-(1-carboxy-6-(2-(4-(2-(2-(2-(11-mercaptoundecyloxy)ethoxy)ethoxy)ethoxy)phenoxy)acetamido)hexan-2-ylazanediyl)-diacetic acid (XXVI), was prepared in three steps as outlined in FIG. 25 (Synthetic Scheme 6).

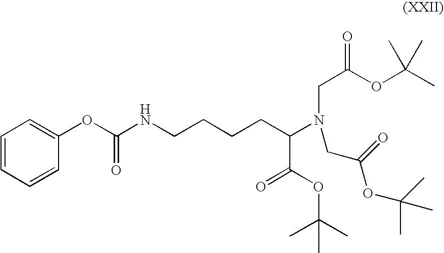

A suspension of Z-L-lysine t-butyl ester hydrochloride (13.41 mmol, 5.0 g) tert-butylbromoacetate (134 mmol, 26.15 g) and anhydrous potassium carbonate (67 mmol, 9.26 g) in dry acetonitrile (150 mL) was stirred under reflux for 24 hours. The solvent was evaporated under vacuum and the residue purified by silica gel column chromatography (petroleum ether; petroleum ether/chloroform 1:1; and chloroform) to afford 6.30 g (78% yield) of tert-Butyl 2,2′-(1-tert-butoxy-1-oxo-6-(phenoxycarbonyl-amino)hexan-2-ylazane-diyl)diacetate (XXII). TLC: (ethyl acetate/benzene 1:6) R_{f}=0.35.

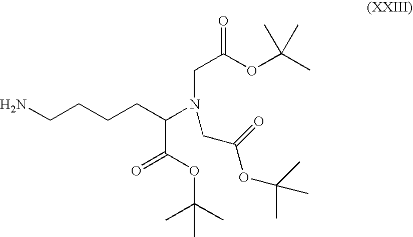

To a solution of compound (XXII) (4.99 mmol, 3.00 g) in ethanol (40 mL), 3 ml water, 1 ml acetic acid and 300 mg of 10% (w/w) Pd/C was added and hydrogen gas was bubbled into the solution for 18 hours with continuous stirring. The catalyst was removed by filtration and the filtrate evaporated under vacuum. The residue was dissolved in chloroform and washed twice with sodium hydroxide (1 N). Finally, the organic phase dried over anhydrous MgSO₄ and the solvent evaporated under vacuum to afford 1.95 g (91% yield) of tert-butyl 2,2′-(6-amino-1-tert-butoxy-1-oxohexan-2-ylazanediyl)-di-acetate (XXIII). TLC: (chloroform/methanol 10:1) R_{f}=0.15.

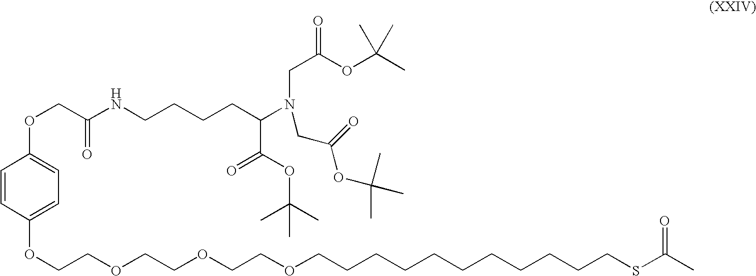

To a solution cooled to 0° C. containing compound of (XXIII) (1.32 mmol, 0.70 g) and N-hydroxysuccinimide (1.99 mmol, 230 mg) in dry tetrahydrofuran (20 mL), 1.99 mmol of dicyclohexylcarbodiimide (1.99 mmol, 410 mg) was added and the suspension stirred for 2 hours at 0° C. Then precipitate was removed by filtration and the supernatant added to a solution of compound of (XXIII) (1.62 mmol, 698 mg) in dry tetrahydrofuran (5 mL). Triethylamine (1.33 mmol, 0.19 mL) of was added and the reaction was stirred for 20 hours at room temperature. The solvent was evaporated under vacuum and the product purified by silica gel column chromatography (acetone/hexane 1:6) to afford 1.10 g (87% yield) of tert-Butyl 2,2′-(1-tert-butoxy-1-oxo-6-(2-(4-(22-oxo-3,6,9-trioxa-21-thiatricosyloxy)phenoxy)acetamido)hexan-2-ylazanediyl)-diacetate (XXIV). TLC: (ethyl ether) R_{f}=0.26. ¹H NMR (200 MHz, CDCl₃): δ 1.20-1.40 (m, CH₂, 16H), 1.45 (s, t-Bu, 18H), 1.46 (s, tBu, 9H), 1.50-1.80 (m, CH₂, 8H), 2.33 (s, CH₃COS, 3H), 2.86 (t, J=7.3 Hz, Ac—S—CH₂—, 2H), 3.25-3.78 (m, O—CH₂—CH₂—O, N—CH, N—CH₂, 17H), 3.80-3.90 (m, O—CH₂, 2H), 4.04-4.14 (m, O—CH₂, 2H), 4.42 (s, Ar—O—CH₂CON, 2H), 6.70 (brs, CONH, 1H), 6.86 (s, O—C₆H₄—O, 4H).

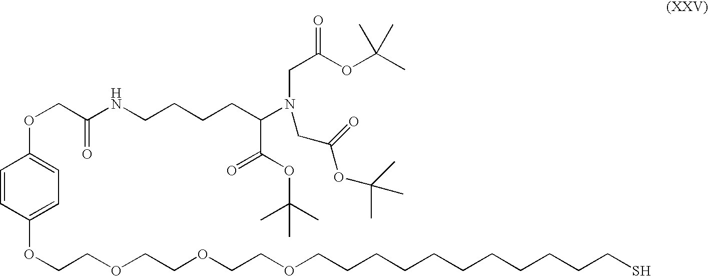

A solution of compound (XXIV) (0.722 mmol, 680 mg) and hydrazine monoacetate (7.22 mmol, 665 mg) in methanol (20 mL) was stirred continuously for 20 hours. Then solvent was evaporated under vacuum and the residue dissolved in chloroform, washed with water, dried over anhydrous MgSO₄ and evaporated under vacuum. Finally, the product was purified by silica gel column chromatography (acetone/hexane 1:6) to yield 500 mg (77% yield) of tert-butyl 2,2′-(1-tert-butoxy-1-oxo-6-(2-(4-(22-oxo-3,6,9-trioxa-21-thiatricosyloxy)phenoxy)acetamido)hexan-2-ylazanediyl)diacetate (XXV). TLC: (ethyl ether) R_{f}=0.25. ¹H NMR (200 MHz, CDCl₃): δ 1.20-1.40 (m, CH₂, 16H), 1.46 (s, tBu, 27H), 1.50-1.70 (m, CH₂, 8H), 2.52 (t, J=7.3 Hz, S—CH₂—, 2H), 3.25-3.78 (m, O—CH₂—CH₂—O, N—CH, N—CH₂, 17H), 3.80-3.90 (m, O—CH₂, 2H), 4.04-4.14 (m, O—CH₂, 2H), 4.42 (s, Ar—O—CH₂CON, 2H), 6.78 (brs, CONH, 1H), 6.86 (s, O—C₆H₄—O, 4H).

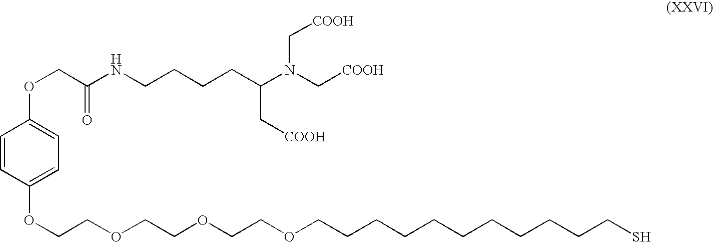

A solution cooled to 0° C. containing compound (XXV) (0.389 mmol, 350 mg) in chloroform (1 mL) and trifluoroacetic acid (5 mL) was stirred for 30 min at 0° C. and then for 4 hours at room temperature. The solvent was evaporated of under vacuum and the residue dissolved in dry toluene (20 mL). The toluene evaporated under vacuum and the product purified by silica gel column chromatography (chloroform/methanol/water 30:10:1) to afford 210 mg (64% yield) of 2,2′-(1-carboxy-6-(2-(4-(2-(2-(2-(11-mercaptoundecyloxy)ethoxy)ethoxy)ethoxy)phenoxy)acetamido)hexan-2-ylazane-diyl)diacetic acid (XXVI). as the trifluoroacetate salt. TLC (dichloromethane/methanol/water 30:10:1) R_{f}=0.20. ¹H NMR (200 MHz, CD₃OD): δ 1.20-1.40 (m, CH₂, 16H), 1.50-1.70 (m, CH₂, 8H), 2.48 (t, J=7.3 Hz, S—CH₂—, 2H), 3.40-3.78 (m, O—CH₂—CH₂—O, N—CH, N—CH₂, 17H), 3.80-3.90 (m, O—CH₂, 2H), 4.04-4.14 (m, O—CH₂, 2H), 4.43 (s, Ar—O—CH₂CON, 2H), 6.90 (s, O—C₆H₄—O, 4H).

### Example 15

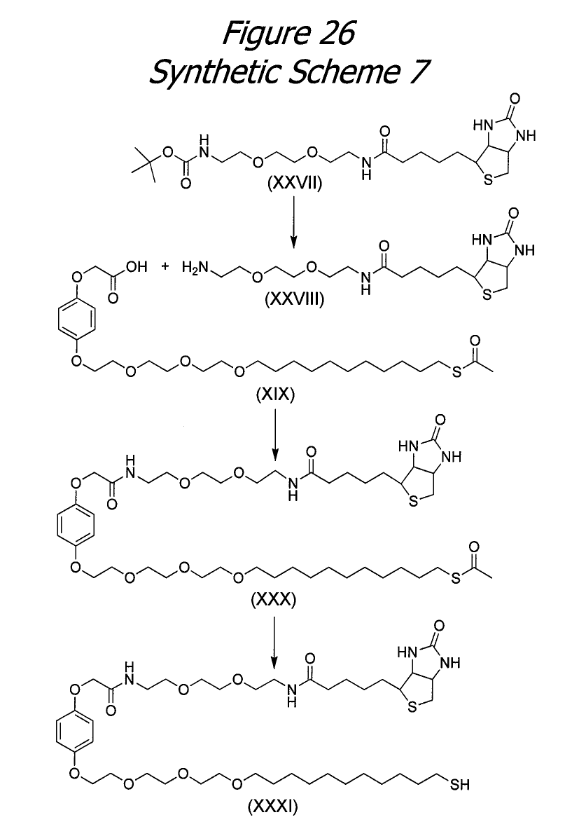

An alkanethiol of the present invention having a pendant biotin moiety, N-(2-(2-(2-(2-(4-(2-(2-(2-(11-mercaptoundecyloxy)ethoxy)ethoxy)ethoxy)phenoxy)-acetamido)ethoxy)ethoxy)-ethyl)-5-(2-oxohexahydro-1H-thieno[3,4-d]imidazol-4-yl)-pentanamide (XXXI), was prepared in three steps as outlined in FIG. 26 (Synthetic Scheme 7).

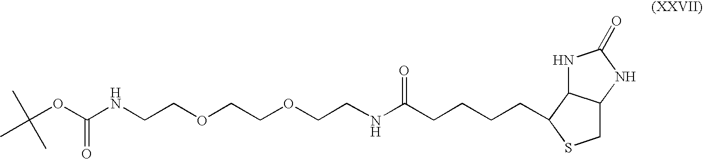

To a stirred solution of D-Biotin p-nitrophenylester (2.74 mmol, 1.00 g) in dimethyl-formamide/tetrahydrofuran (1:1, 10 mL), tert-butyl 2-(2-(2-aminoethoxy)ethoxy)ethyl-carbamate (3.28 mmol, 820 mg) was added. After continuous stirring for 18 hours the solvent was evaporated under vacuum. Finally, the product was purified by silica gel column chromatography (chloroform/methanol, 25:1 and 10:1) to afford 1.20 g (92% yield) of tert-butyl 2-(2-(2-(5-(2-oxohexahydro-1H-thieno[3,4-d]imidazol-4-yl)-pentanamido)ethoxy)ethoxy)ethylcarbamate (XXVII). TLC: (chloroform/methanol 10:1) R_{f}=0.20. ¹H NMR (500 MHz, CDCl₃): δ 1.46 (s, t-Bu, 9H), 1.40-1.50 (m, CH₂, 2H), 1.60-1.90 (m, CH₂, 4H), 2.36 (t, J=6.8 Hz, COCH₂, 2H), 2.82-2.88 (m, 1H, Biotin), 2.92-2.98 (m, 1H, Biotin), 3.15-3.25 (m, 1H, Biotin), 3.30-3.38 (m, N—CH₂—, 2H), 3.45-3.52 (m, N—CH₂—, 2H), 3.55-3.70 (m, O—CH₂, NH 10H), 4.40-4.48 (m, 1H, Biotin), 4.60-4.68 (m, 1H, Biotin), 6.30 (brs, CONH, 2H).

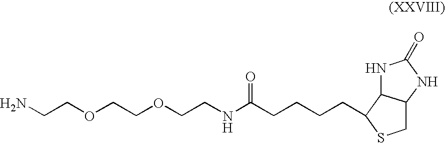

To a solution of compound (XXVII) (1.08 mmol, 0.51 g) in dichloromethane (1 mL) cooled to 0° C. was added trifluoroacetic acid (6 mL) and the resulting solution was stirred for 2 hours at room temperature. Then solvent was evaporated under vacuum and the residue dissolved in 10 mL of toluene. The toluene was removed under vacuum and the residue again dissolved in 10 mL of toluene. Finally, the toluene was removed under vacuum and the crude product N-(2-(2-(2-Aminoethoxy)ethoxy)ethyl)-5-(2-oxohex-ahydro-1H-thieno[3,4-d]imidazol-4-yl)pentanamide (XXVIII) (purity checked by ¹H NMR) was utilized for the next step without further purification.

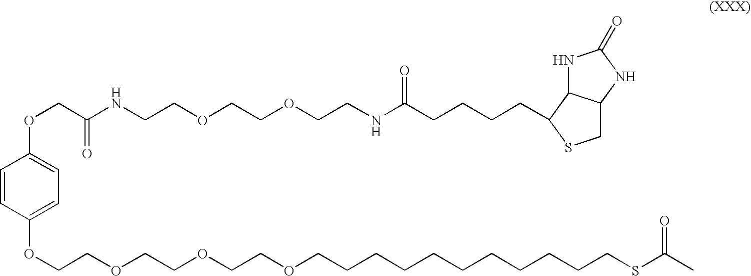

To a solution cooled to 0° C. containing compound (XIX) (1.00 mmol, 529 mg) and N-hydroxysuccinimide (1.50 mmol, 174 mg) in tetrahydrofuran (10 mL), dicyclohexylcarbodiimide (1.50 mmol, 309 mg) was added and the suspension stirred for 2 hours at 0° C. The precipitate was removed by filtration and the supernatant added to a solution of compound (XXVIII) (1.075 mmol) in tetrahydrofuran (5 mL). Triethylamine (1.075 mmol) was added and the suspension stirred continuously for 20 hours at room temperature. The solvent was evaporated under vacuum and the product purified by silica gel column chromatography (chloroform/methanol 25:1). The solvent was evaporated and the product again purified by silica gel column chromatography (ethyl acetate/methanol 10:1) to afford 250 mg (28% yield) of S-11-(2-(2-(2-(4-(2,13-dioxo-17-(2-oxohexahydro-1H-thieno[3,4-d]imidazol-4-yl)-6,9-dioxa-3,12-diazaheptadecyloxy)phenoxy)ethoxy)ethoxy)ethoxy)undecyl ethanethioate (XXX). TLC: (ethyl acetate/methanol 5:1) R_{f}=0.10. ¹H NMR (200 MHz, CDCl₃): δ 1.20-1.40 (m, CH₂, 14H), 1.40-1.50 (m, CH₂, 2H), 1.52-1.65 (m, CH₂, 4H), 1.65-1.80 (m, CH₂, 4H), 2.30 (t, J=6.8 Hz, COCH₂, 2H), 2.32 (s, CH₃COS, 3H), 2.86 (t, J=7.3 Hz, Ac—S—CH₂—, 2H), 2.81-2.98 (m, 2H, Biotin), 3.10-3.22 (m, 1H, Biotin), 3.46 (t, J=6.7 Hz, O—CH₂—, 4H), 3.50-3.78 (m, O—CH₂—CH₂—O, NH—CH₂—CH₂—O, 18H), 3.80-3.90 (m, O—CH₂, 2H), 4.02-4.13 (m, O—CH₂, 2H), 4.30-4.42 (m, 1H, Biotin), 4.47 (s, Ar—O—CH₂CON, 2H), 4.50-4.65 (m, 1H, Biotin), 5.8 (brs, CONH, 1H), 6.86 (s, O—C₆H₄—O, 4H), 7.10 (brs, CONH, 1H).

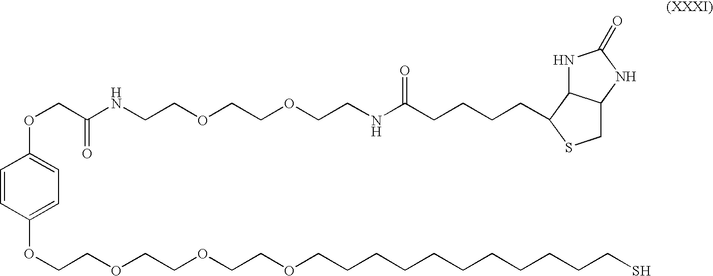

To a solution of compound (XXX) (0.50 mmol, 442 mg) in methanol (20 mL) was added hydrazine monoacetate (5.00 mmol) and the solution was stirred continuously for 20 hours at room temperature. The solvent was evaporated under vacuum and the product purified by silica gel column chromatography (ethyl acetate/methanol 10:1) to afford 290 mg (69% yield) of N-(2-(2-(2-(2-(4-(2-(2-(2-(11-mercaptoundecyloxy)-ethoxy)ethoxy)ethoxy)phenoxy)acetamido)ethoxy)ethoxy)ethyl)-5-(2-oxohexahydro-1H-thieno[3,4-d]-imidazol-4-yl)pentanamide (XXXI). TLC: (ethyl acetate/methanol 5:1) R_{f}=0.1. ¹H NMR (500 MHz, CDCl₃): δ 1.20-1.40 (m, CH₂, 14H), 1.40-1.50 (m, CH₂, 2H), 1.52-1.65 (m, CH₂, 4H), 1.65-1.80 (m, CH₂, 4H), 2.30 (t, J=6.8 Hz, COCH₂, 2H), 2.52 (q, J=7.3 Hz, S—CH₂—, 2H), 2.81-2.86 (m, 1H, Biotin), 2.90-2.98 (m, 1H, Biotin), 3.15-3.22 (m, 1H, Biotin), 3.46 (t, J=6.7 Hz, O—CH₂—, 4H), 3.50-3.78 (m, O—CH₂—CH₂—O, NH—CH₂—CH₂—O, 18H), 3.82-3.88 (m, O—CH₂, 2H), 4.05-4.13 (m, O—CH₂, 2H), 4.35-4.42 (m, 1H, Biotin), 4.47 (s, Ar—O—CH₂CON, 2H), 4.58-4.65 (m, 1H, Biotin), 6.82-6.85 (m, O—C₆H₄—O, 4H), 7.01 (brs, CONH, 1H), 7.11 (brs, CONH, 1H).

### Example 16

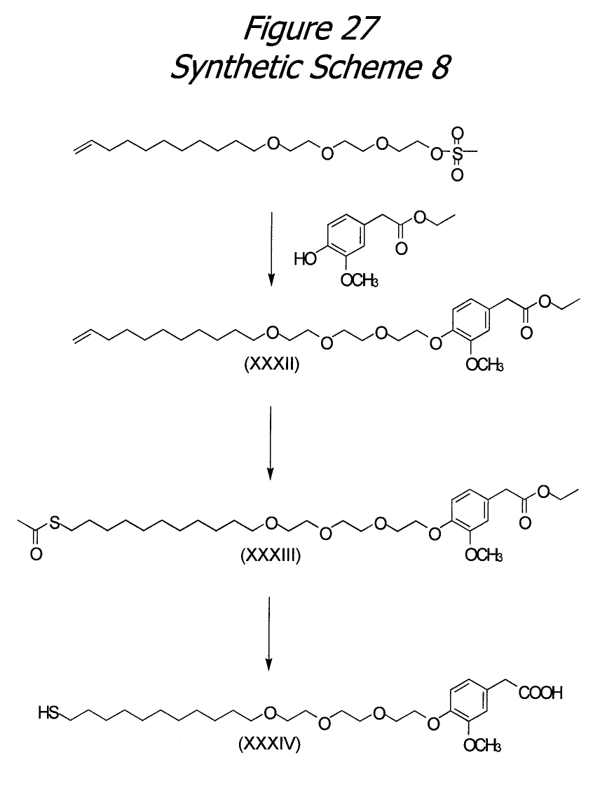

A further alkanethiol having a pendant reactive moiety useful as an intermediary for the preparation of self-assembled monolayers of the present invention, 3-methoxy-(2-(2-(2-(11-mercaptoundecyloxy)ethoxy)ethoxy)ethoxy)phenylacetic acid (XXXIV), was prepared in three steps as outlined in FIG. 27 (Synthetic Scheme 8).

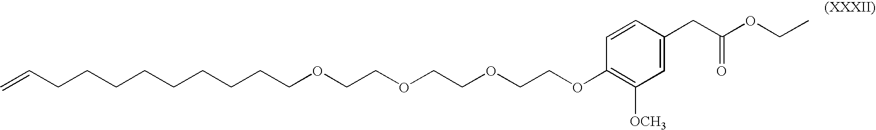

A mixture of methanesulfonic acid (26.5 mmol, 2.55 g), 2-{2-[2-(undec-10-enyloxy)ethoxy]ethoxy}ethyl ester (6.7 mmol), ethyl (4-hydroxy-3-methoxyphenyl)acetate (Aldrich #197971, 10.0 mmol, 2.10 g) and anhydrous potassium carbonate (20.0 mmol, 2.78 g) in dry acetonitrile (40 mL) was heated under reflux under an atmosphere of nitrogen for 20 hours. Then reaction mixture was cooled to room temperature, filtered, and concentrated under vacuum. The residue was purified by silica gel column chromatography (petroleum ether/acetone 6:1) to afford 1.87 g (64% yield) of ethyl 3-methoxy-4-(2-(2-(2-(undec-10-enyloxy)ethoxy)ethoxy)ethoxy)-phenylacetate (XXXII) as pale yellow oil.

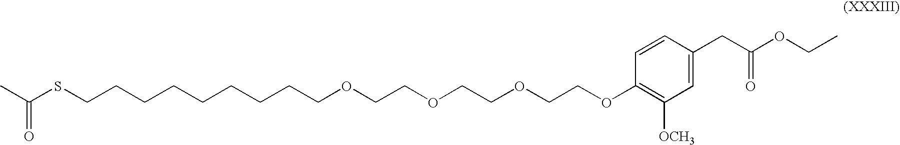

A solution of compound (XXXII) (5.41 mmol, 2.75 g), thioacetic acid (28.0 mmol) and AIBN (0.3 mmol, 51 mg) in methanol (75 mL) was irradiated in a photo-chemical reactor for 3 hours under an atmosphere of nitrogen. The solvent was evaporated under vacuum and the product purified by silica gel column chromatography (dichloromethane/diethyl ether 20:1) to afford 2.46 g (78% yield) of ethyl 3-methoxy-4-(22-oxo-3,6,9-trioxa-21-thiatricosyloxy)phenylacetate (XXXIII).

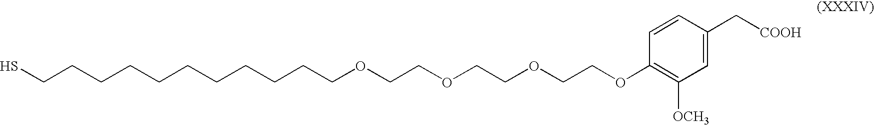

A solution of compound (XXXIII) (5.0 mmol), sodium hydroxide (5.5 mmol), methanol (30 mL) and water (15 mL) were stirred continuously at room temperature for 3 hours. The solution was acidified with dilute hydrochloric acid to pH 2 and the product extracted into chloroform (3×15 mL). The organic phases were combined, washed with water and dried over anhydrous magnesium sulfate. The solvent was evaporated under vacuum and the product purified by silica gel column chromatography (dichloromethane/methanol: 20:1; and 10:1) to afford 252 mg (72% yield) of 3-methoxy-4-(2-(2-(2-(11-mercaptoundecyloxy)ethoxy)ethoxy)ethoxy)phenylacetic acid (XXXIV).

While particular steps, elements, embodiments and applications of the present invention have been shown and described herein for purposes of illustration, it will be understood, of course, that the invention is not limited thereto since modifications may be made by persons skilled in the art, particularly in light of the foregoing teachings, without deviating from the spirit and scope of the invention. Accordingly, the invention is not limited except as by the appended claims.

What is claimed is:

1. A method for preparing an analyte-containing solution, the method comprising the steps of:
(a) providing an affinity capture surface comprising a substrate surface having adjacent first and second affinity capture zones, wherein:
(i) the first affinity capture zone comprises a first binary self-assembled monolayer comprising a plurality of affinity capture monomers and hydrophilic-terminated monomers associated with the substrate surface; and
(ii) the second affinity capture zone comprises a second binary self-assembled monolayer comprising a plurality of affinity capture monomers and hydrophobic-terminated monomers associated with the substrate surface, wherein the affinity capture monomers are capable of selectively retaining an analyte;
(b) contacting the affinity capture surface with the analyte to form analyte/affinity capture monomer complexes between the analyte and the affinity capture monomers; and
(c) cleaving the affinity capture monomers to release terminal portions of the affinity capture monomers and the analyte into a solution in contact with the affinity capture surface, thereby yielding the analyte-containing solution, generating a hydrophilic surface in the first affinity capture zone and generating a hydrophobic surface in the second affinity capture zone.

2. The method of claim 1 wherein the analyte-containing solution is an analyte-containing liquid droplet.

3. The method of claim 2, further comprising the step of evaporating the analyte-containing liquid droplet to transfer the evaporated analyte-containing liquid droplet to the hydrophilic first affinity capture zone.

4. The method of claim 3 wherein the first affinity capture zone is surrounded by the second affinity capture zone.

5. The method of claim 4 wherein the substrate surface of the affinity capture surface further has a non-wettable common surrounding zone surrounding the first and second affinity capture zones.

6. The method of claim 5 wherein the non-wettable common surrounding zone comprises a non-wettable self-assembled monolayer comprising a plurality of non-wettable monomers.

7. The method of claim 1 wherein the first binary self-assembled monolayer is comprised of at most 20% of the affinity capture monomers and at least 80% of the hydrophilic-terminated monomers, and the second binary self-assembled monolayer is comprised of at most 20% of the affinity capture monomers and at least 80% of the hydrophobic-terminated monomers.

8. The method of claim 7 wherein the first binary self-assembled monolayer is comprised of at most 10% of the affinity capture monomers and at least 90% of the hydrophilic-terminated monomers, and the second binary self-assembled monolayer is comprised of at most 10% of the affinity capture monomers and at least 90% of the hydrophobic-terminated monomers.

9. The method of claim 8 wherein the first binary self-assembled monolayer is comprised of 5% of the affinity capture monomers and 95% of the hydrophilic-terminated monomers, and the second binary self-assembled monolayer is comprised of 5% of the affinity capture monomers and at least 95% of the hydrophobic-terminated monomers.

10. The method of claim 1 wherein the affinity capture monomers have 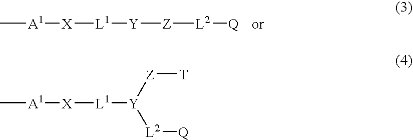
one of the following general formulas:
wherein:
A¹ is an anchoring moiety associated with the substrate surface;
X is alkylene;
L¹ is absent or is a protein adsorption resistant moiety;
Y is —O—Y¹, wherein Y¹ is aryl or substituted aryl, and wherein Y is bonded to L¹ and Z such that L¹ and Z are oriented in either an ortho or para relationship;
Z is a leaving group;
L² is absent or is a protein adsorption resistant moiety;
Q is an affinity capture moiety or reactive moiety; and
T is hydrogen, alkylene or aryl.

11. The method of claim 10 wherein the affinity capture monomers have the general formula:
-A¹-X-L¹-Y-Z-L²-Q (3)
wherein:
A¹ is —S— or —SCH₂CH₂CONH—;
X is C₃-C₁₈ alkylene;
L¹ is —(OCH₂CH₂)ₘ—, wherein m is an integer from 3 to 6;
Y is —O—Y¹, wherein Y¹ is aryl or substituted aryl, and wherein Y is bonded to L¹ and Z such that L¹ and Z are oriented in either an ortho or para relationship;
Z is —OCO—, —OCOO—, —OCONH—, —OSO₂—, —OPO₃—, —OCH₂COO—, —OCH₂CONH—, —OCH₂CH₂—, —OCH₂C₆H₄—, —OC₆H₄—, —OSi(CH₃)₂—, —OSi(CH₂CH₃)₂—, —OSi[CH(CH₃)₂]₂— or —OSi(C₆H₅)—;
L² is —(CH₂)ₙ— or —(CH₂CH₂O)ₙₙ—, wherein n an integer from 2 to 8 and nn is an integer from 2 to 6; and
Q is an affinity capture moiety or reactive moiety.

12. The method of claim 11 wherein:
A¹ is —S—;
X is —(CH₂)₁₁—;
L¹ is —(OCH₂CH₂)ₘ—, wherein m is 3 or 4;
Y is —O(C₆H₄)— and is bonded to L¹ and Z such that L¹ and Z are oriented in a para relationship;
Z is —OCH₂CONH—, —OCONH— or —OSi[CH(CH₃)₂]₂—;
L² is —(CH₂CH₂O)ₙₙ—, wherein nn is an integer from 2 to 6; and
Q is an affinity capture moiety or reactive moiety.

13. The method of claim 10 wherein:
the hydrophilic-terminated monomers have the general formula:
-A¹-X-L¹-T¹
wherein T¹ is a hydrophilic terminator; and
the hydrophobic-terminated monomers have the general formula:
-A¹-X-L¹-T²
wherein T² is a hydrophobic terminator.

14. The method of claim 13 wherein:
A¹ is —S— or —SCH₂CH₂CONH—;
X is C₃-C₁₈ alkylene;
L¹ is —(OCH₂CH₂)ₘ—, wherein m is an integer from 3 to 6;
T¹ is —OH, —OCH₂CONH₂, —OCONH₂ and —OCOCH₂OH;
T² is —OCH₃, —OCH₂CH₃, —OCOCH₃ and —OCOCF₃;
Y is —O—Y¹, wherein Y¹ is aryl or substituted aryl, and wherein Y is bonded to L¹ and Z such that L¹ and Z are oriented in either an ortho or para relationship;
Z is —OCO—, —OCOO—, —OCONH—, —OSO₂—, —OPO₃—, —OCH₂COO—, —OCH₂CONH—, —OCH₂CH₂—, —OCH₂C₆H₄—, —OC₆H₄—, —OSi(CH₃)₂—, —OSi(CH₂CH₃)₂—, —OSi[CH(CH₃)₂]₂— or —OSi(C₆H₅)₂—;
L² is —(CH₂)ₙ— or —(CH₂CH₂O)ₙₙ—, wherein n an integer from 2 to 8 and nn is an integer from 2 to 6; and
Q is an affinity capture moiety or reactive moiety.

15. The method of claim 1 wherein the affinity capture monomers are cleaved by electrochemical, chemical or photochemical means.

16. The method of claim 1 wherein the substrate surface comprises glass, metal, a polymeric material or silica.

17. The method of claim 15 wherein the substrate surface comprises gold or silver.

18. An affinity capture surface comprising a substrate surface having adjacent first and second affinity capture zones, wherein:
(a) the first affinity capture zone comprises a first binary self-assembled monolayer comprising a plurality of affinity capture monomers and hydrophilic-terminated monomers associated with the substrate surface; and
(b) the second affinity capture zone comprises a second binary self-assembled monolayer comprising a plurality of affinity capture monomers and hydrophobic-terminated monomers associated with the substrate surface,
and wherein the affinity capture monomers are capable of selectively retaining an analyte and are cleavable to release terminal portions of the affinity capture monomers and the analyte, thereby generating a hydrophilic surface in the first affinity capture zone and a hydrophobic surface in the second affinity capture zone.

19. The affinity capture surface of claim 18 wherein the first affinity capture zone is surrounded by the second affinity capture zone.

20. The affinity capture surface of claim 19 wherein the substrate surface of the affinity capture surface further has a non-wettable common surrounding zone surrounding the first and second affinity capture zones.

21. The affinity capture surface of claim 20 wherein the non-wettable common surrounding zone comprises a non-wettable self-assembled monolayer comprising a plurality of non-wettable monomers.

22. The affinity capture surface of claim 18 wherein the first binary self-assembled monolayer is comprised of at most 20% of the affinity capture monomers and at least 80% of the hydrophilic-terminated monomers, and the second binary self-assembled monolayer is comprised of at most 20% of the affinity capture monomers and at least 80% of the hydrophobic-terminated monomers.

23. The affinity capture surface of claim 22 wherein the first binary self-assembled monolayer is comprised of at most 10% of the affinity capture monomers and at least 90% of the hydrophilic-terminated monomers, and the second binary self-assembled monolayer is comprised of at most 10% of the affinity capture monomers and at least 90% of the hydrophobic-terminated monomers.

24. The affinity capture surface of claim 23 wherein the first binary self-assembled monolayer is comprised of 5% of the affinity capture monomers and 95% of the hydrophilic-terminated monomers, and the second binary self-assembled monolayer is comprised of 5% of the affinity capture monomers and at least 95% of the hydrophobic-terminated monomers.

25. The affinity capture surface of claim 18 wherein the affinity capture monomers have one of the following general formulas: 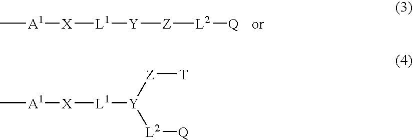
wherein:
A¹ is an anchoring moiety associated with the substrate surface;
X is alkylene;
L¹ is absent or is a protein adsorption resistant moiety;
Y is —O—Y¹, wherein Y¹ is aryl or substituted aryl, and wherein Y is bonded to L¹ and Z such that L¹ and Z are oriented in either an ortho or para relationship;
Z is a leaving group;
L² is absent or is a protein adsorption resistant moiety;
Q is an affinity capture moiety or reactive moiety; and
T is hydrogen, alkylene or aryl.

26. The affinity capture surface of claim 25 wherein the affinity capture monomers have the general formula:
-A¹-X-L¹-Y-Z-L²-Q (3)
wherein:
A¹ is —S— or —SCH₂CH₂CONH—;
X is C₃-C₁₈ alkylene;
L¹ is —(OCH₂CH₂)ₘ—, wherein m is an integer from 3 to 6;
Y is —O—Y¹, wherein Y¹ is aryl or substituted aryl, and wherein Y is bonded to L¹ and Z such that L¹ and Z are oriented in either an ortho or para relationship;
Z is —OCO—, —OCOO—, —OCONH—, —OSO₂—, —OPO₃—, —OCH₂COO—, —OCH₂CONH—, —OCH₂CH₂—, —OCH₂C₆H₄—, —OC₆H₄—, —OSi(CH₃)₂—, —OSi(CH₂CH₃)₂—, —OSi[CH(CH₃)₂]₂— or —OSi(C₆H₅)₂—;
L² is —(CH₂)ₙ— or —(CH₂CH₂O)ₙₙ—, wherein n an integer from 2 to 8 and nn is an integer from 2 to 6; and
Q is an affinity capture moiety or reactive moiety.

27. The affinity capture surface of claim 26, wherein:
A¹ is —S—;
X is —(CH₂)₁₁—;
L¹ is —(OCH₂CH₂)ₘ—, wherein m is 3 or 4;
Y is —O(C₆H₄)— and is bonded to L¹ and Z such that L¹ and Z are oriented in a para relationship;
Z is —OCH₂CONH—, —OCONH— or —OSi[CH(CH₃)₂]₂—;
L² is —(CH₂CH₂O)ₙₙ—, wherein nn is an integer from 2 to 6; and
Q is an affinity capture moiety or reactive moiety.

28. The affinity capture surface of claim 25 wherein:
the hydrophilic-terminated monomers have the general formula:
-A¹-X-L¹-T¹
wherein T¹ is a hydrophilic terminator; and
the hydrophobic-terminated monomers have the general formula:
-A¹-X-L¹-T²
wherein T² is a hydrophobic terminator.

29. The affinity capture surface of claim 28 wherein:
A¹ is —S— or —SCH₂CH₂CONH—;
X is C₃-C₁₈ alkylene;
L¹ is —(OCH₂CH₂)ₘ—, wherein m is an integer from 3 to 6;
T¹ is —OH, —OCH₂CONH₂, —OCONH₂ and —OCOCH₂OH;
T² is —OCH₃, —OCH₂CH₃, —OCOCH₃ and —OCOCF₃;
Y is —O—Y¹, wherein Y¹ is aryl or substituted aryl, and wherein Y is bonded to L¹ and Z such that L¹ and Z are oriented in either an ortho or para relationship;
Z is —OCO—, —OCOO—, —OCONH—, —OSO₂—, —OPO₃—, —OCH₂COO—, —OCH₂CONH—, —OCH₂CH₂—, —OCH₂C₆H₄—, —OC₆H₄—, —OSi(CH₃)₂—, —OSi(CH₂CH₃)₂—, —OSi[CH(CH₃)₂]₂— or —OSi(C₆H₅)₂—;
L² is —(CH₂)ₙ— or —(CH₂CH₂O)ₙₙ—, wherein n an integer from 2 to 8 and nn is an integer from 2 to 6; and
Q is an affinity capture moiety or reactive moiety.

30. The affinity capture surface of claim 18 wherein the substrate surface comprises glass, metal, a polymeric material or silica.

31. The affinity capture surface of claim 30 wherein the substrate surface comprises gold or silver.

32. A sample presentation device comprising the affinity capture surface of any one of claims 18-31.

33. A method of making the affinity capture surface of any one of claims 18-31 comprising the steps of:
(a) providing the substrate surface;
(b) associating the second binary self-assembled monolayer with the substrate surface;
(c) ablating the second binary self-assembled monolayer from the substrate surface in the first affinity capture zone; and
(d) associating the first binary self-assembled monolayer with the ablated surface in the first affinity capture zone.

34. A compound having one of the following general formulas: 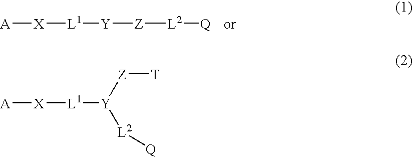
wherein:
A is an anchoring moiety;
X is alkylene;
L¹ is absent or is a protein adsorption resistant moiety;
Y is —O—Y¹, wherein Y¹ is aryl or substituted aryl, and wherein Y is bonded to L¹ and Z such that L¹ and Z are oriented in either an ortho or para relationship;
Z is a leaving group;
L² is absent or is a protein adsorption resistant moiety;
Q is an affinity capture moiety or reactive moiety; and
T is hydrogen, alkylene or aryl.

35. The compound of claim 34 wherein the compound has the general formula:
A-X-L¹-Y-Z-L²-Q (1)
wherein:
A is HS— or HSCH₂CH₂CONH—;
X is C₃-C₁₈ alkylene;
L¹ is —(OCH₂CH₂)ₘ—, wherein m is an integer from 3 to 6;
Y is —O—Y¹, wherein Y¹ is aryl or substituted aryl, and wherein Y is bonded to L¹ and Z such that L¹ and Z are oriented in either an ortho or para relationship;
Z is —OCO—, —OCOO—, —OCONH—, —OSO₂—, —OPO₃—, —OCH₂COO—, —OCH₂CONH—, —OCH₂CH₂—, —OCH₂C₆H₄—, —OC₆H₄—, —OSi(CH₃)₂—, —OSi(CH₂CH₃)₂—, —OSi[CH(CH₃)₂]₂— or —OSi(C₆H₅)₂—;
L² is —(CH₂)ₙ— or —(CH₂CH₂O)ₙₙ—, wherein n an integer from 2 to 8 and nn is an integer from 2 to 6; and
Q is an affinity capture moiety or reactive moiety.

36. The compound of claim 35 wherein:
A is HS—;
X is —(CH₂)₁₁—;
L¹ is —(OCH₂CH₂)ₘ—, wherein m is 3 or 4;
Y is —O(C₆H₄)— and is bonded to L¹ and Z such that L¹ and Z are oriented in a para relationship;
Z is —OCH₂CONH—, —OCONH— or —OSi[CH(CH₃)₂]₂—;
L² is —(CH₂CH₂O)ₙₙ—, wherein nn is an integer from 2 to 6; and
Q is an affinity capture moiety or reactive moiety.

37. The compound of claim 36, wherein the compound has the following structure:

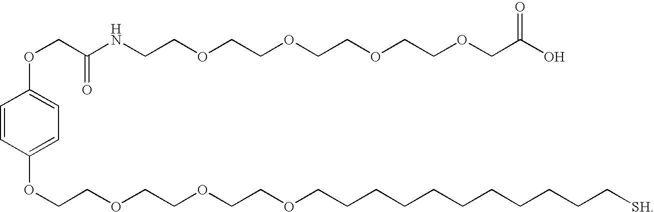

38. A self-assembled monolayer comprising:
a substrate surface; and
a plurality of affinity capture monomers immobilized on the substrate surface,
wherein the affinity capture monomers have one of the following general formulas: 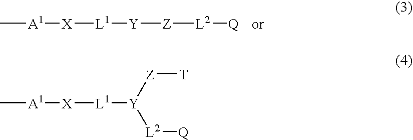
wherein:
A¹ is an anchoring moiety;
X is alkylene;
L¹ is absent or is a protein adsorption resistant moiety;
Y is —O—Y¹, wherein Y¹ is aryl or substituted aryl, and wherein Y is bonded to L¹ and Z such that L¹ and Z are oriented in either an ortho or para relationship;
Z is a leaving group;
L² is absent or is a protein adsorption resistant moiety;
Q is an affinity capture moiety or reactive moiety; and
T is hydrogen, alkylene or aryl.

39. The self-assembled monolayer of claim 38 wherein the affinity capture moieties have the following general formula:
-A¹-X-L¹-Y-Z-L²-Q (3)
wherein:
A¹ is —S— or —SCH₂CH₂CONH—;
X is C₃-C₁₈ alkylene;
L¹ is —(OCH₂CH₂)ₘ—, wherein m is an integer from 3 to 6;
Y is —O—Y¹, wherein Y¹ is aryl or substituted aryl, and wherein Y is bonded to L¹ and Z such that L¹ and Z are oriented in either an ortho or para relationship;
Z is —OCO—, —OCOO—, —OCONH—, —OSO₂—, —OPO₃—, —OCH₂COO—, —OCH₂CONH—, —OCH₂CH₂—, —OCH₂C₆H₄—, —OC₆H₄—, —OSi(CH₃)₂—, —OSi(CH₂CH₃)₂—, —OSi[CH(CH₃)₂]₂— or —OSi(C₆H₅)₂—;
L² is —(CH₂)ₙ— or —(CH₂CH₂O)ₙₙ—, wherein n an integer from 2 to 8 and nn is an integer from 2 to 6; and
Q is an affinity capture moiety or reactive moiety.

40. The self-assembled monolayer of claim 39 wherein:
A¹ is —S—;
X is —(CH₂)₁₁—;
L¹ is —(OCH₂CH₂)ₘ—, wherein m is 3 or 4;
Y is —O(C₆H₄)ₘ— and is bonded to L¹ and Z such that L¹ and Z are oriented in a para relationship;
Z is —OCH₂CONH—, —OCONH— or —OSi[CH(CH₃)₂]₂—;
L² is —(CH₂CH₂)ₙₙ—, wherein nn is an integer from 2 to 6; and
Q is an affinity capture moiety or reactive moiety.

41. The self-assembled monolayer of claim 38, further comprising:
a plurality of hydrophilic-terminated monomers, immobilized on the substrate surface, having the structure:
-A¹-X-L¹-T¹
wherein T¹ is a hydrophilic terminator; or
a plurality of hydrophobic-terminated monomers, immobilized on the substrate surface, having the structure:
-A¹-X-L¹-T²
wherein T² is a hydrophobic terminator.

42. The self-assembled monolayer of claim 41 wherein:
A¹ is —S— or —SCH₂CH₂CONH—;
X is C₃-C₁₈ alkylene;
L¹ is —(OCH₂CH₂)ₘ—, wherein m is an integer from 3 to 6;
T¹ is —OH, —OCH₂CONH₂, —OCONH₂ and —OCOCH₂OH;
T² is —OCH₃, —OCH₂CH₃, —OCOCH₃ and —OCOCF₃;
Y is —O—Y¹, wherein Y¹ is aryl or substituted aryl, and wherein Y is bonded to L¹ and Z such that L¹ and Z are oriented in either an ortho or para relationship;
Z is —OCO—, —OCOO—, —OCONH—, —OSO₂—, —OPO₃—, —OCH₂COO—, —OCH₂CONH—, —OCH₂CH₂—, —OCH₂C₆H₄—, —OC₆H₄—, —OSi(CH₃)₂—, —OSi(CH₂CH₃)₂—, —OSi[CH(CH₃)₂]₂— or —OSi(C₆H₅)₂—;
L² is —(CH₂)ₙ— or —(CH₂CH₂O)ₙₙ—, wherein n an integer from 2 to 8 and nn is an integer from 2 to 6; and
Q is an affinity capture moiety or reactive moiety.

43. The self-assembled monolayer of claim 38 wherein the substrate surface comprises glass, metal, a polymeric material or silica.

44. The self-assembled monolayer of claim 43 wherein the substrate surface comprises gold or silver.
